(19) United States
(12) Patent Application Publication
(10) Pub. No.: US 2025/0029254 A1
(43) Pub. Date: Jan. 23, 2025

(54) SYSTEMS AND METHODS FOR ANALYSIS OF COMPUTED TOMOGRAPHY (CT) IMAGES

(71) Applicant: ANNALISE-AI PTY LTD, Sydney, New South Wales (AU)

(72) Inventors: Dang-Dinh-Ang TRAN, Sydney, New South Wales (AU); Jarrel SEAH, Sydney, New South Wales (AU); Benjamin HACHEY, Sydney, New South Wales (AU); Xavier HOLT, Sydney, New South Wales (AU); Cyril TANG, Sydney, New South Wales (AU); Andrew JOHNSON, Sydney, New South Wales (AU); Marc NOTHROP, Sydney, New South Wales (AU); Kottal SAMARASINGHE, Sydney, New South Wales (AU); Jeffrey WARDMAN, Sydney, New South Wales (AU)

(21) Appl. No.: 18/716,097

(22) PCT Filed: Nov. 30, 2022
(86) PCT No.: PCT/AU2022/051429

§ 371 Date: Jun. 3, 2024

(30) Foreign Application Priority Data
Dec. 3, 2021 (AU) 2021903930
Aug. 17, 2022 (AU) 2022902344

(51) Int. Cl. G06T 7/00; G06T 17/00; G06V 10/762; G06V 10/82; G16H 30/20

(52) U.S. Cl. CPC G06T 7/0014; G06T 17/00; G06V 10/7625; G06V 10/82; G16H 30/20; G06T 2207/10081; G06T 2207/30016; G06T 2210/41

(57) ABSTRACT

Systems and methods for detecting visual findings such as visual anomaly findings in computed tomography (CT) scans. The method includes: receiving a series of anatomical images obtained from a computed tomography (CT) scan of a head of a subject; generating, using the series of anatomical images by a preprocessing layer: a spatial 3D tensor which represents a 3D spatial model of the head of the subject; generating, using the spatial 3D tensor by a convolutional neural network (CNN) model: at least one 3D feature tensor; and classifying, using at least one of the 3D feature tensors by the CNN model: each of a plurality of possible visual anomaly findings as being present versus absent, the plurality of possible visual anomaly findings having a hierarchal relationship based on a hierarchical ontology tree.

### CROSS-REFERENCE TO RELATED APPLICATIONS

This application refers to Australian Provisional Patent Application No. 2021903930 entitled “SYSTEMS AND METHODS FOR AUTOMATED ANALYSIS OF MEDICAL IMAGES”, filed Dec. 3, 2021 by the same applicant, Annalise-AI Pty Ltd, all the contents of which are incorporated by reference in its entirety herein below.

This application also refers to Australian Provisional Patent Application No. 2022902344 entitled “SYSTEMS AND METHODS FOR AUTOMATED ANALYSIS OF COMPUTED TOMOGRAPHY (CT) IMAGES”, filed Aug. 17, 2022 by the same applicant, Annalise-AI Pty Ltd, all the contents of which are incorporated by reference in its entirety herein below.

### TECHNICAL FIELD

Example embodiments relate to systems and methods for analyzing medical images, such as computed tomography (CT) scans.

### BACKGROUND

Generally, the manual interpretation of medical images performed by trained experts (such as e.g. radiologists) is a challenging task, due to the large number of possible findings that may be found. There is an ongoing need for improved computational methods, systems, services, and devices to automatically analyze anatomical images.

Non-contrast computed tomography of the brain (NCCTB) scans are a common imaging modality for patients with suspected intracranial pathology. In the emergency department (ED), NCCTB or brain computed tomography (CTB) scanning enables rapid diagnosis and the provision of timely care to patients who might otherwise suffer substantial morbidity and mortality. CT scanners are widely available and image acquisition time is short. Approximately 80 million NCCTB scans were conducted in 2021 in the United States alone.

The benefits of CT include more effective medical management by: determining when surgeries are necessary, reducing the need for exploratory surgeries, improving cancer diagnosis and treatment, reducing the length of hospitalisations, guiding treatment of common conditions such as injury, disease and stroke, and improving patient placement into appropriate areas of care, such as intensive care units (ICUs). The main advantages of CT are: to rapidly acquire images, provide clear and specific information, and image a small portion or all the body during the same examination/single session.

Despite the utility and widespread use of NCCTB scans, however, diagnostic error and misinterpretation remain prevalent. Error patterns have been reported for infarct detection, extra-axial masses, and thrombosis. ED physician interpretation of NCCTB scans is a substantial source of error. Reader inexperience, fatigue and interruptions appear to increase the likelihood of errors. In view of these problems, attempts have been made to develop artificial intelligence (AI) systems to mitigate these risks and assist clinicians with NCCTB interpretation.

A challenge is that predictions generated by deep learning models can be difficult to interpret by a user (such as, e.g., a clinician). Such models produce a score, probability or combination of scores for each class that they are trained to distinguish, which are often meaningful within a particular context related to the sensitivity/specificity of the deep learning model in detecting the clinically relevant feature associated with the class. Therefore, the meaning of each prediction should be evaluated in its specific context. This is especially problematic where deep learning models are used to detect a plurality of clinically relevant features, as a different specific context would have to be presented and understood by the user for each of the plurality clinical relevant features.

Accordingly, there is also an ongoing need for automated analysis systems to communicate the statistical results of deep learning models more effectively to a user in a fast, simple and intuitive manner that imposes lower cognitive load on the user, thereby enabling the user to make an informed clinical decision.

Computational methods for providing automated analysis of anatomical images may be provided in the form of an online service, e.g. implemented in a cloud computing environment. This enables the computational resources required for analysis to be provided and managed in a flexible manner, and reduces the requirement for additional computing power to be made available on-premises (e.g. in hospitals and other clinical environments). This approach also enables analysis services to be made available in low-resource environments, such as developing countries. However, in the presence of bandwidth constraints (e.g. in developing countries and/or remote locations with poor Internet bandwidth, and particularly in cases where there is a high volume of images such as CT scans), returning processed data to the user in a timely manner may be challenging. This is particularly crucial in situations where the user must wait for data to be retrieved in real-time, e.g. when reviewing a study at an on-premises workstation. The user experiencing such delay or seeing flickering on the screen because the image is being retrieved as they are attempting to view it can represent a significant barrier to adoption of automated solutions for medical image analysis. Further, such issues can undermine the benefits of automated medical image analysis, as they can reduce the amount of expert time that is saved by performing some of the analysis in an automated fashion.

Therefore, there is also an ongoing need for improved methods for communicating the results of medical image analysis to a user in a manner that efficiently produces clinically useful outputs for clinical decision support.

### SUMMARY

An example embodiment includes a deep learning model in the form of a CNN model which includes or is trained using a hierarchical ontology tree to detect/classify a wide range of NCCTB clinician findings (classifications) from images of a CT scan, significantly improving radiologist diagnostic accuracy.

An example embodiment includes a method for generating a spatial 3D tensor from a series of images from a computed tomography (CT) scan of a head of a subject. The method includes classifying certain potential visual anomaly findings in the spatial 3D tensor. The method includes generating a respective segmentation mask for certain visual anomaly findings found to be present. The method includes generating a plurality of 3D segmentation maps for each segmentation mask. Through a user device, a user can select a desired viewing of at least one of the segmentation maps overlaid on a representative anatomical slice of the subject for at least one anatomical plane.

In an example, the CNN model can determine localization of certain visual anomaly findings classified as being present in the CT scan. The localization can include left-right laterality, including identifying which of the left or right side of the head has certain visual anomaly findings classified as being present.

A comprehensive deep learning model for images of a CT scan of at least some example embodiments advantageously addresses common medical mistakes made by clinicians.

Example embodiments are advantageously more robust and reliable than other empirical models, specifically, deep learning models, in detecting radiological findings in images of a CT scan. Deep learning models in accordance with example embodiments may therefore be more clinically effective than others.

The CNN model may be trained by evaluating the performance of a plurality of neural networks in detecting the plurality of visual findings and selecting one or more best performing neural networks.

The plurality of visual findings may include visual findings selected from the ontology tree in Table 1.

The CNN model can classify an indication of whether each of the plurality of visual findings in the ontology tree is present in one or more of the images of a CT scan of the subject, the plurality of visual findings including all terminal leaves and internal nodes of the hierarchical ontology tree. An internal node can have one or more terminal leaves stemming from the internal node. Another example nomenclature is parent nodes and child nodes (and optionally grandchild nodes, etc.), in which a parent node can have one or more child nodes stemming from the parent node. In an example, each internal node uniquely branches to one or more terminal leaves. Another example nomenclature is hierarchal levels, in which a positive classification (a visual finding being present) in a lower hierarchal level necessarily means that the higher hierarchal level should also have a positive classification (e.g., a visual finding of a more general nature also being present). In other words, the neural network may output a prediction for each of the plurality of visual findings, which include both internal nodes and terminal leaves in the hierarchical ontology tree.

Further example embodiments include methods of diagnosis and/or treatment of one or more medical conditions in a subject, such methods comprising analyzing an anatomical image from the subject, or a portion thereof, using a method according to any one or more example embodiments.

Further example embodiments, aspects, advantages, and features of example embodiments will be apparent to persons skilled in the relevant arts from the following description of various embodiments. It will be appreciated, however, that the example embodiments are not limited to the embodiments described, which are provided in order to illustrate the principles of the example embodiments as defined in the foregoing statements and in the appended claims, and to assist skilled persons in putting these principles into practical effect.

According to a further aspect, there is provided a method for detecting a plurality of visual findings in a series of anatomical images from a computed tomography (CT) scan of a head of a subject, the method comprising the steps of:
providing a series of anatomical images from a computed tomography (CT) scan of a head of a subject;
inputting the series of anatomical images into a convolutional neural network (CNN) component of a neural network to output a feature vector;
computing an indication of a plurality of visual findings being present in at least one of the series of anatomical images by a dense layer of the neural network that takes as input the feature vector and outputs an indication of whether each of the plurality of visual findings is present in at least one of the series of anatomical images, wherein the visual findings represent findings in the series of anatomical images;
wherein the neural network is trained on a training dataset including, for each of a plurality of subjects, series of anatomical images, and a plurality of labels associated with the series of anatomical images and each of the respective visual findings, wherein the plurality of visual findings is organised as a hierarchical ontology tree and the training comprises evaluating performance of the neural network at different levels of the hierarchy of the ontology tree.

Advantageously, embodiments of the invention may employ a deep learning model trained to detect/classify a wide range of NCCTB clinician findings, significantly improving radiologist diagnostic accuracy.

The training of the deep learning model may be in combination with a plurality of other radiological findings, e.g. 195 radiological findings for the head of a subject. For every image of a CT scan/study, in one example, the inventors generated labels for each of the 195 findings enabling them to prevent a deep learning model from learning incorrect data correlations, for instance, between highly correlated radiological findings detectable in the head of a subject.

A comprehensive deep learning model for images of a CT scan/study of a CT study/scan embodying the invention advantageously addresses common medical mistakes made by clinicians.

Embodiments of the present invention are advantageously more robust and reliable than other empirical models, specifically, deep learning models, in detecting radiological findings in images of a CT scan/study. Deep learning models embodying the invention may therefore be more clinically effective than others.

The neural network may be trained by evaluating the performance of a plurality of neural networks in detecting the plurality of visual findings and selecting one or more best performing neural networks.

The plurality of visual findings may include at least 80, at least 100 or at least 150 visual findings. The plurality of visual findings may include at least 80, at least 100 or at least 150 visual findings selected from Table 1.

The hierarchical ontology tree may include at least 50, at least 80, at least 100 or at least 150 terminal leaves. The neural network may output an indication of whether each of the plurality of visual findings is present in one or more of the images of a CT scan/study of the subject, the plurality of visual findings including all terminal leaves and internal nodes of the hierarchical ontology tree. In other words, the neural network may output a prediction for each of the plurality of visual findings, which include both internal nodes and terminal leaves in the hierarchical ontology tree.

The plurality of labels associated with at least a subset of the one or more images of a CT scan/study and each of the respective visual findings in the training dataset may be derived from the results of review of the one or more anatomical images by at least one expert. The plurality of labels for the subset of the images of a CT scan/study in the training dataset are advantageously derived from the results of review of the one or more images of a CT scan/study by at least two experts, preferably at least three or exactly three experts.

The plurality of labels for the subset of the images of a CT scan/study in the training dataset may be obtained by combining the results of review of the one or more anatomical images by a plurality of experts.

The plurality of labels associated with at least a subset of the one or more images of a CT scan/study and each of the respective visual findings in the training dataset may be derived from labelling using a plurality of labels organised as a hierarchical ontology tree. Preferably, at least one of the plurality of labels is associated with a terminal leaf in the hierarchical ontology tree, and at least one of the plurality of labels is associated with an internal node in the hierarchical ontology tree. As a result of the hierarchical structure, some of the plurality of labels will contain partially redundant information due to propagation of the label from a lower level to a higher (internal node) level. This may advantageously increase the accuracy of the prediction due to the model training benefitting both from high granularity of the findings in the training data as well as high confidence training data for findings at lower granularity levels.

In embodiments, the plurality of labels associated with the one or more images of a CT scan/study in the training dataset represent a probability of each of the respective visual findings being present in the at least one of the one or more images of a CT scan/study of a subject.

Labelling using a plurality of labels organised as a hierarchical ontology tree may be obtained through expert review as explained above. For example, a plurality of labels associated with at least a subset of the one or more images of a CT scan/study and each of the respective visual findings in the training dataset may be derived from the results of review of the one or more anatomical images by at least one expert using a labelling tool that allows the expert to select labels presented in a hierarchical object (such as e.g. a hierarchical menu). Using such tools, an expert may be able to select a visual finding as a terminal leaf of the hierarchical object, and the tool may propagate the selection through the hierarchy such that higher levels of the hierarchy (internal nodes) under which the selected label is located are also selected.

In embodiments, the indication of whether each of the plurality of visual findings is present in at least one of the one or more images of a CT scan/study represents a probability of the respective visual finding being present in at least one of the one or more images of a CT scan/study.

In embodiments, the plurality of labels associated with at least a further subset of the one or more images of a CT scan/study and each of the respective visual findings in the training dataset are derived from an indication of the plurality of visual findings being present in at least one of the one or more images of a CT scan/study obtained using a previously trained neural network.

In embodiments, the method further comprises computing a segmentation mask indicating a localisation in 3D space for at least one of the plurality of visual findings by a decoder that takes as input the feature vector and outputs an indication of where the visual finding is present in the one or more images of a CT scan/study. In embodiments, the decoder is the expansive path of a U-net where the contracting path is provided by the CNN component that outputs the feature vector.

In embodiments, the neural network is trained by evaluating the performance of a plurality of neural networks (the plurality of neural networks being trained from a labelled dataset generated via consensus of radiologists) in detecting the plurality of visual findings and in detecting the localisation in 3D space of any of the plurality of visual findings in 3D space that are predicted to be present.

In embodiments, the neural network takes as input a standardised 3D volume. CT brain studies consist of a range of 2D images, and the number of these images varies a lot depending on the slice thickness, for example. Before these images are passed for training the AI model they need to be standardised by converting them into a fixed shape and voxel spacing. Describing from a different perspective, there is provided a plurality of images of a CT scan/study (such as e.g. from about 100 to about 450) to the neural network as an input to the entire machine learning pipeline. The neural network produces as output an indication of a plurality of visual findings being present in any one of the plurality of images. In a preferred embodiment, the number of slices may be about 126 in order to standardise the volume. This quantity may be forced by a pre-processing step.

In embodiments, the neural network is trained by evaluating the performance of a plurality of neural networks in detecting the plurality of visual findings, wherein the performance evaluation process takes into account the correlation between one or more pairs of the plurality of visual findings.

In embodiments, the plurality of labels associated with the one or more anatomical images and each of the respective visual findings is generated via consensus of imaging specialists. The visual findings may be radiological findings in anatomical images comprising one or more images of a CT scan/study, and the imaging specialists may be radiologists. The displaying may be performed through a user-interface.

In embodiments, the method further comprises repeating the method for one or more further first values, each of which provide(s) an indication of whether a respective further visual finding is present in at least one of one or more anatomical images of a subject, wherein each further first value is an output generated by a deep learning model trained to detect at least the further visual finding in anatomical images.

Advantageously, improved usability may be further facilitated by enabling the user to interact with the results of the deep learning models in an efficient manner by performing one or more of: selectively displaying a particular prediction of set or predictions associated with a particular, user-selected, radiological finding, selectively displaying a subset of the radiological findings for which a prediction is available, displaying a subset of the radiological findings as priority findings separately from the remaining of the radiological findings.

Accordingly, in embodiments the method further comprises displaying a list of visual findings comprising at least the first visual finding on a user interface, wherein the step of displaying the transformed first value, and optionally the predetermined fixed threshold and transformed second value(s) is triggered by a user selecting the first visual finding. In embodiments, the user selecting the first visual finding comprises the user placing a cursor displayed on the user interface over the first visual finding in the displayed list.

Within the context of the present disclosure, displaying a list of visual findings comprises displaying a plurality of text strings, each representing a radiological finding associated with a respective visual finding.

In embodiments, the method further comprises displaying a list of visual findings comprising at least the first visual finding on a user interface, wherein the visual findings are organised as a hierarchical ontology tree and the step of displaying the list of visual findings comprises displaying the visual findings that are at a single level of the hierarchical ontology tree, and displaying the children of a user-selected displayed visual finding, optionally wherein the user selecting a displayed visual finding comprises the user placing a cursor displayed on the user interface over the displayed visual finding in the displayed list.

The list of visual findings may comprise at least 100 visual findings. The selective displayed of subsets of visual findings organised as a hierarchical ontology tree enables the user to navigate through the results of deep learning analysis of anatomical images in an efficient manner.

The method may further comprise displaying a list of visual findings comprising at least the first visual finding on a user interface, wherein the list of visual findings is separated between at least a first sublist and a second sublist, wherein the first sublist comprises one or more visual findings that are priority findings, or an indication that there are no priority findings.

Advantageously, the selective display of particular subsets of visual findings in a ‘priority findings’ sub-list enables the user to quickly identify the image features that should be reviewed, thereby making the deep learning-aided analysis of the images of a CT scan/study more efficient. The set of visual findings included in the first sublist may be defined by default. Alternatively, one or more visual findings to be included in the first sublist and/or the second sublist may be received from a user.

The method may further comprise displaying a list of visual findings comprising at least the first visual finding on a user interface, wherein the list of visual findings is separated between a sublist comprising one or more visual findings that were detected in the anatomical images, and a sublist comprising one or more visual findings that were not detected in the anatomical images. The sublist comprising one or more visual findings that were detected in the anatomical images is separated between a first sublist and a second sublist, wherein the first sublist comprises one or more visual findings that are priority findings, or an indication that there are no priority findings.

The method may further comprise displaying at least one of the one or more anatomical images of the subject on a user interface, preferably a screen, and displaying a segmentation map overlaid on the displayed anatomical image(s) of the subject, wherein the segmentation map indicates the areas of the anatomical image(s) where the first visual finding has been detected, wherein the step of displaying the segmentation map is triggered by a user selecting the first visual finding in a displayed list of visual findings. The user selecting the first visual finding may comprise the user placing a cursor displayed on the user interface over the first visual finding in the displayed list.

The first value, the second value(s), and/or the segmentation map may be produced using a method according to any one or more embodiments of the first aspect.

An automated analysis of anatomical images using deep learning models may be improved by enabling the user to review the results of such automated analysis and provide feedback/corrective information in relation to a radiological finding that may have been missed by the automated analysis process, and using this information to train one or more improved deep learning model(s).

Accordingly, the method may further comprise displaying at least one of the one or more anatomical images of the subject and receiving a user selection of one or more areas of the anatomical image(s) and/or a user-provided indication of a first visual finding.

A user-provided indication of a first visual finding may be received by the user selecting a first visual finding from a displayed list of visual findings, or by the user typing or otherwise entering a first visual finding. Preferably, the method comprises receiving both a user selection of one or more areas of the anatomical image(s) and a user-provided indication of a first visual finding associated with the user-selected one or more areas.

Preferably, the method further comprises recording the user selected one or more areas of the anatomical image(s) and/or the user provided indication of the first visual finding in a memory, associated with the one or more anatomical image(s).

The method may further comprise using the user-selected one or more areas of the anatomical image(s) and/or the user-provided indication of the first visual finding to train a deep learning model to detect the presence of at least the first visual finding in anatomical images and/or to train a deep learning model to detect areas showing at least the first visual finding in anatomical images. The deep learning model trained to detect areas showing at least the first visual finding in anatomical images may be different from the deep learning model that trained to detect the presence of at least the first visual finding in anatomical images.

Using the user-selected one or more areas of the anatomical image(s) and/or the user-provided indication of the first visual finding to train a deep learning model to detect the presence of at least the first visual finding in anatomical images may comprise at least partially re-training the deep learning model that was used to produce the first value.

Using the user-selected one or more areas of the anatomical image(s) and/or the user-provided indication of the first visual finding to train a deep learning model to detect the areas showing at least the first visual finding in anatomical may comprise at least partially re-training the deep learning model that was used to produce a segmentation map indicating the areas of the anatomical image(s) where the first visual finding has been detected.

According to a further aspect, there is provided a method comprising:
receiving, by a processor, the results of a step of analysing a series of anatomical images from a computed tomography (CT) scan of a head of a subject using one or more deep learning models trained to detect and localise in 3D space at least a first visual finding in anatomical images, wherein the results comprise a plurality of segmentation maps obtained with for at least one anatomical plane, wherein a segmentation map indicates the areas of a respective anatomical image where the first visual finding has been detected; and
communicating, by the processor, the results of the analysing step to a user by sending to a user device at least the plurality of segmentation maps and a representative image corresponding to the respective anatomical image, wherein the processor is configured to select an initial desired viewing combination of segmentation map, anatomical plane and a user interface for the user device, and wherein the initial desired viewing combination is configured to selectively display the segmentation map overlaid on the information in the representative image corresponding to the respective anatomical image.

The representative image may be a “thumbnail” image (i.e. a smaller version of the original image/slice) for example.

In one example, the selective displaying may be sequentially progressive in response to a scrolling action by the user using the scroll wheel of a computer mouse, or by dragging interactive vertical or horizontal graphical user interface slider components displayed via the viewer component.

The anatomical plane may be any one from the group consisting of: sagittal, coronal or transverse.

The segmentation map may be chosen from a plurality of segmentation images of a CT scan/study. In an example, the user interface is configured to represent a particular anatomical entity, such as soft tissue, bone, stroke, subdural or brain.

Advantageously, in this aspect the most relevant or important results of a deep learning analysis are sent and displayed to enable the user to quickly and reliably visually confirm a finding detected by the deep learning model(s). This dramatically reduces the amount of data that must be provided to the user in order to communicate the results of the deep learning analysis, leading to a more efficient diagnosis process for the user. Additionally or alternatively, this dramatically reduces the amount of time required by a user, for example, a radiologist, to visually confirm the medical prediction(s) generated by the deep learning model(s) because the initial desired viewing combination is predicted and/or provided in the first instance from the analysing step to the user that is likely to be ideal for visual confirmation of the at least one detected radiological finding.

Accordingly, the step of sending a segmentation map image file and the respective anatomical image file is advantageously performed automatically in the absence of a user requesting the display of the results of the step of detecting the first visual finding.

The processor compressing the segmentation map image file may comprise the processor applying a lossless compression algorithm. The processor compressing the segmentation map image file may comprise the processor rendering the segmentation map as a PNG file.

The step of receiving a segmentation map image file and the respective anatomical image file is advantageously performed automatically in the absence of a user requesting the display of the results of the step of detecting the first visual finding.

As the skilled person understands, where a plurality of visual findings are detected in a single respective anatomical image, resulting in a plurality of segmentation map image files, the respective anatomical image file may only be sent to/received by the user device once. In other words, the methods may comprise determining that a segmentation map image file is associated with a respective medical image file that has already been sent to/received by the user device, and sending the segmentation map image file but not the respective anatomical image file.

In embodiments of any aspect, the segmentation map image file comprises a non-transparent pixel corresponding to every location of the respective anatomical image where the first visual finding has been detected.

Such image files may be referred to as transparent background files. The transparent file may be a binary transparent file. In a binary transparent file, every pixel is either transparent or not transparent (typically opaque). In embodiments, the transparent file comprises more than two levels of transparency. For example, the transparent file may comprise a first level for transparent pixels, a second level for opaque pixels, and a third level for semi-transparent pixels.

The segmentation map image file may comprise non-transparent pixels with a first level of transparency corresponding to the outline of every area of the respective anatomical image where the first visual finding has been detected, and non-transparent pixels with a second level of transparency corresponding to locations of the respective anatomical image where the first visual finding has been detected that are within an outlined area.

The second level of transparency may be higher (i.e. more transparent) than the first level of transparency. For example, the first level of transparency may specify opaque pixels, and the second level of transparency may specify semi-transparent pixels.

The first segmentation map image file and the respective anatomical image file may have substantially the same size. Every pixel of the first segmentation map image file may correspond to a respective pixel of the respective anatomical image file.

The method may further comprise resizing, by the processor or the user device processor, the first segmentation map image file and/or the respective anatomical image file such that every pixel of the first segmentation map image file corresponds to a respective pixel of the respective anatomical image file.

The method may further comprise repeating the steps of receiving and communicating or displaying using the results of a step of analysing the one or more anatomical images of a subject using one or more deep learning models trained to detect at least a further visual finding in anatomical images, wherein the results comprise at least a further segmentation map indicating the areas of a respective anatomical image where the further visual finding has been detected.

Any of the features related to automatically sending/receiving the results of a step of analysing one or more anatomical images of a subject may be performed in combination with the features associated with the communication of the first segmentation map image file as a separate file from the respective anatomical image file, or in the absence of the latter (e.g. in combination with the sending of the segmentation map information as part of a file that also comprises the respective anatomical image information). As such, also described herein are methods comprising: receiving, by a processor, the results of a step of analysing one or more anatomical images of a subject using one or more deep learning models trained to detect at least a first and optionally one or more further visual finding in anatomical images, wherein the results comprise at least a first (respectively, further) segmentation map indicating the areas of a respective anatomical image where the first (respectively, further) visual finding (has been detected; and communicating, by the processor, the result of the analysing step to a user by: sending to a user device at least the first (respectively, further) segmentation map and the respective anatomical image in the absence of a user requesting the display of the results of the step of detecting the first (of further) visual finding.

Similarly, also described herein are methods comprising: receiving, by a processor of a user device, the results of a step of analysing one or more anatomical images of a subject using one or more deep learning models trained to detect at least a first (respectively, further) visual finding in anatomical images, wherein receiving the results comprise receiving at least the first (respectively, further) segmentation map and the respective anatomical image in the absence of a user requesting the display of the results of the step of detecting the first (of further) visual finding; and displaying the information in the first (respectively, further) segmentation map to the user upon receiving a request to display the results of the step of detecting the first (of further) visual finding.

The methods described herein may further comprise the step of determining an order of priority for a plurality of visual findings, wherein the step of sending/receiving a segmentation map image file is performed automatically for the plurality of visual findings according to the determined order of priority.

The method may further comprise the processor communicating and/or the user computing device processor displaying a list of visual findings comprising the plurality of visual findings, wherein determining an order of priority for the plurality of visual findings comprises receiving a user selection of a visual finding in the displayed list of visual findings and prioritising visual findings that are closer to the user selected visual finding on the displayed list, relative to the visual findings that are further from the user selected visual finding.

The segmentation map may be produced using a method according to any one or more embodiments of the first, second or third aspect, and/or a user interface providing for user selection of, and interaction with, visual findings may be provided using a method according to any one or more embodiments of the seventh aspect.

In further aspects, there are provided methods of diagnosis and/or treatment of one or more medical conditions in a subject, such methods comprising analysing an anatomical image from the subject, or a portion thereof, using a method according to any one or more embodiments of the first, second or third aspect.

Further aspects, advantages, and features of embodiments of the invention will be apparent to persons skilled in the relevant arts from the following description of various embodiments. It will be appreciated, however, that the invention is not limited to the embodiments described, which are provided in order to illustrate the principles of the invention as defined in the foregoing statements and in the appended claims, and to assist skilled persons in putting these principles into practical effect.

### BRIEF DESCRIPTION OF THE FIGURES

Reference will now be made, by way of example, to the accompanying drawings which show example embodiments, and in which:

FIG. 1 is a block diagram illustrating an exemplary system according to an example embodiment;

FIG. 2 is a schematic illustration of a CNN model implemented by the system of FIG. 1 according to an example embodiment;

FIG. 3A illustrates an example of the CNN model according to example embodiments;

FIGS. 3B to 3G illustrate an example of the CNN model according to example embodiments;

FIGS. 4A and 4B are schematic illustrations of model generation and deployment stages of the CNN model, respectively;

FIGS. 5A and 5B show examples of segmentation maps provided by the CNN model overlaid on medical images, including exemplary interactive user interface screens of a viewer component according to an example embodiment;

FIG. 6A to 6D show exemplary interactive user interface screens of a viewer component according to an example embodiment;

FIG. 7A is a block diagram of an exemplary microservices architecture of a medical image analysis system according to an example embodiment;

FIG. 7B is a signal flow diagram illustrating an exemplary method for initiating processing of medical imaging study results within the embodiment of FIG. 7A;

FIG. 7C is a signal flow diagram illustrating an exemplary method for processing and storage of medical imaging study results within the embodiment of FIG. 7A;

FIG. 8 is a signal flow diagram illustrating an exemplary method for providing image data to a viewer component within the embodiment of FIG. 7A;

FIG. 9 is a signal flow diagram illustrating a method of processing a segmentation image result within the embodiment of FIG. 7A;

FIG. 10 is a block diagram illustrating the exemplary system, according to an example embodiment;

FIGS. 11A to 11D show an exemplary workflow, from the time a study is being input into the system for the CNN model to perform predictions to and present the predictions to an output;

FIG. 12A shows an example of a medical image which is input into the system for the CNN model to perform predictions;

FIGS. 12B to 12F show different displays of the medical image of FIG. 12A by the system in 5 different windows (user interfaces or windowing types);

FIG. 13 illustrates an example of training the CNN model in accordance with an example embodiment;

FIG. 14 illustrates performance of the CNN model, radiologists unaided by the CNN model, and radiologists aided by the CNN model;

FIGS. 15A and 15B illustrate performance curves of the CNN model, radiologists unaided by the CNN model, and radiologists aided by the CNN model;

FIG. 16 illustrates performance of the CNN model on the recall and precision of radiologists;

FIGS. 17A to 17H illustrate performance of a study using the CNN model to detect acute cerebral infarction;

FIG. 18 illustrates 3D segmentation masks generated by the CNN model;

FIGS. 19A to 19K are Table 1; and

FIG. 20 is Table 2.

### DETAILED DESCRIPTION

This application refers to PCT patent application no. PCT/AU2021/050580 entitled “SYSTEMS AND METHODS FOR AUTOMATED ANALYSIS OF MEDICAL IMAGES”, filed Jun. 9, 2021 by the same applicant, Annalise-AI Pty Ltd, all the contents of which are incorporated by reference in its entirety.

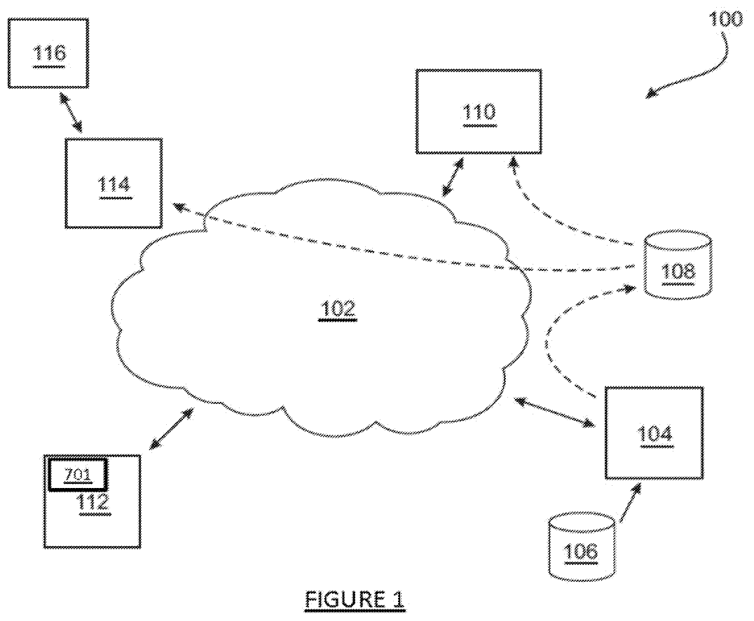

FIG. 1 is a block diagram illustrating an exemplary system 100 in which a network 102, e.g. the Internet, connects a number of components individually and/or collectively according to an example embodiment. The system 100 includes one or more processors. The system 100 is configured for training of machine learning models such as deep learning models and CNN models according to example embodiments, and for execution of the trained models to generate analysis of anatomical images. Analysis services provided by the system 100 may be served remotely, e.g. by software components executing on servers and/or cloud computing platforms that provide application programming interfaces (APIs) that are accessible via the network 102 (Internet). Additionally, or alternatively, the system 100 may enable on-site or on-premises execution of trained models for provision of local image analysis services and may be remotely accessible via a secure Virtual Private Network (VPN) connection. As will be apparent to skilled persons from the following description of example embodiments, systems having the general features of the exemplary system 100 may be implemented in a variety of ways, involving various hardware and software components that may be located on-site, at remote server locations, and/or provided by cloud computing services. It will be understood that all such variations available to persons skilled in the art, such as software engineers, fall within the scope of example embodiments. For simplicity, however, only a selection of exemplary embodiments will be described in detail.

The system 100 includes a model training platform 104, which comprises one or more physical computing devices, each of which may comprise one or more central processing units (CPUs), one or more graphics processing units (GPUs), memory, storage devices, and so forth, in known configurations. The model training platform 104 may comprise dedicated hardware, or may be implemented using cloud computing resources. The model training platform 104 is used in example embodiments, as described herein, to train one or more machine learning models to generate analysis of anatomical images. For the purposes of such training, the model training platform is configured to access a data store 106 that contains training data that has been specifically prepared, according to example embodiments, for the purposes of training the machine learning models. Trained models are stored within the system 100 within a data store 108, from which they may be made accessible to other components of the system 100. The data store 108 may comprise a dedicated data server, or may be provided by a cloud storage system.

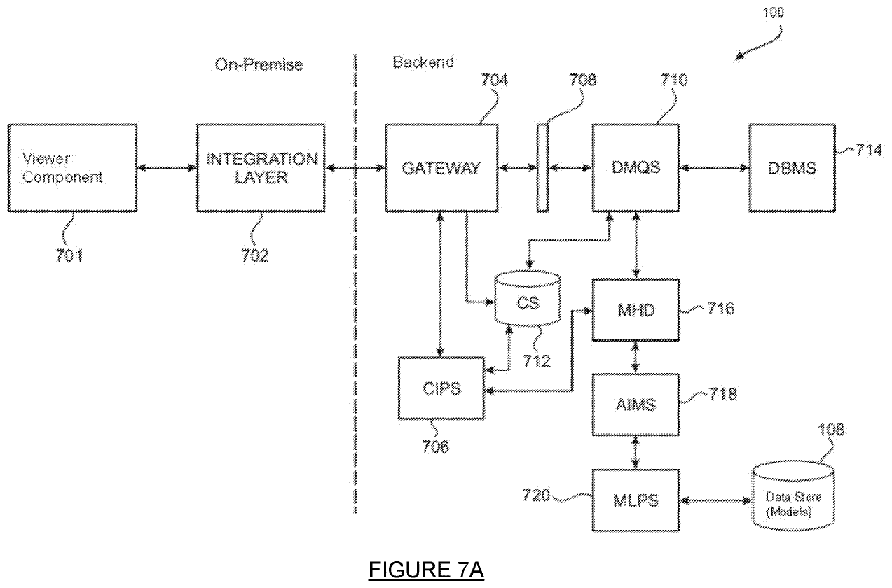

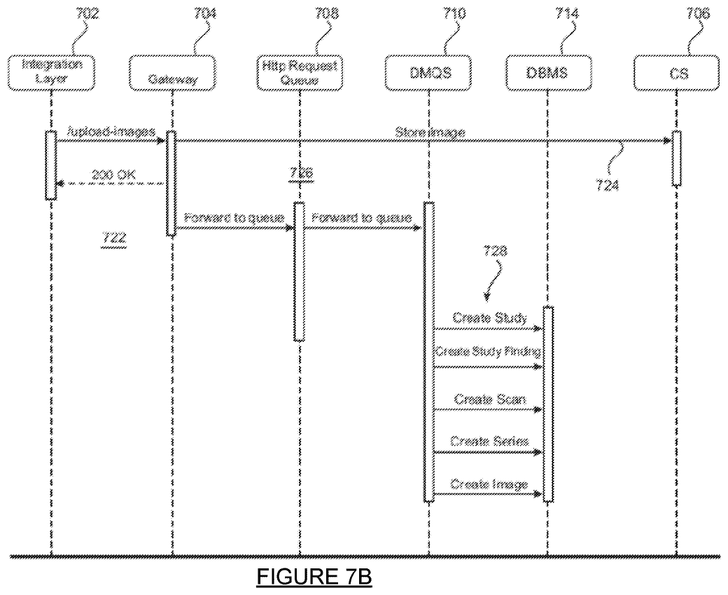

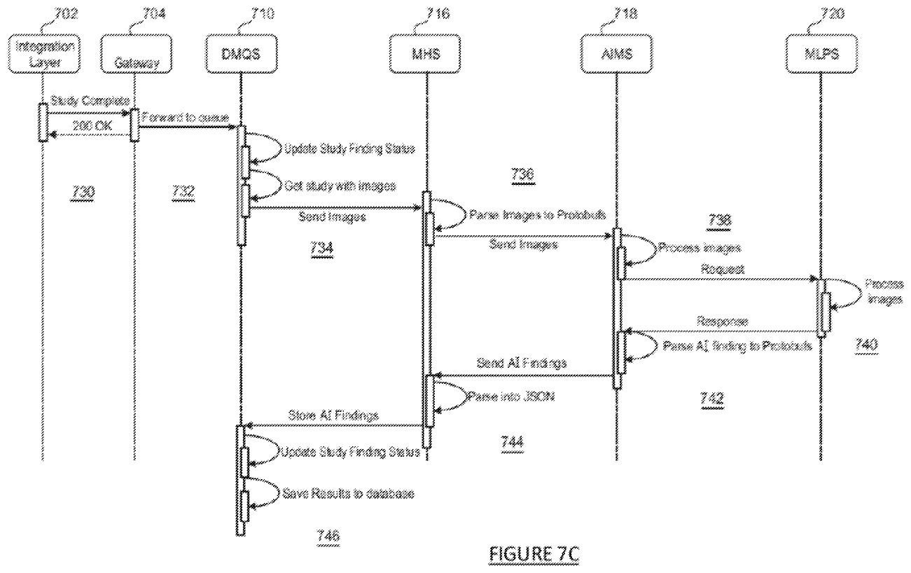

The system 100 further comprises a radiology image analysis server (RIAS) 110. It will be appreciated that a “radiology image” in this context may be any anatomical image, for example one or images of a CT scan or the brain and/or head. The anatomical images may be captured from different subjects. An exemplary RIAS 110, which is described in greater detail herein with reference to FIGS. 7A to 7C, is based on a microservices architecture, and comprises a number of modular software components developed and configured in accordance with described functions and processes of example embodiments. The RIAS 110 receives anatomical image data (e.g. images 204) that is transmitted from a source of anatomical image data, for example, where the anatomical image data captured and initially stored such as a clinic or its data centre. The transmission may occur in bulk batches of anatomical image data and prior to a user having to provide their decision/clinical report on a study. The transmission may be processed, controlled and managed by an integration layer 702 (FIG. 7A) (comprising integrator services of an integration adapter) installed at the clinic or a data centre, or residing at cloud infrastructure. In an example, the integration layer 702 is or includes a preprocessing layer which is configured to perform preprocessing of the images 204.

In the clinical use scenario, the RIAS 110 provides analysis services in relation to anatomical images captured by and/or accessible by user devices, such as medical (e.g. radiology) terminals/workstations 112, or other computing devices (e.g. personal computers, tablet computers, and/or other portable devices—not shown). The workstations 112 can include a viewer component 701 for displaying an interactive graphical interface (user interface or UI). The anatomical image data is analyzed by one or more software components of the RIAS 110, including through the execution of a CNN model 200. The RIAS 110 then makes the results of the analysis available and accessible to one or more user devices.

In other arrangements, which may exist in addition or as alternatives to the RIAS 110, an on-site radiology image analysis platform 114 may be provided. The on-site platform 114 comprises hardware, which may include one or more CPUs, and for example one or more GPUs, along with software that is configured to execute machine learning models in accordance with example embodiments. The on-site platform 114 may thereby be configured to provide anatomical image data analysis equivalent to that provided by a remote RIAS 110, accessible to a user of, e.g., a terminal 116. Machine learning models executed by the on-site platform 114 may be held in local storage and/or may be retrieved from the model data store 108. Updated models, when available, may be downloaded from the model data store 108, or may be provided for download from another secure server (not shown), or made available for installation from physical media, such as CD-ROM, DVD-ROM, a USB memory stick, portable hard disk drive (HDD), portable solid-state drive (SDD), or other storage media.

With regard to the preceding overview of the system 100, and other processing systems and devices described in this specification, terms such as ‘processor’, ‘computer’, and so forth, unless otherwise required by the context, should be understood as referring to a range of possible implementations of devices, apparatus and systems comprising a combination of hardware and software. This includes single-processor and multi-processor devices and apparatus, including portable devices, desktop computers, and various types of server systems, including cooperating hardware and software platforms that may be co-located or distributed. Physical processors may include general purpose CPUs, digital signal processors, GPUs, and/or other hardware devices suitable for efficient execution of required programs and algorithms.

Computing systems may include conventional personal computer architectures, or other general-purpose hardware platforms. Software may include open-source and/or commercially available operating system software in combination with various application and service programs. Alternatively, computing or processing platforms may comprise custom hardware and/or software architectures. As previously noted, computing and processing systems may comprise cloud computing platforms, enabling physical hardware resources, including processing and storage, to be allocated dynamically in response to service demands.

Terms such as ‘processing unit’, ‘component’, and ‘module’ are used in this specification to refer to any suitable combination of hardware and software configured to perform a particular defined task. Such a processing unit, components, or modules may comprise executable code executing at a single location on a single processing device, or may comprise cooperating executable code modules executing in multiple locations and/or on multiple processing devices. Where exemplary embodiments are described herein with reference to one such architecture (e.g. cooperating service components of the cloud computing architecture of the system 100 described with reference to FIGS. 7A to 7C) it will be appreciated that, where appropriate, equivalent functionality may be implemented in other embodiments using alternative architectures.

Software components embodying features in accordance with example embodiments may be developed using any suitable programming language, development environment, or combinations of languages and development environments, as will be familiar to persons skilled in the art of software engineering. For example, suitable software may be developed using the TypeScript programming language, the Rust programming language, the Go programming language, the Python programming language, the SQL query language, and/or other languages suitable for implementation of applications, including web-based applications, comprising statistical modeling, machine learning, data analysis, data storage and retrieval, and other algorithms. Implementation of example embodiments may be facilitated by the used of available libraries and frameworks, such as TensorFlow or PyTorch for the development, training and deployment of machine learning models using the Python programming language.

It will be appreciated by skilled persons that example embodiments involve the preparation of training data, as well as the implementation of software structures and code that are not well-understood, routine, or conventional in the art of anatomical image analysis, and that while pre-existing languages, frameworks, platforms, development environments, and code libraries may assist implementation, they require specific configuration and extensive augmentation (e.g. additional code development) in order to realize various benefits and advantages of example embodiments and implement the specific structures, processing, computations, and algorithms described herein with reference to the drawings.

The foregoing examples of languages, environments, and code libraries are not intended to be limiting, and it will be appreciated that any convenient languages, libraries, and development systems may be employed, in accordance with system requirements. The descriptions, block diagrams, flowcharts, tables, and so forth, presented in this specification are provided, by way of example, to enable those skilled in the arts of software engineering, statistical modeling, machine learning, and data analysis to understand and appreciate the features, nature, and scope of the example embodiments, and to put one or more example embodiments into effect by implementation of suitable software code using any suitable languages, frameworks, libraries and development systems in accordance with example embodiments without exercise of additional inventive ingenuity.

The program code embodied in any of the applications/modules described herein is capable of being individually or collectively distributed as a program product in a variety of different forms. In particular, the program code may be distributed using a computer readable storage medium having computer readable program instructions thereon for causing a processor to carry out features or aspects of the example embodiments.

Computer readable storage media may include volatile and non-volatile, and removable and non-removable, tangible media implemented in any method or technology for storage of information, such as computer-readable instructions, data structures, program modules, or other data. Computer readable storage media may further include random access memory (RAM), read-only memory (ROM), erasable programmable read-only memory (EPROM), electrically erasable programmable read-only memory (EEPROM), flash memory or other solid state memory technology, portable compact disc read-only memory (CD-ROM), or other optical storage, magnetic cassettes, magnetic tape, magnetic disk storage or other magnetic storage devices, or any other medium that can be used to store the desired information and which can be read by a computer. Computer readable program instructions may be downloaded via transitory signals to a computer, another type of programmable data processing apparatus, or another device from a computer readable storage medium or to an external computer or external storage device via a network.

Computer readable program instructions stored in a computer readable medium may be used to direct a computer, other types of programmable data processing apparatus, or other devices to function in a particular manner, such that the instructions stored in the computer readable medium produce an article of manufacture including instructions that implement the functions, acts, and/or operations specified in the flowcharts, sequence diagrams, and/or block diagrams. The computer program instructions may be provided to one or more processors of a general purpose computer, a special purpose computer, or other programmable data processing apparatus to produce a machine, such that the instructions, which execute via the one or more processors, cause a series of computations to be performed to implement the functions, acts, and/or operations specified in the flowcharts, sequence diagrams, and/or block diagrams.

Example embodiments can employ the Digital Imaging and Communications in Medicine (DICOM) standard, which is commonly used in medical imaging systems. The DICOM instance information model describes a hierarchical set of identifiers: the patient ID, and the study, series and service object pair (SOP) Unique Identifiers (UIDs). Each patient may have multiple studies. Each study may have multiple series. Each series may contain multiple SOPs. The four text identifiers in the DICOM standard have the following properties:
1. Patient ID—a non-globally unique identifier, intended to be unique within the context of an imaging service to identify individual patients'
2. Study UID—a globally unique ID (UID) capturing a set of image series, which are acquired within a single given context (e.g. a single visit);
3. Series UID—a globally unique ID of only one modality (e.g. x-ray) produced by only one piece of imaging equipment; and
4. SOP Instance UID—a globally unique ID referencing a single image (or non-image) DICOM instance.
   Regarding these identifiers:
a study may contain multiple series of different modalities;
a series may include multiple SOP instances (usually images); and
a DICOM instance may, for example, represent a single CT view, or a single frame of a stack of images in a computerized tomography (CT) series.

A series can comprise hundreds of images, whilst a study may comprise thousands of images. An image may have hundreds of KB, and a study may be up to several GB in size.

DICOM mechanisms ensure the uniqueness of each identifier that is required to be globally unique ID.

In example embodiments as described herein, medical images (also denoted “anatomical images”) produced by imaging equipment comprise image data, and image metadata including DICOM headers. Such images, also referred to simply as “DICOM images”, may be stored, transmitted between components of the system 100, employed for training of machine learning (ML) models, and provided as input for analysis by trained models.

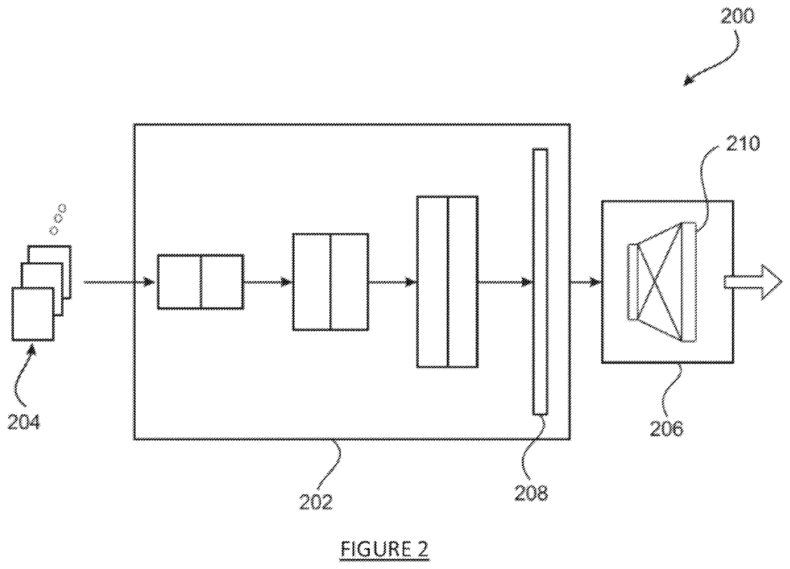

Referring to FIG. 2, example embodiments of a CNN model 200 (or simply “model”) are configured for analysis of anatomical images using statistical classifiers that include one or more deep learning models, and for communication of the results to user devices. The CNN model 200 can be executed by one or more processors of the system 100 (FIG. 1). In examples, the CNN model 200 comprises deep neural networks such as convolutional neural networks (ConvNets or CNNs). CNN components can be used as statistical classifiers/neural networks that take an image as input, and output a feature vector. An anatomical image (or medical image) is a two-dimensional image 204 of a body portion of a subject, obtained using anatomical imaging means such as e.g. a CT scanner. Exemplary body portions include: a head. For example, the body portion may be the head and the imaging modality may be non-contrast computed tomography of the brain (NCCTB) scanning, therefore the anatomical image may be a CT slice image 204 of a series of CT slice images 204 of the head.

The convolutional layers of a CNN take advantage of inherent properties of the medical images. The CNN takes advantage of local spatial coherence of medical images. This means that CNNs are generally able to dramatically reduce the number of operations needed to process a medical image by using convolutions on grids of adjacent pixels due to the importance of local connectivity. In addition to the CNNs, a transformer block is used and is connected to the encoder. Each map is then filled with the result of the convolution of a small patch of pixels, by applying a sliding window algorithm over the whole image. Each window includes a convolutional filter having weights and is convolved with the medical image (e.g. slide over the medical image spatially, computing dot products). The output of each convolutional filter is processed by a non-linear activation function, generating an activation map/feature map. The CNN has pooling layers which downscale the medical image. This is possible because features that are organized spatially are retained throughout the neural network, and thus downscaling them reduces the size of the medical image. When designing the CNN, the number of convolutional layers, filters and their size, alongside the type of activation function and the pooling method are carefully considered and selected to optimise model performance. Advantageously, transfer learning can be applied. Transfer learning includes using pre-trained weights developed by training the same model architecture on a larger (potentially unrelated) dataset, such as the ImageNet dataset (http://www.image-net.org). Training on the dataset related to the problem at hand by initialising with pre-trained weights allows for certain features to already be recognised and increases the likelihood of finding a global, or reduced local, minimum for the loss function than otherwise.

As used herein, references to using a deep neural network (DNN) to classify image data may in practice encompass using an ensemble of DNNs by combining the predictions of individual DNNs. Each ensemble may have the properties described herein. Similarly, references to training a DNN may in fact encompass the training of multiple DNNs as described herein, some or all of which may subsequently be used to classify image data, as the case may be.

In this example, the CNN model 200 comprises an ensemble of five CNN components trained using five-fold cross-validation. In an example, the CNN model comprises three heads (modules): one for classification, one for left-right localization in 3D space, and one for segmentation in 3D space. Models were based on the ResNet, Y-Net and Vision Transformer (ViT) architectures. An attention-per-token ViT head (vision transformer 394) is included to significantly improving the performance of classification of radiological findings for CTB studies (denoted visual anomaly findings or visual findings). Class imbalance is mitigated using class-balanced loss weighting and oversampling. Study endpoints addressed the performance of the classification performed by the CNN model. When segmentation output is present, the segmentation may be displayed to participants through the graphical user interface. The localization is a 3D tensor that will be sent to the viewer component 701 and overlaid on thumbnail images/slices of the CTB study. The 3D tensor may be a static size. A slice from a 3D tensor can be rendered by the viewer component 701 and scrolling through slices of the 3D tensors in the viewer component 701 is provided. The 3D tensor is reconstructed and all the required slices of the CTB study are stored in all required axes. The viewer component 701 provides a slice scrollbar graphical user interface component. The slice scrollbar may be oriented vertically, and comprises a rectangular outline. Coloured segments in the slice scrollbar, for example, purple colour, indicates that these slices have radiological findings predicted by the model. Large coloured segments visually indicate that there is a larger mass detected, whereas faint lines (thin segments) visually indicate that one or two consecutive slices with localization. The absence of coloured segments denotes a lack of radiological findings not detected by the model for these slices. The rest of the slices are loaded on-demand, when/if a radiologist scrolls through the other slices/regions. Grey coloured sections in the slice scrollbar indicates that slices that been pre-fetched. The ends of the slice scrollbar may have text labels appropriate for the anatomical plane being viewed. At one end of the slice scrollbar it indicates “cranial” while at the opposite distal end of the slice scrollbar it indicates “caudal” to provide a reference for the radiologist in the direction of scrolling. Other text labels may be “front” to “back”, or “right” to “left”.

FIG. 2 is a schematic illustration of the CNN model 200 according to an example embodiment. The feature vectors output by the CNN component 202 may be combined and fed into a dense layer 206, which is a fully connected layer that converts 3D feature tensors or 2D feature maps 208 into a flattened feature vector 210. The classification head is GlobalAveragePooling->batch norm->Dropout->Dense. In some embodiments, the feature vectors may be extracted following an average pooling layer. In some embodiments the dense layer is customised. In some embodiments, the dense layer is a final layer and comprises a predetermined number of visual findings as nodes. Each node then outputs an indication of the probability of the presence of each of a plurality of visual findings in at least one of the input images 204 of a CT scan. The input images may be preprocessed into a spatial 3D tensor which represents a 3D spatial model of the head of the subject. Alternatively or additionally, a prediction and optionally a confidence in this prediction may be output.

Deep learning models embodying the example embodiments can advantageously be trained to detect/classify a very high number of visual findings, such as, e.g., at least 195 potential visual anomaly findings as described in greater detail below with reference to Table 1. Such models may have been trained using NCCTB images (pixel data) where, in one example, labels were provided for each of the potential visual anomaly findings, enabling the CNN model 200 to be trained to detect combinations of findings, while preventing the CNN models 200 from learning incorrect correlations.

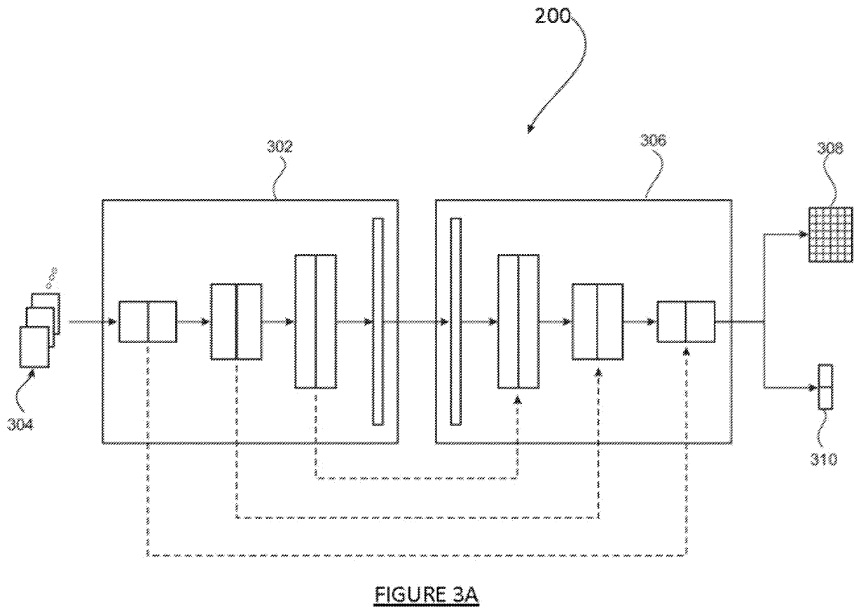

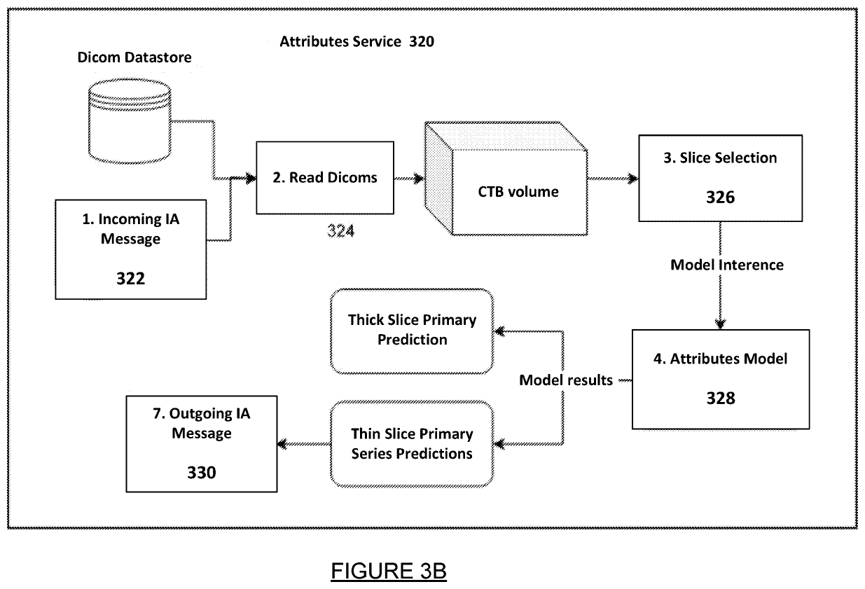

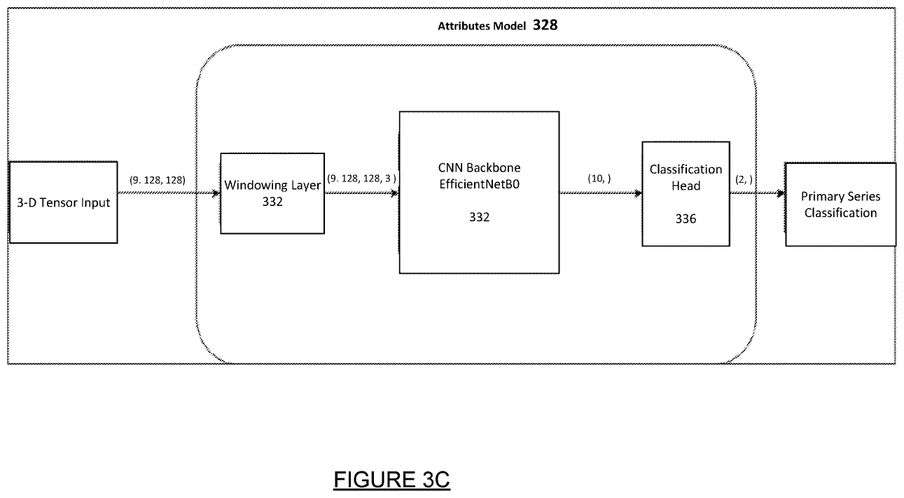

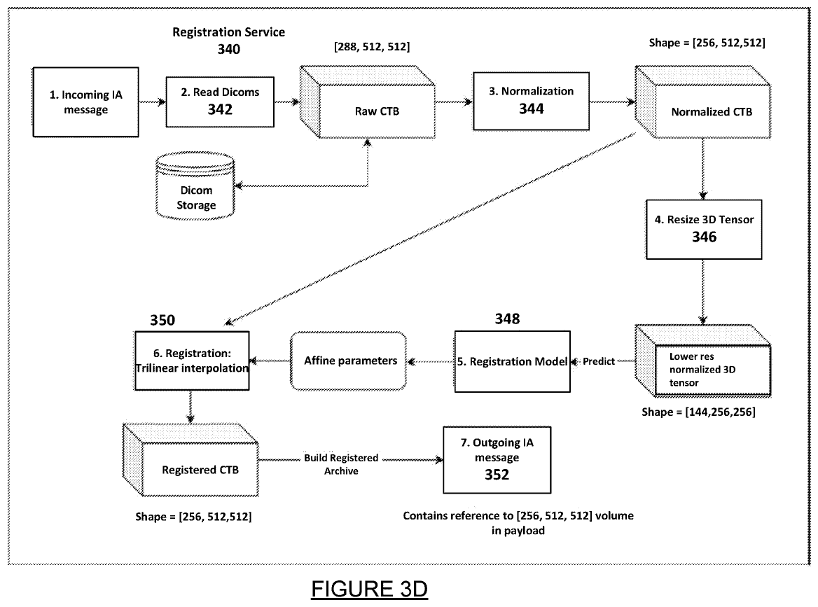

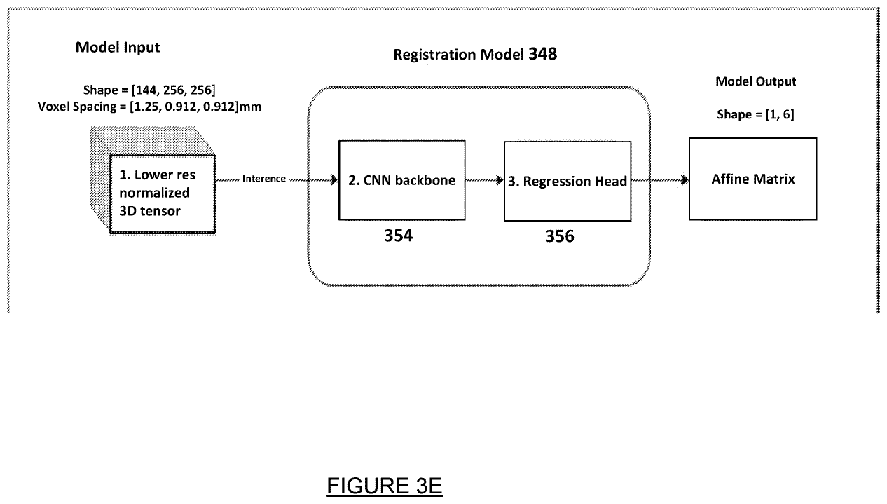

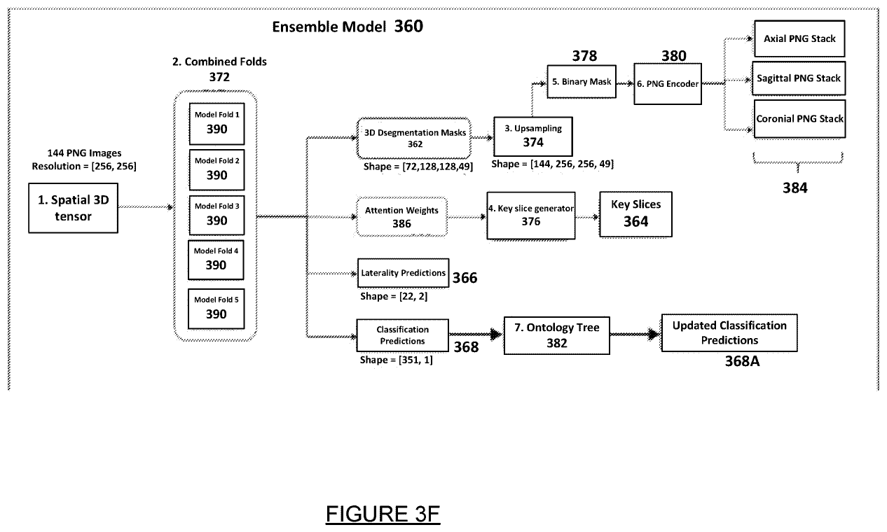

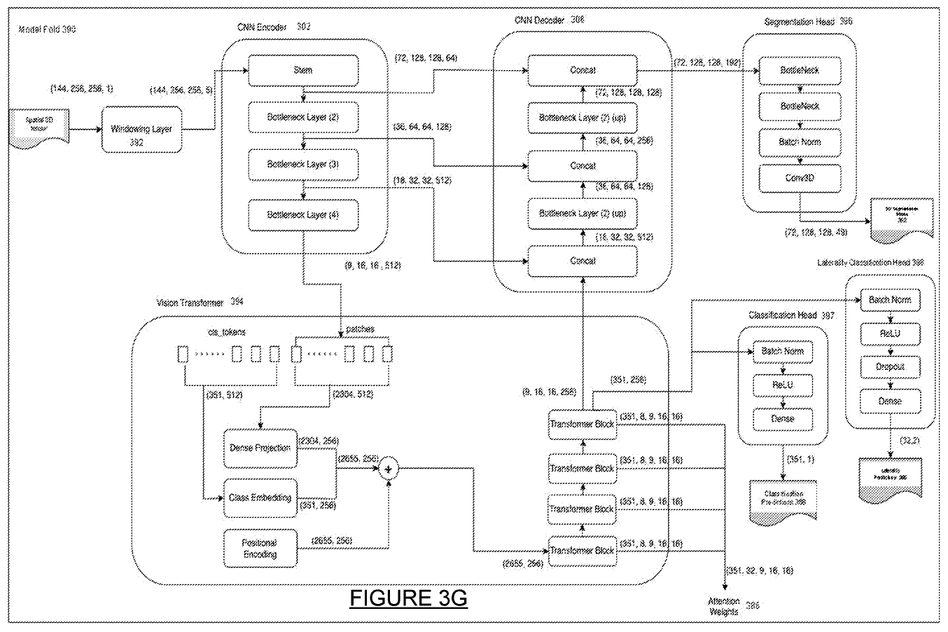

In some embodiments, as illustrated in FIG. 3A, a CNN model 200 comprises a CNN encoder 302 that is configured to process input anatomical images 304. The CNN encoder 302 functions as an encoder, and is connected to a CNN decoder 306 which functions as a decoder. At least one MAP 308 comprises a two-dimensional array of values representing a probability that the corresponding pixel of an anatomical slice, e.g. the input anatomical images 204, which exhibits a visual finding, e.g. as identified by the CNN model 200. The MAP 308 can be in a particular anatomical plane, and can be overlaid over a respective anatomical image 204 in the same anatomical plane. The anatomical image 204 may also be a 2D anatomical slice in the anatomical plane generated from a 3D spatial model of the subject. Additionally, a further classification output 310 may be provided, e.g. to identify laterality for a particular respective visual finding. The laterality identifies whether a visual finding is on the left versus the right side of the brain.

In example embodiments, a set of possible visual findings may be determined according to an ontology tree for CT brain as outlined in Table 1. These may be organized into a hierarchical structure or nested structure. Sublevels in a hierarchical ontology tree may be combined into more generalized findings (e.g. soft tissue haematoma being the parent class of neck haematoma and scalp haematoma). The generalized findings may be depicted as generic higher levels. The generalized findings are also evaluated as radiological findings in their own right.

Exemplary visual radiological findings for images 204 of a CT scan include those listed in Table 1. The use of a hierarchical structure for the set of visual findings may lead to an improved accuracy of prediction as various levels of granularity of findings can be simultaneously captured, with increasing confidence when going up the hierarchical structure.

The CT brain radiological findings ontology tree depicted in Table 1 was developed by a consensus of three subspecialist neuroradiologists.

An example ontology tree in Table 1 illustrates two hierarchal levels, called internal nodes and terminal leaves. Each pair of internal node and terminal leaf is denoted a pair or hierarchal pair. In examples, there are more than two hierarchal levels. In examples, each branch of hierarchal correlations can be denoted a branch, hierarchal branch, or hierarchal set.

The headings in the ontology tree of Table 1 can include any or all of the following:
leaf_id—classification class ID from ontology tree.
class_id—classification class ID used in product and training (same as leaf_id apart from fracture_c1-2 and intraaxial_lesion_csf_cyst).
Leaf Label—is the full finding name.
Definition—of finding as per user guide.
Localisation—segmentation or laterality. Defines how the localisation is applied.
laterality_id—laterality class ID used in product (optional mapping from class/leaf to laterality).
segmentation_id—segmentation class ID used in product and training (optional mapping from class/leaf to segmentation).
UI Segmentation Parent Name—segmentation parent name as displayed in UI.
UI Child Display Name—child name as displayed in UI.
ground_truth_id—ground truth.
Display Order—on finding level based on clinical importance. Parent display order can be inferred from the finding order.
group_id (1=Priority; 2=Other; 3=Tech)—used in triage and in UI.
Parent_Id—identifies the parent node.
slice_method—method used to determine default slice for display {Segmentation, Heatmap, Default}. IF slice_method is DEFAULT, then slice_axial, slice_coronal, slice_sagittal must be filled.
default_window—default window for display {Bone, Brain, Soft_tissue, Stroke, Subdural}.
slice_axial—default slice index for axial plane (only defined when slice_method is Default).
slice_coronal—default slice index for coronal plan (only defined when slice_method is Default).
slice_sagittal—default slice index for sagittal plane (only defined when slice_method is Default).
default planw—default plane for display {Axial, Coronal, Sagittal}.
enabled—used to turn findings on/off based on clinical review of multi-reader multi-case (MRMC) study results for initial release and model comparison results for subsequent releases (input for configurations). Used to construct list of findings for which thresholds will be calculated.
f_beta—used to compute thresholds based on clinically specified precision-recall tradeoff. Recall is Beta times more important than precision. Where Beta=1, precision and recall are equally important. In an example, this happens during deployment. Thresholds file is saved during deployment so it can be added to the configurations repo.
min_precision—used when calculating thresholds to ensure precision at optimal threshold is not too low to be clinically useful.
Is ontology parent—was defined in ontology tree as parent, but not a parent for display.
MRMC finding—included in the MRMC study (detailed below).

Particular pairs of data from Table 1 can be used by the system 100, for example when a particular visual anomaly finding (leaf_id or leaf label) is classified as being present, a higher hierarchal visual anomaly finding (parent_id) is also classified or re-classified as being present. When a particular visual anomaly finding is classified as being present, a default view such as a segmentation view, particular anatomical slice, segmentation mask, segmentation map, and/or laterality can be generated for display according to Table 1.

In an example, any one pair, such an internal node and a leaf node, in the hierarchical ontology tree in Table 1 can be classified by the CNN model 200. In another example, all of the possible visual anomaly findings listed in Table 1 can be classified by the CNN model 200.

Generally, in example embodiments, the images 204 of the CTBs are preprocessed into spatial 3D tensors of floats in Hounsfield units. In an example, the spatial 3D tensors are stored as a set of approximately 500 individual DICOM files. Below is described the process of building these spatial 3D tensors. Furthermore, the registration process needs to be aware of the geometry of the CTB (pixel spacing, shape, etc.), to help keep track this information the CNN model 200 generates the spatial 3D tensor. The spatial 3D tensor stores a 3D tensor of voxels plus geometric information. Generating the spatial 3D tensor includes the following.

Generating the spatial 3D tensor includes reading the DICOM (.dcm) files. For example, the package pydicom is used to read the individual .dcm files. The output of this step is a list of unsorted pydicom datasets.

Generating the spatial 3D tensor includes filtering. For example, the preprocessing layer removes any DICOM files that do not belong to the CTB by excluding any those that do not have the most common shape and orientation.

Generating the spatial 3D tensor includes sorting. For example, the preprocessing layer sorts the list of DICOM data by the z-position of each file, specifically, the 2nd element of the Imagine Patient Position metadata field.

Generating the spatial 3D tensor includes check spacing. For example, the preprocessing layer checks that the plurality of DICOM files result in a full CTB volume by checking if there are any gaps within the z-position of the Imagine Patient Position. The preprocessing layer defines the DICOM set as being invalid if there is a gap in the z-position that is larger than the mode of deltas between DICOMs: where delta=z_position_dicom_N−z_position_dicom_N+1.

Generating the spatial 3D tensor includes recalibrate. For example, the preprocessing layer recalibrates the voxel values based on the linear model with the Rescale Slope and Rescale Intercept headers from the DICOM image metadata.

Generating the spatial 3D tensor includes creating the spatial 3D tensor. For example, the preprocessing layer extracts the origin, spacing, direction, and shape of the input CTB (plurality of anatomical images) from both the DICOM image metadata and the DICOM image itself.

In an example, the CNN model 200 is used to perform a process or method for visual detection. The method includes receiving a series of anatomical images 204 obtained from a computed tomography (CT) scan of a head of a subject. The method includes generating, using the series of anatomical images by a preprocessing layer (in the integration layer 702): a spatial 3D tensor which represents a 3D spatial model of the head of the subject. The method includes generating, using the spatial 3D tensor by the CNN model 200: at least one 3D feature tensor. The method includes classifying, using at least one of the 3D feature tensors by the CNN model 200: each of a plurality of possible visual anomaly findings as being present versus absent, the plurality of possible visual anomaly findings having a hierarchal relationship based on a hierarchical ontology tree.

In an example, the system 100 generates for display on the viewer component 701 the plurality of possible visual anomaly findings classified as being present by the CNN model 200 in the hierarchal relationship defined by the hierarchical ontology tree. For example, the viewer component 701 can display a hierarchal relationship or hierarchal pair of the possible visual anomaly findings classified as being present by way of a tree layout, nesting, upper and lower, etc.

Referring to FIG. 3B, an attributes service 320 will now be described in greater detail. The attributes service 320 sits inside the integration layer 702 (also called integration adapter). The integration layer 702 is or includes a preprocessing layer. The integration adapter is or includes a preprocessing adaptor. The purpose of the attributes service 320 is to determine whether the input DICOM set is a non-contrast CT Brain. Anatomical images that are from a non-CT head scan are denoted “primary series”. The integration adapter will receive CTs from the PACs system based on the routing rules the attributes service 320 is used to filter out the CTBs, the filtered set of CTBs then get registered then sent to the CNN model 200.

The attributes service 320 in FIG. 3B includes the following modules and features.

Incoming IA Message 322: The incoming message contains the path to the CTB primary series.

Read DICOM Files 324: The raw DICOM files are converted in a 3D Tensor of float values.

Slice Selection 326 and resize: From the 3D tensor the attributes service 320 first resizes the volume to shape=[72, 128, 128] then nine slices are selected by uniformly sampling across the z-dimension. The final output of this phase is a tensor of size [9, 128, 128].

Attributes Model 328: The output Tensor from the slice selection 326 module is passed into the attributes model. The attributes model 328 has two modes: Thick-slice or Thin-slice. The set model in example implementations is Thin-Slice. The attributes model 328 will return a float value from 0 to 1. When this value is greater than the pre-set threshold, the CTB is considered a primary series.

Outgoing message 330: The outgoing message 330 contains, among other things, a Boolean flag that indicates whether the input series is primary or not, the attributes model score, and the attributes model version and code version.

Reference is now made to FIG. 3C, which illustrates an example of the attributes model 328 in greater detail. The attributes model 328 classifies the series of anatomical images 204 as primary or non-primary. The input to the attributes model 328 is the selected slices of the 3D volume (3D tensor input of size [9, 128, 128]). The 3D tensor input is the sliced 3D tensor of shape [9, 128, 128] with voxel spacing=[1.25, 0.912, 0.912] mm. The voxel values are in Hounsfield units.

Windowing Layer 332: The input 3-D tensor is separated into 3 channels by taking 3 windowed Hounsfield ranges.

CNN Backbone 334 (EfficientNetB0): The CNN Backbone 334 uses a EfficientNetB0 architecture to encode the 9 sampled slices.

Classification Head 336: The classification head 336 classifies the 3D tensor as being primary series versus not primary series.

Reference is now made to FIG. 3D, which illustrates a registration service 340. The registration service 340 sits inside the integration layer 702, which includes a preprocessing layer. The purpose of the registration service 340 is to create a standardized input for the CTB Ensemble model. The standardized input to the CTB Ensemble model is a Resolution=128, 256, 256 registered CTB brain.

The input to the registration service 340 is a message containing the location of a set of DICOM files saved in a MinlO file system, the DICOM files are for a non-contrast CT Brain. The output message from the registration service 340 is the path to an artifact generated in this service named a Registered Archive. The Registered Archive contains the registered CT Brain (in the form of a list of PNGs) and the spatial properties of the original and registered tensor.

Incoming message: the incoming message contains the path to the raw DICOM files.

Read DICOMs 342: the raw DICOM files are read and processed into a spatial 3D tensor. The spatial 3D tensor contains a 3D tensor of size, for example, [288, 512, 512], and the metadata associated with the Image Plane. Note the size of all these dimensions can vary in other examples.

Normalization 344: the registration service 340 uses trilinear interpolation to transform the pixel spacing and shape of the input spatial 3D tensors (3D volumes). The standardized values are shape=[256, 512, 512] and Pixel Spacing=[0.625, 0.912, 0.912] mm. The normalization 344 also standardizes the image orientation to the values defined in a template reference CTB.

Resize Tensor 346: The registration model 348 expects an input of resolution [128, 256, 256], therefore the resize tensor 346 model down-samples the image by a factor of two to ensure the input shape is correct. The result is a lower resolution normalized 3D tensor (of the CTB).

Registration Model 348: the registration model 348 makes an inference call using the [128, 256, 256] CTB tensor. The output is an affine matrix that will register with the input CTB. Specifically, the registration model 348 applies apply a set of rotations and translations to ensure the CT Brain has the same orientation as the reference CT template.

Registration 350: using the origin full resolution CT, and the affine transform matrix (affine parameters) from the model, the registration 350 module registers the CT Brain using trilinear interpolation.

Outgoing message 352: the registered CTB along with a collection of metadata is packaged inside a .NPZ file (this artifact is known as the registered archive). The registered archive is saved to the MinlO file system and the output message from the registration service contains the path to this artifact.

FIG. 3E illustrates the registration model 348 in greater detail. The function of the registration model 238 is to register CTB images 204 to a template CTB. Registering means to align (via rotation and translation) the CTB images 204 to the template CTB. The model does this by taking the CT images 204 as input and predicting the required affine matrix to register the CTB images 204.

Input data: the input to the registration model 348 is a CT Brain 3D Tensor with the following properties: Shape=[144, 256, 256] and voxel spacing=[1.25, 0.912, 0.912] mm. The voxel values are in Hounsfield units.

CNN Backbone 354: the registration model 348 uses a 3D Convolution Neural Network to process the input volume into a vector representation. This 3D CNN is built from residual connector blocks.

Regression Head 356: the output of the CNN Backbone 354 is converted into 6 numbers: 3 representation rotations and 3 representation translations. This occurs via a standard densely connected NN layer. The output is an affine matrix of shape [1, 6].

Reference is now made to FIG. 3F, which illustrates an ensemble model 360 of the CNN model 200. In an example, the ensemble model 360 creates a single model from the 5 individually trained model folds 390 (one for each validation fold). In an example, each fold 390 includes an equal number of randomly assigned input images 204 without the primary key (for example, patient ID) being in multiple folds to avoid data leakage. In an example, five model folds 390 were trained per project, one for each model fold 390 being the validation set (and the remaining model folds 390 being the training set), and later ensembled and postprocessed.

Furthermore, the ensemble model 360 includes several post-processing layers to transform the output of the ensemble model 360 into a more convenient representation. The ensemble model 360 outputs the following modules or functions:

3D segmentation masks 362: used to generate the axial, sagittal, and coronal viewpoints (each view is saved separately as a list of PNGs 384).

Key slice 364: derived from the attention weights 386 in the vision transformer 394 (also called ViT) (FIG. 3G) part of the CNN model 200.

The laterality predictions 366 identify whether a certain visual anomaly found to be present by the combined folds 372 is on the left or right side of the head. For example, up to 32 of the certain laterality predictions can be made. In another example, for the MRMC study detailed below, the CNN model 200 was used for 14 laterality predictions of certain classifications of the potential visual anomaly findings.

The classification predictions 368 are classifications of whether certain possible visual anomaly findings are found to be present versus absent.

An ontology tree module 382 uses the ontology tree to update any of the classification predictions 368 into updated classification predictions 368A. For example, using the hierarchical ontology tree, when a first possible visual anomaly finding at a first hierarchal level of the hierarchical ontology tree is classified in the classification predictions 368 by the combined folds 372 of the CNN model 200 to be present and a second possible visual anomaly finding at a second hierarchal level of the hierarchical ontology tree that is higher than the first hierarchal level is classified in the classification predictions 368 by the combined folds 372 of the CNN model 200 to be absent, the ontology tree module 382 re-classifies (modifies the classification of) the second possible visual anomaly finding to being present.

In an example, the method includes modifying, using the hierarchical ontology tree, when a first possible visual anomaly finding at a first hierarchal level of the hierarchical ontology tree is classified by the CNN model 200 to be present and a second possible visual anomaly finding at a second hierarchal level of the hierarchical ontology tree that is higher than the first hierarchal level is classified by the CNN model 200 to be absent, the classifying of the second possible visual anomaly finding to being present.

In an example, a training of the CNN model can be updated using the series of anatomical images labelled with the first possible visual anomaly finding as being present and the second possible visual anomaly finding as being present.

The input to the ensemble model 360 is a stack of 144 PNG images of resolution 256 by 256. These are registered, “primary series”, spatial representation of a CTB scan. The input is the spatial 3D tensor representing the head of the patient.

Combined Folds 372: The ensemble model 360 includes combined folds 372 which is a single model from the five model folds 390 which averages all of the model outputs from the five model folds 390.

Upsampling 374: The ensemble model 360 outputs segmentation at a resolution of [72, 126, 126] whereas the displayed CTB on the viewer component 701 has resolution [144, 256, 256]. Therefore, to overlay these two volumes the upsampling 374 module increases the resolution of the 3D segmentation masks 362. The resolution is increased by having the upsampling 374 module which repeats the data in the rows and columns by a factor of two.

Key Slice Generator 376: Using the attentions weights 386 (having attention values) from the vision transformer 394 layer (illustrated in detail in FIG. 3G) within the CTB model the key slice generation 376 module generates a set of key slices 364. The key slice 364 is set to the slice with the largest attention values.

Binary Segmentation Mask 378: the binary segmentation mask 378 outputs raw values from 0 to 1 for each segmentation, which are converted from these 3D float Tensors into Boolean values by thresholding at threshold=0.5.

PNG Encoder 380: the PNG encoder 380 converts the segmentation predictions into three sets of lists of PNGS 384 one list for each viewpoint (anatomical planes): axial, sagittal, and coronal.

Reference is now made to FIG. 3G, which illustrates an example of one model fold 390, in accordance with an example embodiment.

The model fold 390 processes a registered 3D input volume (spatial 3D tensor or input 3D tensor) and produces classification, segmentation, and classification findings. The model fold 390 also generates attention weights 386 which are used to generate (from the spatial 3D tensor) or select one of key slice 364 or default view of a particular anatomical slice of interest. The key slice 364 with the most attention weights is shown in the default view in the viewer component 701.

The input 3D Tensor is the registered 3-D float tensor of shape [144, 256, 256, 1] with voxel spacing=[1.25, 0.912, 0.912] mm. The voxel values are in Hounsfield units.

Windowing Layer 392: the input 3-D tensor is separated into 5 channels by taking 5 windowed Hounsfield ranges. The output is windowed spatial 3D tensors in a tensor of size [144, 256, 256, 5].

CNN Encoder 302: the CNN encoder 302 down-samples and encodes the spatial information of the input tensor. The CNN encoder 302 uses an adapted version of the ResNet model consisting of an initial stem along with BottleNeck layers which are built from convolutional layers. The input [144, 256, 256,5] tensor is downsampled whilst increasing filters to the last output shape of [9, 16, 16, 512]. The output from the CNN encoder 302 is one or more 3D feature tensors.

Vision Transformer 394 (ViT): the encoded output of the CNN encoder 302 is flattened and used as embedded patches of the original input 3D tensor. The sequential transformer layers incorporate attentional mechanisms using the embedded patches as inputs. The vision transformer takes in the [9, 16, 16, 512] input from the CNN encoder and has three outputs: a [9, 16, 16, 256) output corresponding to the CNN input, a [351, 256] output corresponding to 351 different class classifications, and a [351, 32, 9, 16, 16] output corresponding to attention weights for selecting the key slice or default slice. There can be more or fewer than 351 different class classifications in other examples. The attention weights are a stack of attention maps for particular visual anomaly findings found to be present in the input 3D tensor. The attention weights are in the form of an attention weight 3D tensor.

CNN Decoder 306: a U-Net architecture is used to decode and up-sample outputs from the vision transformer 394 as well as the CNN encoder 302 at various semantic levels through skip connections and concatenations.

Segmentation Head 396: the [72, 128, 128, 192] output from the CNN decoder 306 passes through the segmentation head 396 which contains several bottleneck layers and finally a 3D convolutional layer with 49 filters corresponding to the 49 segmentation findings. In a example, for the MRMC study detailed below, the CNN model 200 was trained for 49 segmentation predictions of certain classifications of the potential visual anomaly findings. Table 1 lists up to 66 segmentation findings, some of which are duplicated for certain classifications of the potential visual anomaly findings. In other words, one segmentation finding can be associated with many classification mapping. See, for example, Table.

Classification Head 397: in an example, 351 class tokens used as an internal representation within the vision transformer 394 for each of the classification findings. These are processed through the vision transformer 394 as outputs of shape [351, 256]. The classification head uses batch normalisation, ReLu activation and a Dense layer with sigmoid activation to output 351 classification findings.

Laterality Classification Head 398: the same input to the classification head 397 is also used as an input to the laterality classification head 398 which uses batch norm, ReLU activation, dropout and Dense layer with sigmoid activation to output a vector with shape [32, 2] corresponding to 32 left/right laterality findings for particular visual anomaly findings that are found to be present.

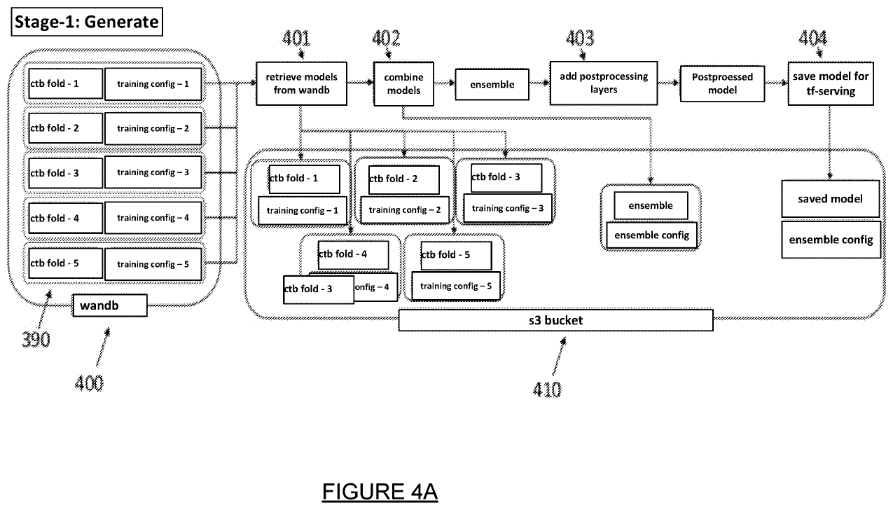

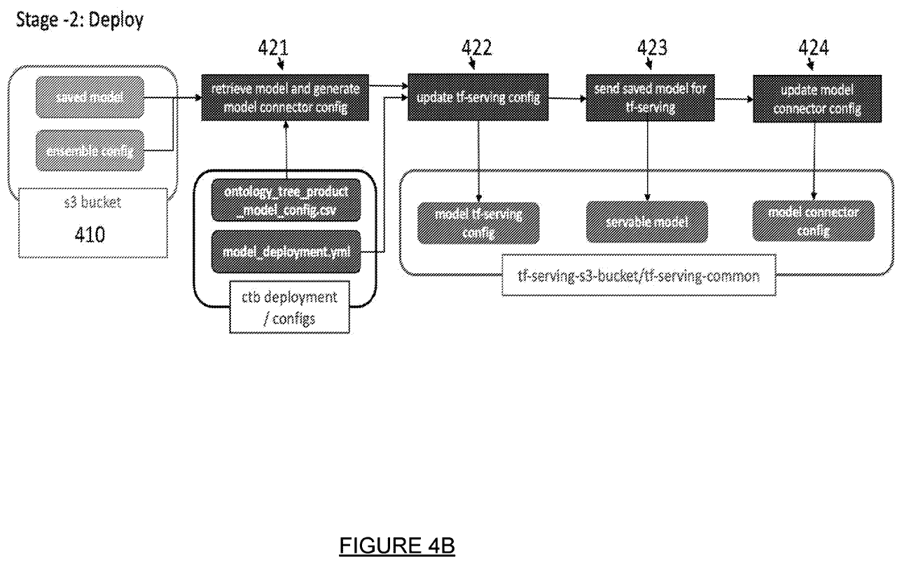

FIGS. 4A and 4B illustrate model generation and deployment, respectively. In this example, each model fold 390 represents a weight and bias (“wandb”) run 400 of the type included at https://wandb.ai/site. Each weight and bias run 400 contains the CNN diagram for that model fold 390 (also called “CTB fold”). For example, the model deployment process is initiated and executed as a Buildkite pipeline.

With reference to FIG. 4A, the following steps are run during the model generation stage:
1. Retrieve the model folds 390 of the CNN model 200 from the wandb runs (step 401) and persist in S3 storage unit (s3_bucket_name) (e.g., S3 from Amazon™) in the data store 108. If the same wandb run is used as more than one components, only one instance of that particular model fold 390 is saved.
2. Combine the model folds 390 into the ensemble model 360 (step 402).
3. Add pre- and post-processing layers to combined ensemble model 360 (step 403).
4. Convert combined ensemble model (CNN model 200) to tf (tensorflow)—serving model and persist in S3 storage unit in the data store 108 (step 404).

With reference to FIG. 4B, the following steps are run during the model deployment stage:
1. At step 410, retrieve CNN model 200, generate model connector configuration (step 421) and provide model server config to tf-serving S3 storage unit in the data store 108.
2. The tf-serving configured to automatically load the updated model server configuration from S3 (steps 422, 423).
3. Push model-specific connector configuration to a connector config database (step 424).

The model deployment process may be summarised as follows:
1. Update the model tf-serving configuration.
2. Update deployment configurations.
3. Run the model deployment.

At step 421 a model connector is generated to define a flexible integration layer 702 for system integration into the system 100 (see FIGS. 7A to 7C). The model connector includes classification, laterality and segmentation classes. In an example, the ontology tree of Table 1 includes all of the possible visual anomaly findings, a list of those possible visual anomaly findings flagged to check laterality, and a list of those possible visual anomaly findings flagged to generate the 3D segmentation mask. In one embodiment, a left-right (L/R) laterality model based on the attention-per-token ViT head (vision transformer 394) is provided to predict laterality during localization. In one example embodiment, laterality is part of localization which comprises segmentation outputs and laterality outputs.

The following post-processing layers are generated: i) Active slice calculation; ii) Class mappings; and iii) Key slice calculation.

These model outputs are then packaged into the return messages provided via the integration layer 702 comprising a CT connector, as described in detail herein with reference to FIGS. 7A to 7C.

In order to load a newly deployed model version, a tf-serving configuration is updated and saved. A list of historically updated models may be saved permanently. New model_version to be deployed must be listed under models->allowed_versions in this file and model_version_label must be specified along with the integer model version under models->version labels.

TensorFlowServing currently supports live updates of the model list but will only retrieve a new model if the version has been incremented. If model location in the s3 storage unit in the data store 108 remains the same TensorFlowServing clients will not retrieve the updated model. This means simply replacing the model files on the s3 storage unit with updated versions is not sufficient.

Thus given the CNN model 200 stored in the data store 108, the system 100 can update the configuration file to add the new version configuration. After publishing the model into s3 the config file (an exemplary excerpt of which, truncated, is provided below) to add a new version label and an additional version to the allowed policy:

```
model_config_list {
  config {
    name: ″ct-brain-png″
    base_path: ″/home/python/app/models/ct-brain-png/versions″
    model_platform: ″tensorflow″
    version_labels {
      key: ″1.0.7″
      value: 1
    }
  }
  config {
    name: ″ct-brain-ensemble″
    base_path: ″/home/python/app/models/ct-brain-ensemble/versions″
    model_platform: ″tensorflow″
    version_labels {
      key: ″1.0.7″
      value: 1
    }
  }
}
```

This configuration allows calls that use the unique wandb defined model key to specify which version of the model to use. Using the specific version policy allows tight control around which version is permitted and as the users specify the model by key, the system can guarantee that the correct model is being used. If a new CNN model 200 was deployed into a previously existing location without exhaustively ensuring that all tensorflow components are restarted it could result in running requests against incorrect models. This will not only allow deployments to occur without interruption by only ever using an increasing globally unique model number but will also to validate the build chain and retain previous versions for any future investigation.

An example of a CNN model 200 (also referred to as a CTB model) including pre and post processing layers is provided as follows:
Number of batches=1
Input tensor shape:
   144×256×256×1
   Windowing inside the model
   Send PNGs
Number of classes per output type
   num_classification_classes=(e.g. up to 351 nodes)
   num_segmentation_classes=49
   num_segmentable_findings=The number of segmentation outputs for which the relevant classification finding was positive
   num_laterality_classes=32
   default_slices of format (axial, sagittal, coronal)
Output tensor shape:
   classification_output:
      [batch_size, Num_classification_classes, n_ensemble]—float
   laterality_output:
      [batch_size, num_laterality_classes, 2]—float
         The two numbers represent Left and Right in the Axial axis only [LEFT, RIGHT]
         Metadata will be needed to identify which outputs are meaningful
   default_segmentation_slices:
      [batch_size, num_segmentation_classes, 3]—int
         the channel dimension is interpreted as of the order Axial/Sagittal/Coronal
         default_slice is the slice with the largest sum of pixels for that label along each axis.
         Return −1 if label not found
      default slices are shown in UI (viewer component 701)
   segmentation_output:
      [batch_size, num_segmentable_findings, num_frames_per_finding]—string
         For axial only
         Trained model output: produces a 72×128×128 segmentation mask for each segmentation finding (for 49 pre-specified segmentation masks, the vector is 72×128×128×49)
         PNGEncoder post-processing output: 144 axial images at 256×256 for each segmentation finding
         8-bit PNG bytes
         This minimises postprocessing as the UI needs to be able to read the different images.
         Removes postprocessing if PNGs, otherwise need to convert pixels into coordinates if DICOMs.
         The PNGs should use the full range of their colour depth to ensure max visibility. e.g.
         8 bit PNGs should have values of 0 or 255
   In an example, the postprocessing layers include the following steps:
      Obtain sum per image per label per axis for segmentation masks
      Obtain slice index of largest sum (this is the default slice per label per axis)
      Get axial PNGs

In an example, the following postprocessing layers are included for segmentation: i) Mask generator (via threshold); ii) Default slice calculator and non-empty image calculator; and iii) 3D tensor to tensor of PNG bytes.

For the purpose of training models in embodiments directed to analysis of images 204 of a CT scan (pixel data) a de-identified dataset of images 204 of a CT scan may be used. A sub-dataset including solely of anatomical images 204 of a CT scan is for example used for radiological findings. In this example, each image 204 of a CT scan is an NCCTB image obtained from a private radiology practice in Australia associated with a set of labels manually annotated by expert radiologists.

Inclusion criteria for the training dataset were: age greater than 16 years. Selected cases were from inpatient, outpatient and emergency settings. Data from all sources was de-identified. DICOM tags were removed. Protected health information was removed from reports and images through an automated de-identification process. Image data was preserved at the original resolution and bit-depth. Patient IDs and Study IDs were anonymised to de-identify them while retaining the temporal and logical association between studies and patients. The resulting dataset comprises 212,484 NCCTB images. The median number of model training cases per clinical/radiological finding in this dataset is 1380. This is the median count per label that's over all the labels used in training the CNN model 200.

Thirty-two radiologists each reviewed 2,848 NCCTB cases in a test dataset with and without the assistance of the deep learning model at different times. Ground truth labels were obtained from the consensus of three subspecialist neuroradiologists with access to reports and clinical history.

All participants in the labelling and evaluation phases were trained to identify the clinical findings according to the ontology tree shown in Table 1.

Each of the 212,484 NCCTB images was independently labelled by three radiologists randomly selected from a pool of 120 radiologists. Case order was randomised and placed in a queue. As radiologists completed each case, they were allocated the next incomplete case that they had not yet labelled according to the queue order. This process ensured that each case was labelled by three different radiologists. Each radiologist was provided with the same data for each case but was blinded to the labels of the other two radiologists. The de-identified radiology report, age and sex were provided, along with all NCCTB series in the study (including contrast scans), and paired CT/MRI scans close in time to the NCCTB case of interest.

Labels included of classification labels on a case level, indicating whether each finding was “present” versus “absent” in the case, as well as a 3D segmentation and laterality for certain relevant findings. The consensus for each finding for each triple-read cases was generated as a score between 0 and 1 using the Dawid-Skene consensus algorithm, which accounts for the relative accuracies of each labeller for each finding. Segmentation maps were generated by a single radiologist to visually localize (drawing, outlining, or making a rectangle through the viewer component 701) the pathology and were used to train the model to produce overlay outputs. The radiologist can also indicate laterality (left side versus right side).

Performance metrics including area under the receiver operating characteristics curve (AUC) were calculated and average radiologist performance metrics were compared to those of the model for each clinical finding, as will be detailed in the study described below.

An example method of training the convolution neural network (CNN) model 200 includes: generating a user interface (for display on the viewer component 701) including a labelling tool for a plurality of sample CT images from one or more CT scans/studies. The labelling tool allows at least one expert to select possible visual anomaly labels as being present versus absent presented in a hierarchical menu which displays the possible visual anomaly findings in a hierarchal relationship from a hierarchical ontology tree. Through the hierarchical menu, labelling of a first possible visual anomaly label at a first hierarchal level of the hierarchical ontology tree as being present in at least one of the sample CT images automatically labels a second possible visual anomaly label at a second hierarchal level of the hierarchical ontology tree that is higher than the first hierarchal level as being present in the sample CT images. The method includes receiving, through the user interface, the selected possible visual anomaly labels for each of the CT scans. The method includes training the CNN model 200 using the plurality of sample CT scans labelled with the selected labels, including the first possible visual anomaly label as being present and the second possible visual anomaly label as being present.

In an example inference performed by the CNN model 200, the CNN model 200 can classify, using the plurality of sample CT images by the CNN model 200: each of the possible visual anomaly findings as being present versus absent.

In an example, the labelling tool with each of the plurality of sample CT images can be displayed with the possible visual anomaly findings classified as being present versus absent by the CNN model 200, to assist a user in re-labelling through the labelling tool of the sample CT images with second possible visual anomaly label as being present versus absent by the at least one expert. The system 100 can receive, through the labelling tool of the user interface, the second possible visual anomaly label for the CT scans.

In an example during inference of anatomical images 204 of a CT scan, the CNN model 200 may initially classify the first possible visual anomaly label as being present and the second first possible visual anomaly label (having the high hierarchal level) as being absent. The second first possible visual anomaly label is considered to be a false negative by the CNN model 200. The CNN model 200 (or a processor of the system 100) can be configured to modify, using the hierarchical ontology tree, the classification of the second possible visual anomaly finding to being present. The method of training can include updating the training of the CNN model 200 using the anatomical images 204 labelled with the first possible visual anomaly finding as being present and the second possible visual anomaly finding as being present. Therefore, the CNN model 200 is re-trained to learn from the false negative with the benefit of the hierarchical ontology tree.

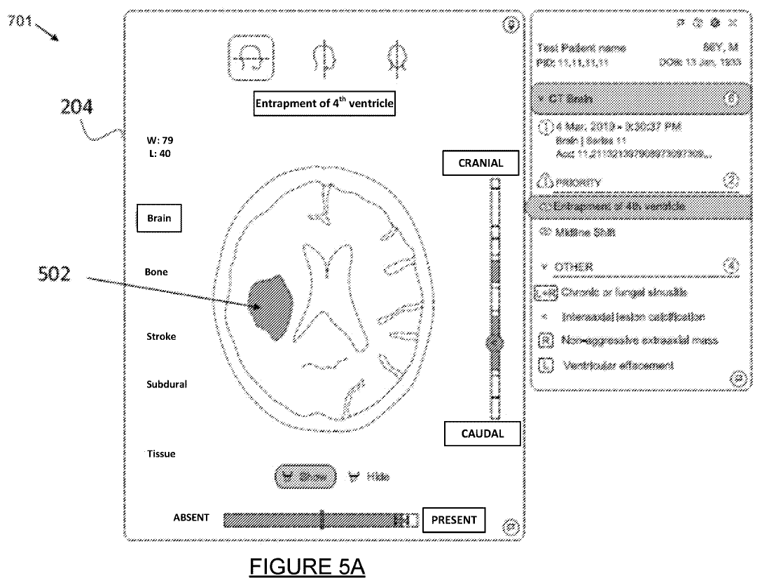

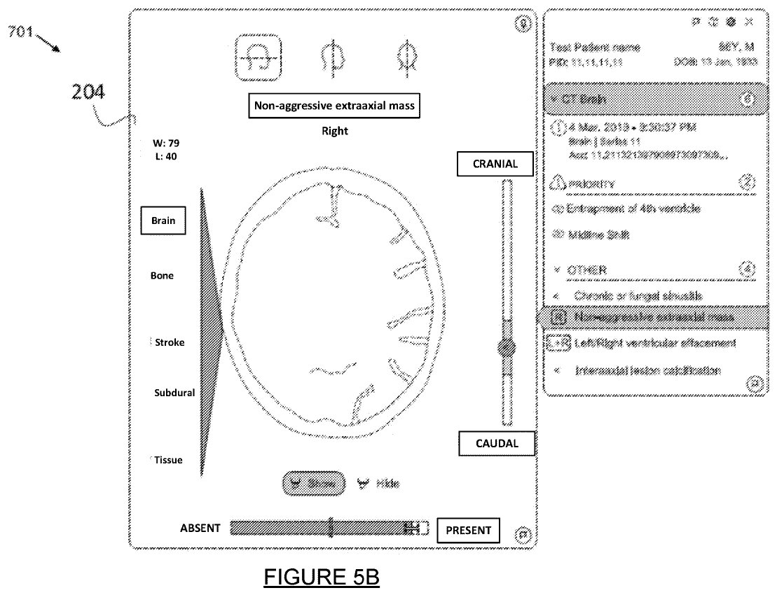

The output (e.g., MAP 308, classification output 310) of the CNN model 200 may provide additional context and positional information for a subset of the radiological findings of the CNN model 200. Predictions made by this deep learning model for a particular finding can be of one of the following forms:
No localization;
Segmentation MAP 308, as shown in FIG. 5A comprises a segmentation MAP 502 (mask/overlay) on top of one or more input images 204;
Laterality comprises a prediction of whether a finding is present in the left, right, or both (i.e. bilateral). With reference to FIG. 5B, the intensity of each side of the image 204 may be determined by the probability of the finding being in the left or right of the image 204; i.e. laterality.

An example of the laterality detected by the CNN model 200 for particular visual findings are outlined in Table 1. For example, when a particular visual anomaly finding is found to be present by the CNN model, then the particular left-right laterality is also determined by the CNN model 200 if the identifier of “laterality” is also indicated in Table 1.

From a user perspective, the display of findings is based on the classification findings (e.g. if the classification finding is not present or disabled, then no localization is displayed for that finding).

In an example of the output relating to images 204 of a CT scan, a finding may have laterality or segmentation.

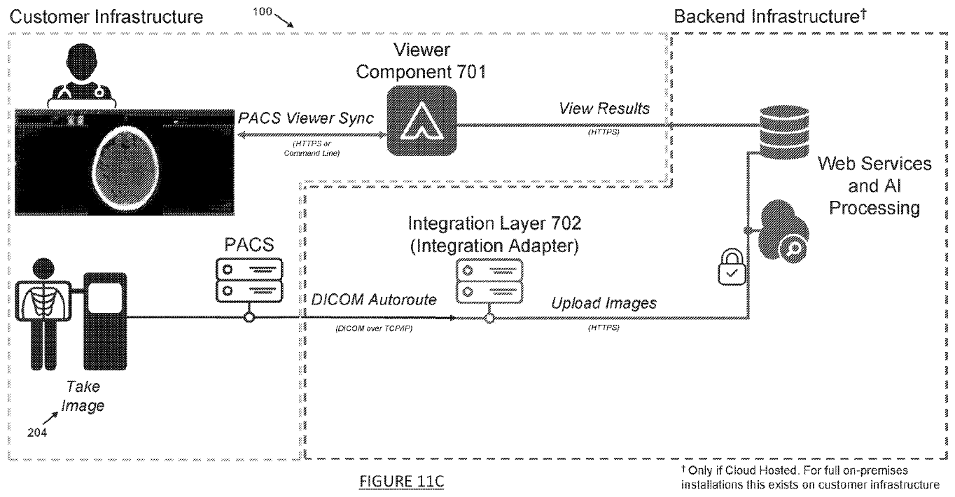

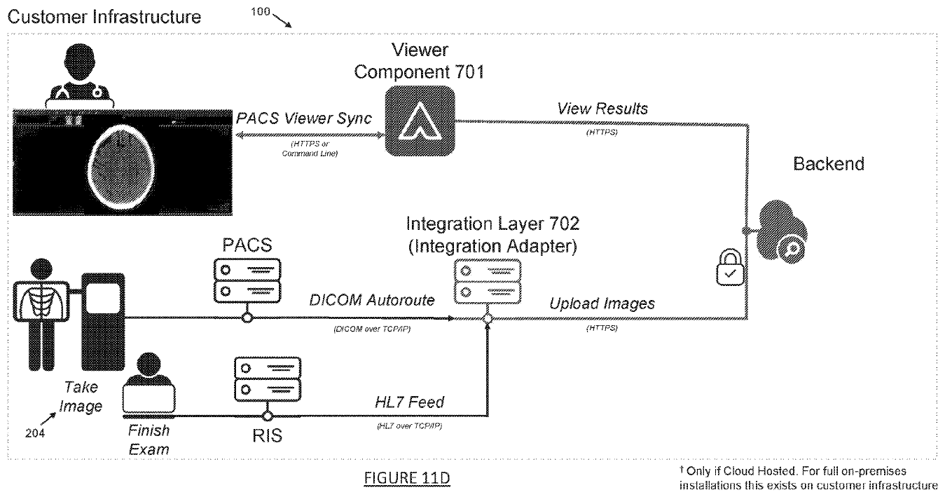

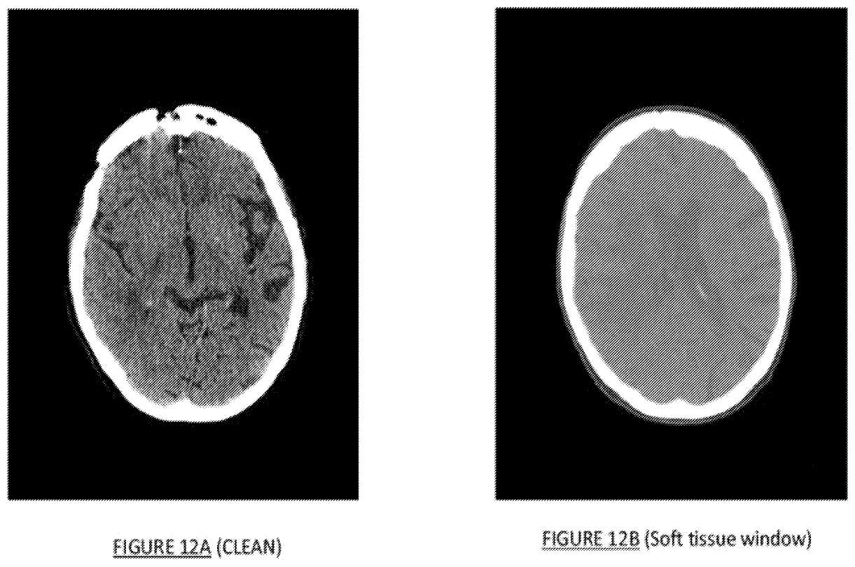

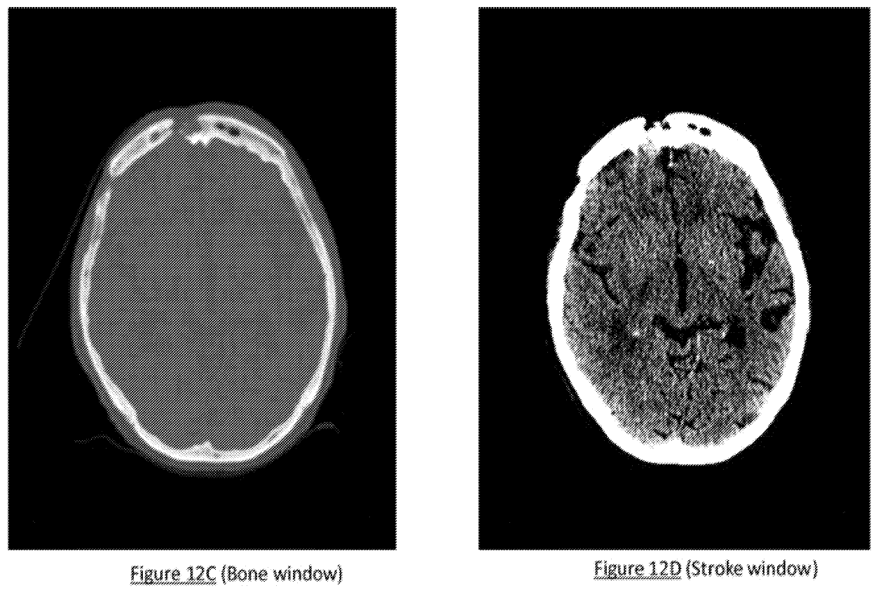

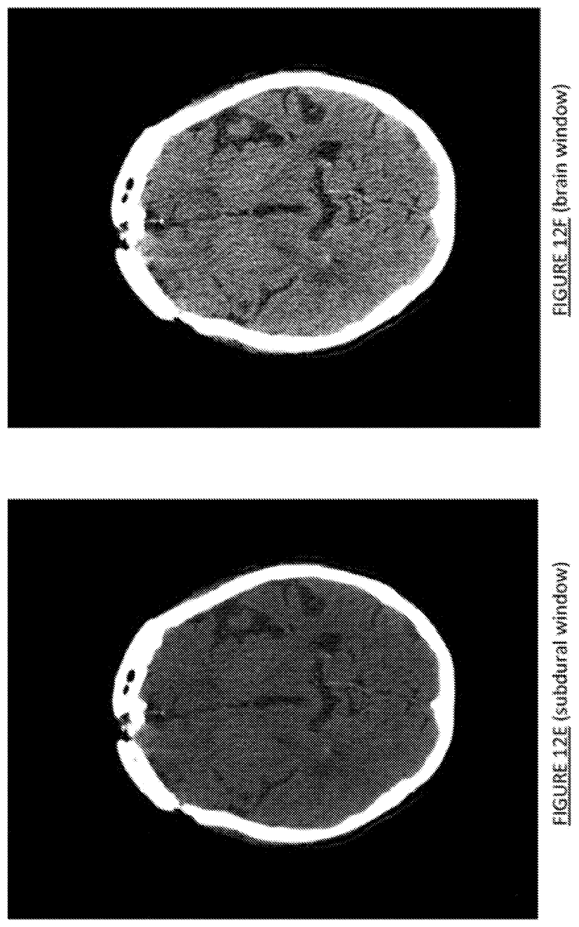

In the present example, images are displayed using one of 5 window presets (e.g. user interfaces or windowing types): soft tissue, bone, stroke, subdural or brain (see examples in FIGS. 12B to 12F, respectively, with the clean input image shown in FIG. 12A for comparison). The default window setting for each finding will be advantageously defined by the ontology tree of Table 1.

When a user selects a finding, the device shows the finding in a default projection. When the user switches projections, the system advantageously displays a “key slice”, representing the slice which the user ought to see first. The relevant fields of the ontology tree (Table 1) are slice_method, slice_axis, slice_coronal, and slice_sagittal.

The logic for the automated selection of the key slice for display is as follows:
if slice_method==Default then the hardcoded value indicated in slice_coronal, slice_sagittal, slice_transverse is used as the key slice, each referring to one of the 3 anatomical planes: sagittal, coronal, transverse.
else if slice_method==Segmentation then segmentation key slices. For example, the prominent segmentation slice along the axes, e.g. the slice with the highest area covered of all of the segmentation maps, the largest sum of pixels for that label along that axis of the anatomical plane, is used.
else if slice_method==Heatmap then the prominent segmentation slice along the axes in attention layer based heatmaps are used. For example, the highest total attention values along that axis of the anatomical plane.

In an example embodiment, the default/key slice is selected based on attention per class using the attention-per-token ViT head (vision transformer 394).

This enables providing an ideal default view to the user for the user to confirm the presence of a finding detected by the model by displaying the medical prediction in the easiest manner to the user rather than the user having to search through the plurality of slices through a plurality of anatomical planes through a plurality of windows/user interface. More specifically, the system is configured to select an optimal combination for an ideal default view, of: i) “slice-method” (e.g. which one of the 3 anatomical planes: sagittal, coronal, transverse)” is the best for the user to visually confirm the presence/absence of the radiological finding; ii) slice image 204 (which image 204 out of X number of the stack of images 204 of a CT scan) is the default (or best) for the user to visually confirm the presence/absence of the radiological finding; and iii) window to display the slice image (e.g. which window from the brain, bone, soft tissue, bone, stroke, subdural windowing presets) is the best for the user to visually confirm the presence/absence of the radiological finding.

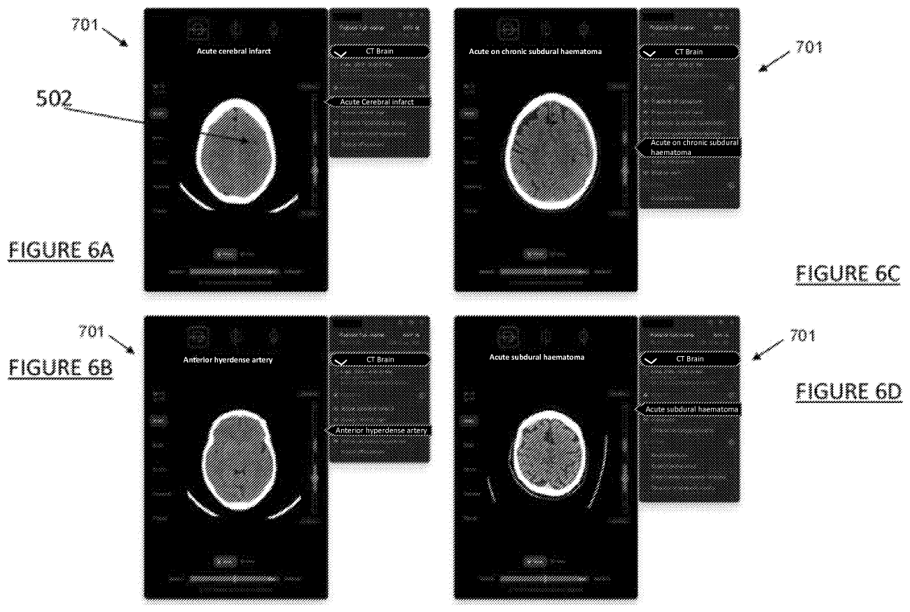

The default projection/view may be predefined as follows:

| For a finding | Default | |
|---|---|---|
| with: | Projection | Key Slice |
| CLASSIFICATION | defined in Table 1 | defined in the ontology tree per |
| | ontology tree per | finding and view (default is the |
| | finding | middle slice) |
| LATERALITY | use classification | use classification |
| | per Table 1 | |
| SEGMENTATION | use classification | Identified as the one with the |
| | per Table 1 | most colour (e.g. greatest total |
| | | area covered) in FIGS. 6A to 6D |

FIG. 6A to 6D show exemplary interactive user interface screens of the viewer component 701 in accordance with an example embodiment. Clinical findings detected by the deep-learning model are listed and a segmentation MAP 502 (identified as one with most colour e.g. purple) is presented in relevant pathological slices. Finding likelihood scores and confidence intervals are also displayed under the NCCTB scan, illustrated as a sliding scale at the bottom of absent versus present.

The UI addresses communicating AI confidence levels to a user (e.g. a radiologist) that is intuitive and easy to understand. In various example embodiments, the viewer component 701 may be implemented as web-based code executing in a browser, e.g. implemented in one or more of JavaScript, HTML5, WebAssembly or another client-side technology. Alternatively, the viewer component 701 may be implemented as part of a stand-alone application, executing on a personal computer, a portable device (such as a tablet PC), a radiology workstation 112, or other microprocessor-based hardware. The viewer component 701 can receive the results of analysis by the CNN model 200 of images 204 of a CT scan from, e.g., a RIAS 110 or an on-site radiology image analysis platform 114. Analysis results by the CNN model 200 may be provided in any suitable machine-readable format that can be processed by the viewer component 701, such as JavaScript Object Notation (JSON) format.

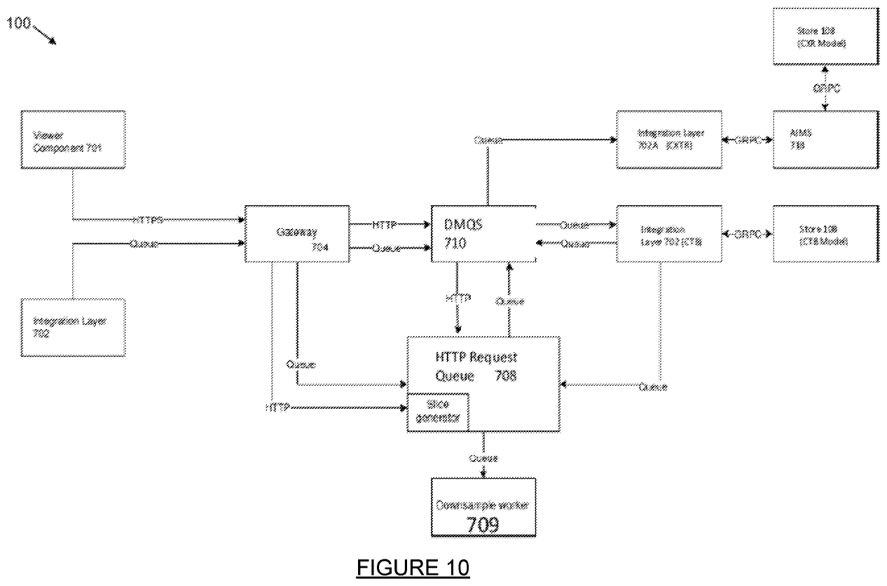

Referring to FIGS. 7A to 7C, FIG. 10, and FIGS. 11A to 11D, the exemplary system 100 for analyzing radiology images 204 will now be described. The exemplary system 100 is based on a microservices architecture, a block diagram of which is illustrated in FIG. 7A, and comprises modular components which make it highly configurable by users and radiologists in contrast to prior art systems which are rigid and inflexible and cannot be optimised for changes in disease prevalence and care settings. Another benefit of a modular systems architecture comprising asynchronous microservices is that it enables better re-usability, workload handling, and easier debugging processes (the separate modules are easier to test, implement or design). The system 100 also comprises modular components which enable multiple integration pathways to facilitate interoperability and deployment in various existing computing environments such as Radiology Information Systems Picture Archiving and Communication System (RIS-PACS) systems from various vendors and at different integration points such as via APIs or superimposing a virtual user interface element on the display device of the radiology terminals/workstations 112. FIG. 10 shows exemplary system with CTB capability. The virtual user interface element may be an interactive viewer component 701.

The system 100 includes a plurality of integration pathways via modular subcomponents including: PACS injection, RIS injections, synchronised viewer component 701, PACS inline frame (iFrame) support, PACS Native AI Support, or a Uniform Resource Locator (URL) hyperlink that re-directs the user to a web viewer on a web page executed in a web browser. The system 100 may comprise an integration layer 702, comprising one or more software components that may execute at on-premises hardware. The integration layer 702 may include a library module containing integration connectors, each corresponding to an integration pathway. Depending on the PACS system that is used by a customer, the library module may receive a request for a particular integration connector for the system 100 to interact with the customer via the PACS system. Similarly, depending on the RIS system that is used by a customer, the library module may receive a request for a particular integration connector for the system of the present example to interact with the customer via the RIS system, for triage injection for re-prioritisation of studies. Certain integration connectors occupy or block a large portion of the viewport and this may be undesirable in certain circumstances for users.

In one example, PACS Native AI Support is used as the integration connector (in the integration layer 702) because the PACS is configured to display medical predictions from the CNN model 200 example natively, and the user interface resembles the existing PACS system. For example, the PACS Native AI Support may have a plurality of Application Programming Interfaces (APIs) available that enable the system of the present example to communicate with such a PACS.

In another example, where a conventional radiology workstation 112 is unavailable or a PACS system is inaccessible, a user may use a mobile computing device such as handheld tablets or laptop to interact with the system of the present example by injecting a URL link in a results window of an electronic health record (EHR) that, when clicked by the user, causes an Internet browser to direct them to a web page that executes a web viewer application (viewer component 701) to display the image 204 of the CT scan and radiological findings predicted by the CNN model 200. The web viewer displays one or more of the image 204 of a CT scan with the segmentation indicated (overlaid) and the radiological (visual anomaly finding) classifications detected by the CNN model 200.

In another example, a synchronised viewer component 701 may be used as the integration connector (in the integration layer 702) to overlay on an existing PACS system that may lack APIs to enable native AI support. The viewer component 701 displays the image 204 of a CT scan with the radiological findings detected by a deep learning network, such as the CNN model 200. The viewer component 701 is repositionable by the user in the viewport in the event the viewer component 701 obscures the display of any useful information supplied from the PACS system.

The system 100 comprises modular user configuration components to enable users (e.g. clinicians and radiologists) to selectively configure the quantity of radiological findings they would like detected particular to their care setting. For triage injection the system 100 can configure priority for each finding and match that to a preference setting configured by the customer.

A microservice is responsible for acquiring data from the integration layer 702 to send images 204 of a CT scan to the CNN model 200 for generating predicted findings. The microservice is also responsible for storing study-related information, images 204 of a CT scan and AI result findings. The microservice provides various secure HTTP endpoints for the integration layer 702 and the viewer component to extract study information to fulfil their respective purposes. In an exemplary embodiment, image formats accepted by the microservice is JPEG2000 codestream lossless format. Other image formats are acceptable such as PNG and JPEG. The microservice validates all images 204 of a CT scan before they are saved and sent downstream for further processing.

The microservice functions (cloud-based or on-premises) may be summarised in the following workflow:
1. Receive study information from the integration layer 702:
   a. Receive images 204 of a CT scan from the integration layer 702
   b. Process and extract relevant study information and store into a database
   c. Store the images 204 of a CT scan into a secure blob storage or object storage 712 (for example, an S3 bucket in AWS for a cloud deployment)
2. Send images 204 of a CT scan to the CNN model 200:
   a. Receive a study is ready for AI processing message from the integration layer 702
   b. Prepare and transmit the images 204 of a CT scan to the CNN model 200 for generating predicted findings
   c. Store CNN model 200 generated predicted findings into a database
3. Receive request from viewer component 701:
   a. Send study information, images 204 of a CT scan and CNN model 200 generated predicted findings
4. Receive request from the integration layer 702:
   a. Send the relevant study with its images 204 for processing by the CNN model 200
   b. Send complete study with CNN model 200 generated predicted findings back to the integration layer.

With reference to FIG. 10, CTB slice images 204 may also be received from the integration layer 702A using a separate pre-processor microservice AIMS 718. In an example, the system 100 is customised to select an appropriate microservice (e.g. chest X-Ray (CXR) or CTB) so that the correct CNN model(s) 200 (e.g. relevant to CXR or CTB) is queried and served. In envisaged embodiments, a mixture of C×R images and images 204 of a CT scan may be processed at the same time when both separate microservices are in use as appropriate.

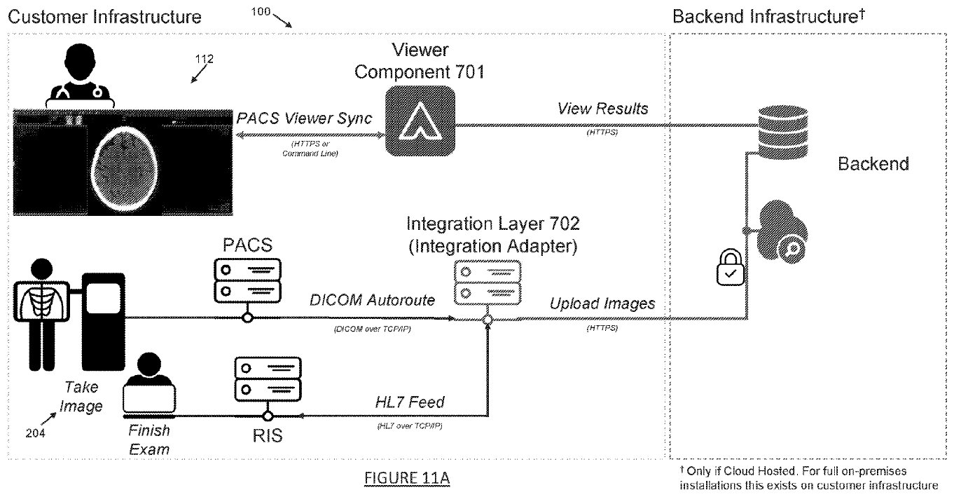

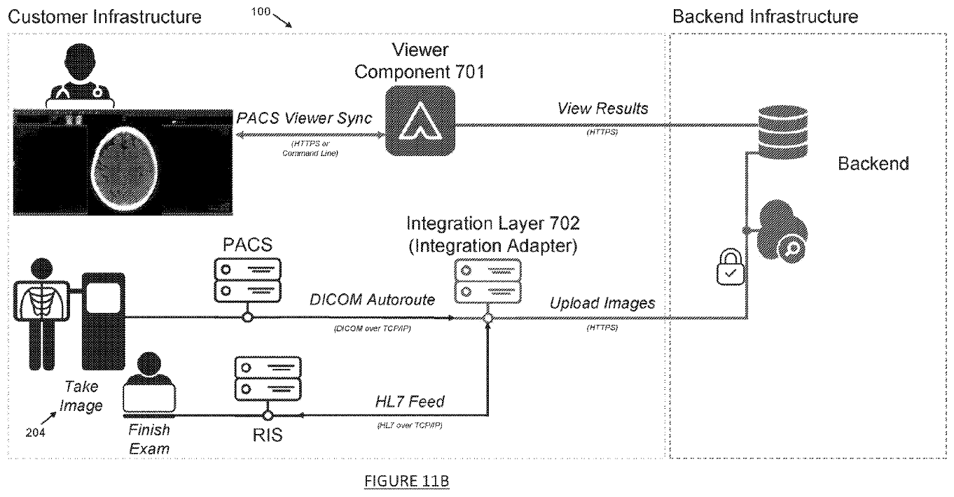

Various examples of the workflow are illustrated in FIGS. 11A to 11D, spanning from the step of inputting the images 204 of the CTB scan into the system 100 for the CNN model 200 to perform medical predictions to the step of presenting an output (viewer component 701 for the workstation 112) to the user.

The RIS system can be configured to track a patient's entire workflow within the system 100. The radiologist can add images 204 and reports to the backend, where the images 204 and reports can be retrieved by the CNN model 200 and also accessed by other radiologists and authorized parties.

The RIS system and the PACS system can be separate terminals in an example. In another example, the RIS system and the PACS system can be the same terminal. In examples, the viewer component 701 may be combined or separate with the RIS system and/or the PACS system.

In an exemplary embodiment the architecture of the microservice is an asynchronous microservices architecture. The microservice uses a queueing service. The queuing service in a cloud deployment may provided by a host cloud platform (for example, Amazon Web Services™, Google Cloud™ Platform or Microsoft Azure™) to transmit messages from one microservice to the next in a unidirectional pattern. The queuing service in an on-premise deployment may be a message-broker software application or message-oriented middleware, comprising an exchange server and gateways for establishing connections, confirming a recipient queue exists, sending messages and closing the connections when complete. Advantageously this arrangement enables each microservice component to have a small and narrowed function, which is decoupled as much as possible from all the other narrowed microservice functions that the microservice provides. The advantage of the microservices pattern is that each individual microservice component can be independently scaled as needed and mitigates against single points of failure. If an individual microservice components fail, then the failed microservice components can be restarted in isolation of the other properly working microservice components.

All microservices are, for example, implemented via containers (e.g. using Docker™, or a similar containerization platform). A container orchestration system (e.g. Kubernetes™, or similar) is, for example, deployed for automating application deployment, scaling, and management.

In an exemplary embodiment there is a single orchestration cluster with a single worker group. This worker group has multiple nodes, each of which may be a cloud-based virtual machine (VM) instance. After a microservice is deployed, the containers are not guaranteed to remain static. The orchestration system may shuffle containers depending on a variety of reasons. For example:
1. exceeding the resource limits and subsequently killed to avoid affecting other containers;
2. crashes may result in a new container spun up in a different node to replace the previous container;
3. to dynamically add or remove compute capacity based on an increase or decrease in workload/demand; and/or
4. an increase and then decrease in replicas can result in shift to a new node.

Referring to FIG. 7A, a gateway 704 provides a stable, versioned, and backward compatible interface to the viewer component 701 and the integration layer 702, comprising a CT model connector with a TFServing container per connector. There is a many to one relationship between the CT connector and the CT models (CNN models 200 for the CT scans).

The gateway 704 provides monitoring and security control, and functions as the entry point for all interactions with the microservice. The gateway 704 transmits images 204 of a CT scan to secure blob or object storage 712, and provides references to microservices downstream that require access to these images 204 of a CT scan. The gateway 704 is responsible for proxying HTTP requests to internal HTTP APIs and dispatching events into a HTTP Request Queue 708.

With reference to FIG. 10, the HTTP Request Queue 708 stores as a large Binary Large Object (blob), images 204 of a CT scan and segmentation map output. A blob consists of binary data stored as a single item, for example, image data or pixel data. A blob may be around 30 MB to 100 MB. The gateway 704 downsamples image input using a downsample worker unit 709. The downsample worker unit 709 is used to reduce the size of CT registered archive data (registered CTB images) to half scale for the mode as well as slice generation for presenting to the viewer component 701. Further, HTTP Request Queue 708 generates slices from segmentation and the downsample registered archive. The HTTP Request Queue 708 stores and validates tensor data, factoring in multiple versions of series to produce the tensor. The multiple versions are expected due to network drops or other disconnections.

An example registered archive format is as follows for the NPZ archive: png_list: 1d array containing the list of axial PNG's as bytes; png_offset; registered_spacing; registered_direction; registered_origin; original_spacing; original_direction; original_origin.

The HTTP Request Queue 708 generates view slices for the viewer component 701 from the downsampled registered archive. The HTTP Request Queue 708 further generates view slices for the viewer component 701 from the segmentation model processing results.

The gateway 704 splits the payload of messages between the HTTP Request Queue 708 and a distributed message queueing service (DMQS) 710. The DMQS 710 accepts incoming HTTP requests and uses a model handling service (MHS) 716. The DMQS 710 stores studies, images 204 of a CT scan, and deep learning predictions into a database managed by a database management service (DBMS) 714. The DMSQ 710 also manages each study's model findings state and stores the AI classification findings predicted by the CNN models 200, and stores errors when they occur in a database via the DBMS 714, accepts HTTP requests to send study data including model predictions for radiological findings, accepts HTTP requests to send the status of study findings, and forwards images 204 of a CT scan and related metadata to the MHS 716 for processing of the AI findings.

The DMQS 710 obtains classification and 3D localization outputs from the CT connector of the integration layer 702. It performs post processing of the 3D localization component and default slice information per label per axis from the integration layer 702 for segmentation masks.

An advantage of the DMQS 710 is that message queues can significantly simplify coding of decoupled applications, while improving performance, reliability and scalability. Other benefits of a distributed message queuing service include: security, durability, scalability, reliability and ability to customise.

A security benefit of the DMQS 710 is that who can send messages to and receive messages from a message queue is controlled. Server-side encryption (SSE) allows transmission of sensitive data (e.g. the image 204 of a CT scan) by protecting the contents of messages in queues using keys managed in an encryption key management service.

A durability benefit of the DMQS 710 is that messages are stored on multiple servers compared to standard queues and FIFO queues.

A scalability benefit of the DMQS 710 is that the queuing service can process each buffered request independently, scaling transparently to handle any load increases or spikes without any provisioning instructions.

A reliability benefit of the DMQS 710 is that the queuing service locks messages during processing, so that multiple senders can send and multiple receivers can receive messages at the same time.

Customisation of the DMQS 710 is possible because, for example, the messaging queues can have different default delay on a queue and can handle larger message content sizes by holding a pointer to a file object or splitting a large message into smaller messages.

Advantageously, the DMQS 710 is optimised for processing small messages/metadata, including classification predictions, laterality outputs and segments metadata.

The MHS 716 is configured to accept DICOM compatible images 204 of a CT scan and metadata from the DMQS 710. The MHS 716 is also configured to download images 204 of a CT scan from a cloud image processing service (CIPS) 706.

The AIMS 718 is configured as a pre-processor microservice that interfaces with the MLPS 720 and MHS 716. This modular microservices architecture has many advantages as outlined earlier. The AIMS 718, for example, communicates using a lightweight high-performance mechanism such as gRPC. The message payload returned by the AIMS 718 to MHS 716 contains predictions that include classifications and segmentations. These predictions are stored into a database by the DMQS 710 via DBMS 714.

The MLPS 720 is a containerized service comprising code and dependencies packaged to execute quickly and reliably from one computing environment to another. The MLPS 720 comprises a flexible, high-performance serving system for machine learning models, designed for production environments such as, for example, TensorFlow Serving. The MLPS 720 processes the images in the deep learning models and returns the resulting predictions to the AIMS 718. The CNN model 200 may be retrieved for execution from a cloud storage resource data store 108, such as a CTB model of the type described above which comprises a classification and segmentation model. The model is served in this example by a TFServing container in protobuff format. The MLPS 720 returns the model outputs (e.g. the predictions) to the AIMS 718.

The system 100 further includes a CIPS 706, which communicates at least with the gateway 704, and the MHS 716, as well as with the cloud storage 712. The primary functions of the CIPS 706 are to: handle image storage; handle image conversion; handle image manipulation; store image references and metadata to studies and findings; handle image type conversions and store the different image types, store segmentation image results from the CNN model(s) 200; manipulate segmentation PNGs by adding a transparent layer over black pixels; and provide open API endpoints for the viewer component 701 to request segmentation maps and images (in a compatible image format expected by the viewer component 701).

FIG. 7B illustrates a method (process and data transfers) for initiating AI processing of medical imaging study results, according to an exemplary embodiment. An image upload event notifies the microservice that a particular study requires generation of CNN model 200 finding results (e.g. predictions). The incoming request initiates saving of all relevant study information including the series, scan and image metadata into a secure database via the DBMS 714. The images 204 of a CT scan are also securely stored in cloud storage 712, for use later for the model processing.

In particular, at step 722 the integration layer 702 sends a request comprising an entire study, includes associated metadata, e.g. scan, series and images 204 of CT scans. The request is received by the gateway 704 which, at step 724, stores the images 204 of a CT scan in the CIPS storage 706. Further, at step 726, the gateway 704 sends the request, references to the stored images 204 of a CT scan, and other associated data via the HTTP Request Queue 708 to the DMQS 710. At step 728, the DMQS 710: (1) stores the study, series, scan and image metadata into a database via the DBMS 714, with correct associations; and (2) stores the images 204 of a CT scan in private cloud storage (not shown here) with the correct association to the study and series.

Example code snippets are provided as follows:

```
import { Series, Status, Study } from ′@annaliseai/api-
specifications′;
/ / multipart/form-data
/ / field=′data′
interface SeriesUploadMessage {
  study: Study;
  series: Series;
  images: Images [ ] ;
  attributesResult?: {
    isPrimary: boolean
  }
  error?: { code: string; message: string };
}
/ / field=′file′
/ / png blob with metadata
```

Step 726 example code snippets (gateway 704 to HTTP Request Queue 708) are provided as follows:

```
interface GatewayToRocketSeriesUploadMessage {
  studyInstanceUid: string;
  seriesInstanceUid: string;
  imageInstanceUids : string [ ] ;
  url: string; / /
}
```

Example code snippets (DMQS 710 to HTTP Request Queue 708) are provided as follows:

```
interface GrootToRocketSeriesRequest {
  studyInstanceUid: string;
  seriesInstanceUid: string;
  seriesVersionId: string;
}
interface GrootToRocketSeriesResponse {
  study InstanceUid: string;
  seriesInstanceUid: string;
  seriesVersionId: string;
  status: Status;
  url?: string / / full blob url
  error?: {
    code: string,
    message: string,
  }
  downsampled: {
    status: Status
    url?: string / / downsampled blob url
    error?: {
      code: string
      message: string
    }
  }
}
```

In an example, an archive contains 656 images in PNG format: 144 axial, 256 coronal and 256 sagittal images (based on the ontology tree in Table 1). Example code snippets between the CT connector of the integration layer 702 and HTTP Request Queue 708 are provided as follows:

```
enum ViewType {
  Axial,
  Coronal,
  Sagittal
}
interface CtbPredictionSegmentSliceInfo {
  index: number;
  isActivated: Boolean; / / true if slice activatedPixels > 0
  activatedPixels: number; / / pixel count in predicted
segmentation mask
}
interface CtbPredictionSegmentViews {
  [ViewType.AXIAL]: CtbPredictionSegmentSliceInfo[ ];
  [ViewType.CORONAL]: CtbPredictionSegmentSlice Info[ ];
  [ViewType.SAGITTAL]: CtbPredictionSegmentSliceInfo[ ];
}
interface CTBConnectorRocket {
  correlationId: string;
  organizationId: string;
  realm: string;
  studyInstanceUid: string;
  seriesInstanceUid: string;
  seriesVersionId: string;
  ctbAiPredictionId: string; / / generated uuid
  segmentationArchiveUrl: / / url to blob;
  segmentations: {
    segmentId: string / / generated uuid should match
CTBConnectorGroot
    label: string;
    views: CtbPredictionSegmentViews;
  } [ ]
}
```

FIG. 7C illustrates a method (process and data transfers) for processing and storage of medical imaging study results, according to an exemplary embodiment. This process is triggered by a ‘study complete’ event 730, which comprises a request sent from the integration layer 702 to notify the microservice that a particular study is finished with modality processing and has finalised image capturing for the study. This event will trigger the microservice to compile all related data required for the model to process images 204 of a CT scan and return a result with AI findings. The AI findings result will then be stored in the cloud storage 712.

In particular, at step 732 the gateway 704 forwards the study complete event to the DMQS 710. At step 734, the DMQS 710 sends the images 204 of a CT scan of the study to the MHS 716, via a reference to the associated images 204 of a CT scan in the cloud storage 712. At step 736 the MHS 716 fetches the images 204 of a CT scan from cloud storage 712, processes them along with associated metadata into protobufs, and forwards the data to the AIMS 718. The AIMS 718 then pre-processes and post-process the images 204 of a CT scan and sends them to the MLPS 720 at step 738.

Step 732 example code snippets (gateway 704 to DMQS 710) are provided as follows:

```
/ / multipart/form-data
/ / field=′data′
interface GatewayToGrootSeriesUploadMessage {
  study: Study;
  series: Series;
  images: Image[ ] ;
  attributesVersion: string;
  registrationVersion: string;
  attributesResult: {
    isPrimary: boolean
  }
  attributesError?: { code: string; message: string };
  registrationError?: { code: string; message: string };
}
/ / field=′file′
/ / png archive blob with metadata
```

0302 Example code snippets (DMQS 710 to integration layer 702 queue) are provided as follows:

```
type CTBClassificationLabel = string;
interface CTBConnectorIn {
  organizationId: string;
  realm: string
  study InstanceUid: string;
  seriesInstanceUid: string;
  seriesVersionId: string;
  ctbAiPredictionId: string; / / generated uuid
  productModelVersion: string;
  registrationModelVersion: string;
  attributesModelVersion: string;
  correlationId: string;
  registeredArchiveUrl: string; / / presigned url
}
```

Example code snippets (integration layer 702 to DMQS 710) are provided as follows:

Success Response

```
Typescript spec
enum Laterality {
  NONE,
  LEFT,
  RIGHT,
  BILATERAL
}
enum ViewType {
  AXIAL,
  CORONAL,
  SAGITTAL
}
enum CtbWindowingType {
  BRAIN,
  BONE,
  SOFT_TISSUE,
  STROKE,
  SUBDURAL,
}
type CTBClassificationLabel = string;
interface CtbPredictionSuccessResponse {
  correlationId: string;
  organizationId: string;
  realm: string;
  studyInstanceUid: string;
  seriesInstanceUid: string;
  seriesVersionId: string;
  ctbAiPredictionId: string; // generated uuid
  productModelVersion: string;
  classifications: {
    label: CTBClassificationLabel;
    predictionProbability: number;
    confidence: number;
    defaultWindow: CtbWindowingType;
    keyView: ViewType;
    keyViewSlices: Record<ViewType, number>;
  } [ ];
  lateralities: {
    label: string; / /
″intracranial_cranial_haemorrhage_laterality″
    relatedLabels: CTBClassificationLabel[ ]; // Mapping
between classification and laterality labels
    laterality: Laterality; / / array of 1: L/R/Bilat/None
  } [ ];
  segmentations: {
    label: string;
    relatedLabels: CTBClassificationLabel[ ]; / / Mapping
between classification and segmentation labels
    segmentId: string;
  } [ ];
}
JSON schema spec
{
    ″$schema″: ″http:/ /json-schema.org/draft-07/schema#″,
    ″definitions″: {
      ″CtbClassification″: {
        ″properties″: {
          ″confidence″: {
            ″type″: ″number″
          },
          ″keyView″: {
            ″$ref″: ″#/definitions/ViewType″
          } ,
          ″keyViewSlices″: {
            ″$ref″:
″#/definitions/Record<ViewType, number>″
          },
          ″label″: {
            ″type″: ″string″
          },
          ″predictionProbability″: {
            ″type″: ″number″
          },
          ″defaultWindow″: {
            ″$ref″: ″#/definitions/CtbWindowingType″
          }
        },
        ″required″: [
          ″confidence″,
          ″keyView″,
          ″keyViewSlices″,
          ″label″
          ″predictionProbability″,
          ″defaultWindow″
        ],
        ″type″: ″object″
      },
      ″CtbLaterality″: {
        ″properties″: {
          ″label″: {
            ″type″: ″string″
          } ,
          ″laterality″: {
            ″enum″: [
              ″BILATERAL″,
              ″LEFT″,
              ″NONE″,
              ″RIGHT″
            ],
            ″type″: ″string″
          },
          ″relatedLabels″: {
            ″items″: {
              ″type″: ″string″
            } ,
            ″type″: ″array″
          }
        },
        ″required″: [
          ″label″,
          ″laterality″,
          ″relatedLabels″
        ],
        ″type″: ″object″
      },
      ″CtbSegmentation″: {
        ″properties″: {
          ″label″: {
            ″type″: ″string″
          },
          ″relatedLabels″: {
            ″items″: {
              ″type″: ″string″
            },
            ″type″: ″array″
          },
          ″segmentId″: {
            ″type″: ″string″
          }
        },
        ″required″: [
          ″label″,
          ″relatedLabels″,
          ″segmentId″
        ],
        ″type″: ″object″
      },
      ″Record<ViewType, number>″: {
        ″description″: ″Construct a type with a set of
properties K of type T″,
        ″properties″: {
          ″AXIAL″: {
            ″type″: ″number″
          },
          ″CORONAL″: {
            ″type″: ″number″
          },
          ″SAGITTAL″: {
            ″type″: ″number″
          }
        } ,
        ″required″: [
          ″AXIAL″,
          ″CORONAL″,
          ″SAGITTAL″
        ],
        ″type″: ″object″
      },
      ″ViewType″: {
        ″enum″: [
          ″AXIAL″,
          ″CORONAL″,
          ″SAGITTAL″
        ],
        ″type″: ″string″
      },
      ″CtbWindowingType″: {
        ″enum″: [
          ″BRAIN″,
          ″BONE″,
          ″SOFT_TISSUE″,
          ″STROKE″,
          ″SUBDURAL ″
        ],
        ″type″: ″string″
      }
    } ,
    ″properties″: {
      ″classifications″: {
        ″items″: {
          ″$ref″: ″#/definitions/CtbClassification″
        },
        ″type″: ″array″
      },
      ″correlationId″: {
        ″type″: ″string″
      },
      ″ctbAiPredictionId″: {
        ″type″: ″string″
      },
      ″lateralities″: {
        ″items″: {
          ″$ref″: ″#/definitions/CtbLaterality″
        },
        ″type″: ″array″
      },
      ″organizationId″: {
        ″type″: ″string″
      },
      ″productModelVersion″: {
        ″type″: ″string″
      },
      ″realm″: {
        ″type″: ″string″
      },
      ″segmentations″: {
        ″items″: {
          ″$ref″: ″#/definitions/CtbSegmentation″
        },
        ″type″: ″array″
      },
      ″seriesInstanceUid″: {
        ″type″: ″string″
      },
      ″seriesVersionId″: {
        ″type″: ″string″
      },
      ″study InstanceUid″: {
        ″type″: ″string″
      }
    } ,
    ″required″: [
      ″classifications″,
      ″correlationId″,
      ″ctbAiPredictionId″,
      ″lateralities″,
      ″organizationId″,
      ″productModelVersion″,
      ″realm″,
      ″segmentations″,
      ″series InstanceUid″,
      ″seriesVersionId″,
      ″study InstanceUid″
    ],
    ″type″: ″object″
}
Error Response
interface CtbPredictionErrorResponse {
  correlationId: string;
  organizationId: string;
  realm: string;
  studyInstanceUid: string;
  seriesInstanceUid: string;
  seriesVersionId: string;
  ctbAiPredictionId: string;
  productModelVersion: string;
  error:
    code: PredictionErrorCode; / / TBD
    message: string;
  };
}
```

In exemplary embodiments, image pre-processing by the AIMS 718 may comprise one or more of the following steps:
1. transform the image 204 of a CT scan within the protobuf message received from the MHS 716 into a data structure accepted by the models executed by the MLPS 720 (e.g. TensorFlow tensor with datatype uint16 and input shape matching the deep learning models);
2. expand image dimensions to include a channels dimension (alongside height and width) if not existent;
3. convert the image 204 of a CT scan to grayscale if the channel dimension already exists and is not single channel;
4. convert the image datatype to a type supported by the models executed by the MLPS 720 (e.g. float32);
5. recalibrate the image pixels based on the linear model with the RescaleSlope and RescaleIntercept headers in DICOM image metadata;
6. shift pixel intensities such that the minimum value in each image 204 of a CT scan is 0;
7. ensure pixel intensity increases with black being 0 and white being the maximum data type—if the photometric interpretation is MONOCHROME1 (black=maximum data type value, white=0), reverse the pixel intensities by subtracting the values from one;
8. up/downsample and pad the image 204 of a CT scan with 0s to reshape it to the accepted model input shape if needed; and/or
9. rescale pixel intensities from [image minimum, 99.5^{th} percentile] to [0, 1] such that they represent a percentage of relative intensities within the image 204 of a CT scan (this allows the deep learning models to be trained to learn features based on relative values rather than absolute pixel intensities which can be prone to both systematic and random errors during image generation).

At step 740, the MLPS 720 executes one or more trained ML models (including the CNN model 200) to performs inference on the pre-processed images, producing predictions that are sent back to the AIMS 718 at step 742. The AIMS 718 processes the resulting findings, and transforms them to protobufs for transmission back to the MHS 716 at step 744. The MHS 716 transforms the received findings into JSON format, and returns them to the DQMS 710 at step 746, upon which they are stored to a database via the DBMS 714, ready for subsequent retrieval.

In exemplary embodiments, image post-processing by the AIMS 718 may comprise one or more of the following:
1. Preparing messages for HTTP Request Queue 708
2. Append CTB AI prediction ID
3. There shouldn't be any post-processing of segmentation results outside of the model
4. Whether classification and segmentation prediction confidence>threshold.
5. Segmentation and laterality results all subject to gating by classification as per normal (managed by model metadata)
   a. Mapping from classification labels to laterality labels are maintained
   b. Segmentation behaviour:
      i. many-to-one mapping between classification—each segmentation can be paired with multiple classification findings
      ii. if any of the related classification findings is detected, return the segmentation
      iii. Mapping for each segmentation to default direction (obtained from model)
         1. AIMS 718 returns the segmentation output for the direction specified by the relevant classification finding
      iv. Some findings are not mapped to any segmentation. For these, there is a default direction and index

A common problem when providing an automated medical analysis solution where the AI analysis is at least in part run remotely (such as e.g. on a cloud-based RIAS 110) is to improve the responsiveness perceived by the user (e.g. radiologist, radiographer or clinician) when receiving the results/predictions generated by the CNN model 200. This problem is particularly acute when the imaging modality is CT, where there are hundreds of images compared to the typical one to four images typically expected for chest x-rays.

This problem is addressed in example embodiments through features that can each be used independently, or in advantageous embodiments, synergistically.
1. Reduce the payload/data size that is transmitted from the RIAS 110 to the client of the workstation 112 via the network 102 (Internet).
2. Pre-fetch some or all images and (advantageously payload reduced) segmentation maps in the background and storing on a local cache in the client's workstation 112 to avoid wasting time that could be used to receive images and segmentation maps and therefore use the available Internet bandwidth from the moment user has opened a particular study. This is advantageous compared to downloading images and segmentation data on demand in response to user clicks because the user does not have to experience delay in waiting for the completion of the download.

Each of these features will now be described in greater detail.

In example embodiments, segmentation maps such as those described above, may be stored as PNG files. The segmentation maps can be transparent or transparent PNG files.

In the example where the medical scan images 204 are CT, the quantity of data which is often much larger than chest X-ray (CXR) images. This represents a very large amount of data to be sent to the user of the workstation 112 over the Internet 102, and may cause some delay in a user receiving the results of the deep learning analysis, and hence being able to use these to make a diagnosis in a timely manner. The problem is exacerbated if the user is located in an environment that has poor Internet connectivity or low Internet speeds, which is the case for a significant proportion of the world's population who may reside outside of urban areas.

In example embodiments, the image/pixel data is separated from the segmentation data (metadata). The segmentation data identifies where in the image a particular radiological finding is located, and may be presented to the user with a coloured outline with semi-transparent coloured shading. The viewer component 701 is then able to display the image and the segmentation map as two images on top of each other (e.g. a segment image overlying the image 204 of a CT scan).

This step has a very significant impact on improving the user experience and increasing UI responsiveness, because a smaller data size is required to be transmitted to communicate the same information without any reduction in quality of the information being communicated.

Segmentation maps can be stored as PNG files, in particular transparent PNG files. As mentioned above, PNG is advantageous because it supports lossless data compression, and transparency. Additionally, PNG is a widely supported file format. Other file formats may be used such as JPEG, which has wide support but does not support transparency or lossless compression, or GIF, which supports transparency but not lossless compression and is not as widely supported as PNG or JPEG.

In some embodiments, instead of transparent PNGs, the segmentation maps could be stored as area maps (also called bounding boxes). Area maps may advantageously reduce file size because only the corners need to be defined. This may be advantageously used when only a region of a CTB slice image 204 has to be highlighted, not a particular shape of the region. This may not be adequate or advantageous for all situations. Further, the use of area maps may create extra steps on the server-side, as area maps have to be obtained from the segmentation information (array of 0's and 1's) received from the deep learning model(s).

Alternatively, in other embodiments the segmentation maps may be stored as SVG files. SVG is a vector based image format. This advantageously enables the interactive viewer component 701 to have more control over the display of the information. In particular, vector images are scalable (they can be scaled to any dimension without quality loss, and are as such resolution independent), and support the addition of animations and other types of editing. Further, vector based image formats may be able to store the information in smaller files than bitmap formats (such as PNG or JPEG) as their scalability enables saving the image 204 of a CT scan at a minimal file size.

Another example embodiment may provide a pre-fetching module is which is configured to pre-fetch the images 204 of a CT scan and segmentation maps. The feature is also referred to as lazy loading because the user is not required to do anything for the data to transmit passively in the background. In some embodiments, pre-fetching may occur without user knowledge or there may be a visual element displayed in the user interface such as a status bar that may indicate download activity or download progress. Therefore, the interaction by the user with the viewer component 701 ultimately is not perceived as laggy to the user because all the necessary data is stored in the local cache in the client's workstation 112 ahead of the time it is required to be presented to the user of the workstation 112. The need to download data in real-time is obviated and avoids the user of the workstation 112 having to wait or see a screen flicker because data needs to be downloaded at that moment for processing and presentation to the user, e.g. in the viewer component 701.

Advantageously, in a further embodiment, the pre-fetching of the images 204 of a CT scan and segmentation maps is performed intelligently, by creating a transmission queue that includes logic that predicts the next likely radiological findings that will draw the attention of the user. For example, important (or clinically significant/high priority) radiological findings and their segmentation maps are ordered at the start of the transmission queue and retrieved first, and the less important ones following. Alternatively or additionally, the system may detect the position of the mouse cursor within the interactive viewer component 701 on a specific radiological finding (active position), and retrieve images/segmentation maps corresponding to the adjacent radiological findings (previous and next), first. The priority logic is configured to progressively expand the retrieval of images/segmentation maps from further previous and further next which is ordered correspondingly in the transmission queue. The transmission queue is re-adjustable depending on a change in the mouse cursor position to determine what the active position is and the specific radiological finding.

The code snippets below represent exemplary implementations of these functions.

```
//Pre-fetching CTB images:
return (data?.images ?? [ ]).reduce((acc: UlImageUrl, image) => {
  const url = image.targets?.jpeg?.url;
  // Pre-fetch image to avoid Ul flickering when displaying it the
  // first time
  new Image( ).src = url;
  return {
    ... acc,
    [image.imageInstanceUid]: url,
  };
}, { });
//Pre-fetching segmentation maps:
return findingsSegment.segments.reduce((acc: UISegmentUrl, segment) => {
  // Pre-fetch image to avoid Ul flickering when displaying it the
  // first time
  new Image( ).src = segment.url;
  return {
    ...acc,
    [segment.id: segment.url,
    };
  }, { });
```

The functions depict a loop through image URLs that a CIPS 706 passes to the interactive viewer component 701 for any given study.

The pre-fetching module enables the interactive viewer component 701 to be at least one step ahead of the user's attention or intended action, therefore it is perceived by the user to be seamless and snappy.

The functionalities described above can be implemented as part of the CIPS 706 that stores study images, AI segment results and handles image conversions and manipulations. A service gateway is configured to trigger events to CIPS 706 for image uploads and processing. CIPS 706 is responsible for receiving, converting and storing images into secure cloud storage 712. CIPS 706 is configured: (a) to provide image processing capabilities; (b) to provide both an asynchronous and synchronous image storage and retrieval mechanisms; and (c) to store model segmentation findings (generated by the CNN model 200).

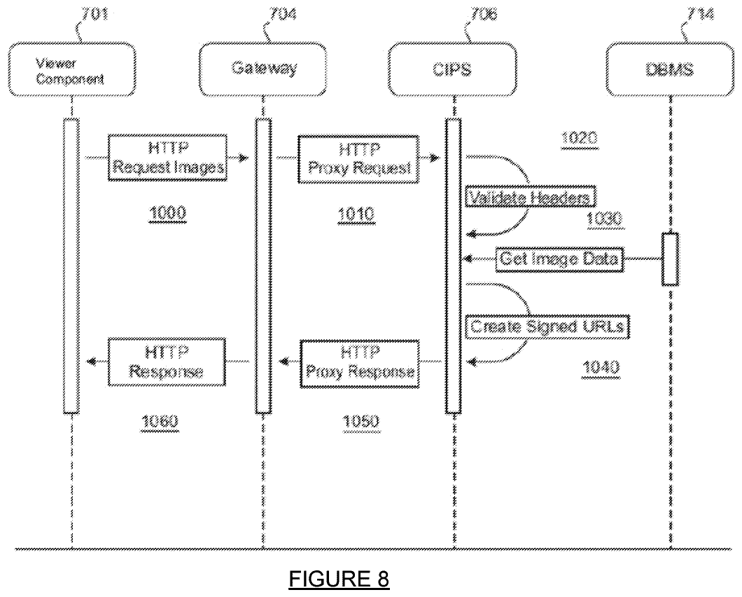

Referring to FIG. 8, a method of providing image data to the viewer component 701 will now be described. At step 1000 the viewer component 701 (client) sends image instance UIDs to the service gateway (Receiver) using the HTTP protocol. At step 1110, the gateway (client) forwards the request with payload to CIPS (Receiver). CIPS 706 optionally validates the header of the request at step 1020, and then retrieves (step 1030) the image data from the DBMS. CIPS 706 then generates 1040 a secure cloud storage image URL for the image, which the viewer component 701 can use to fetch and display images. CIPS 706 responds to the request with the image data via the gateway, which then forwards this to the viewer component 701 at steps 1050, 1060, using the HTTP protocol.

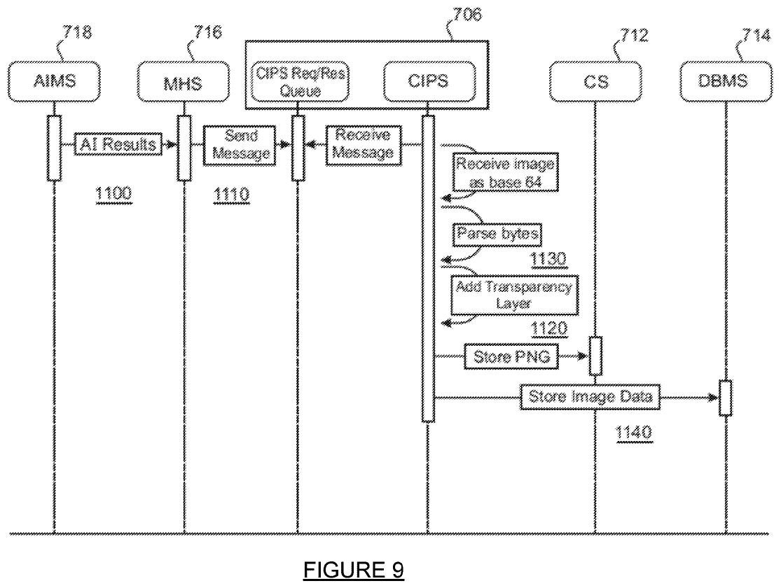

Referring to FIG. 9, a method of processing a segmentation image result will now be described. At step 1100, the AI Model Service (AIMS, client) sends AI findings results including a segmentation image and metadata to the Model Handler Service (MHS, Receiver). At step 1110, MHS sends the segmentation image results as a PNG to CIPS 706. At step 1120, CIPS 706 stores the segmentation image as a PNG in secure cloud storage. At step 1130, CIPS 706 manipulates the image 204 of a CT scan (or a generated slice from the spatial 3D tensor) by adding a layer of transparent pixels on top of black pixels. At step 1140, CIPS 706 stores the segmentation image metadata, the image secure URL location and the study finding metadata to the DBMS.

The CNN model 200 can be configured to generate, using the series of anatomical images 204 by a preprocessing layer: a spatial 3D tensor which represents a 3D spatial model of the head of the subject. The CNN model 200 can be configured to generate, using the spatial 3D tensor: one or more 3D segmentation masks, each 3D segmentation mask representing a localization in 3D space of a respective visual anomaly finding classified as being present by the CNN model 200. The CNN model 200 can be configured to generate, using each 3D segmentation mask: respective segmentation maps of that 3D segmentation mask for at least one anatomical plane.

The CNN model 200 can be configured to generate, for display on the viewer component 701, an overlay of a first segmentation map of the segmentation maps of a first respective visual anomaly finding in one of the anatomical planes onto an anatomical slice of the CT scan in the one of the anatomical planes.

The CNN model 200 can be configured to generate, selecting the at least one anatomical plane to be displayed as a default based on a pair of the respective visual anomaly finding and the respective anatomical plane as indicated in Table 1.

In an example, the generating the display can include selecting the one of the anatomical planes to display the anatomical slice in dependence of the first respective visual anomaly finding, wherein the one of the anatomical planes is: sagittal, coronal, or transverse.

In an example, the generating for display can include selectively adding or selectively removing the first segmentation map.

In an example, the generating for display includes overlaying a second segmentation map of the segmentation maps of a second respective visual anomaly finding onto the anatomical slice and simultaneously displayed with the first segmentation map.

In an example, the generating for display includes the first segmentation map including first non-transparent pixels with a first level of transparency corresponding to a first area of the respective anatomical image where the first visual anomaly finding is classified by the CNN model as being present, and the second segmentation map including second non-transparent pixels with a second level of transparency corresponding to a second area of the respective anatomical image where the second visual anomaly finding is classified by the CNN model as being present.

In an example, the generating for display includes the anatomical slice being generated for display in a default windowing type in dependence of the first respective visual anomaly finding, wherein the default windowing type is soft tissue, bone, stroke, subdural, or brain.

In an example, the default view for the first visual anomaly finding are any one pair in Table 1 of respective default windowing types for respective visual anomaly findings.

In an example, the respective default view for each respective visual anomaly finding for the generating for display are all listed in the Table 1.

In another example, the generating for display includes the CNN model 200 generating or selecting the anatomical slice to be displayed as a default slice or key slice in dependence of an attention weight 3D tensor generated by the CNN model 200.

In another example, the generating for display includes the CNN model 200 generating or selecting the anatomical slice to be displayed as a default slice or key slice which is associated with the first segmentation map having a highest area covered of all of the segmentation maps.

In an example, the generating for display includes generating for display a 3D spatial visualization of a first segmentation 3D tensor of the segmentation 3D tensors of a first respective visual anomaly finding simultaneously with the 3D spatial model.

In an example, the generating for display includes a second segmentation 3D tensor of the segmentation 3D tensors of a second respective visual anomaly finding simultaneously with the 3D spatial model and simultaneously displayed with the first segmentation 3D tensor.

In an example, the generating for display includes selectively adding or removing the first segmentation 3D tensor.

In an example, the generating for display includes a left-right laterality at least one visual anomaly finding classified as being present by the CNN model 200. In an example, the left-right laterality is generated for each of the respective visual anomaly findings with laterality as indicated in Table 1 and classified as being present.

In an example, the generating for display includes generating for display at least two of the possible visual anomaly findings classified as being present by the CNN model 200 in a hierarchal relationship based on the hierarchical ontology tree.

As illustrated with reference to FIG. 10, the system 100 may have CXR and CTB capability. The following options are envisaged when running both CXR and CTB workflows: i) Single instance of integration layer 702 (integration adapter) with single group of DICOM receiver; ii) Single instance of Integration Adapter with difference groups of DICOM receiver for CXR and CTB; and iii) Two instances of integration layers (702/702A as shown in FIG. 10).

The predicted findings are displayed to the user in the viewer component 701 using the key slice selection criteria described above.

There are different options to identify the DICOM data and map to pipelines, including: i) Service-Object Pair (SOP) class (image level, required); ii) Modality (Series level, required); or iii) Modality (Series level, required) and Body Part (Series level, optional).

The DICOM transmitter includes the following functions: Consume dataset from queue; Parse as vision request (headers and pixel data); Parse as database records (headers only); Send vision request; and Save records in database.

The CTB DICOM transmitter includes the following functions: Consume dataset from queue; Parse as database records (headers only); Save records in database; and Save DICOM images in the MinlO file system.

In an example, a CT decision support tool includes the CNN model 200 substantially as described above, has been evaluated for its ability to assist clinicians in the interpretation of NCCTB scans. The study had two endpoints: (1) How does the classification performance of radiologists change when the deep learning model is used as a decision support adjunct? (2) How does a comprehensive deep learning model perform and how does its performance compare to that of practising radiologists?

The CT decision support tool includes a hierarchical menu which displays at least some of the possible visual anomaly findings in the hierarchal relationship from the hierarchical ontology tree. Through the tool, labelling of a first possible visual anomaly label at a first hierarchal level of the hierarchical ontology tree as being present automatically labels a second possible visual anomaly label at the second hierarchal level of the hierarchical ontology tree as being present.

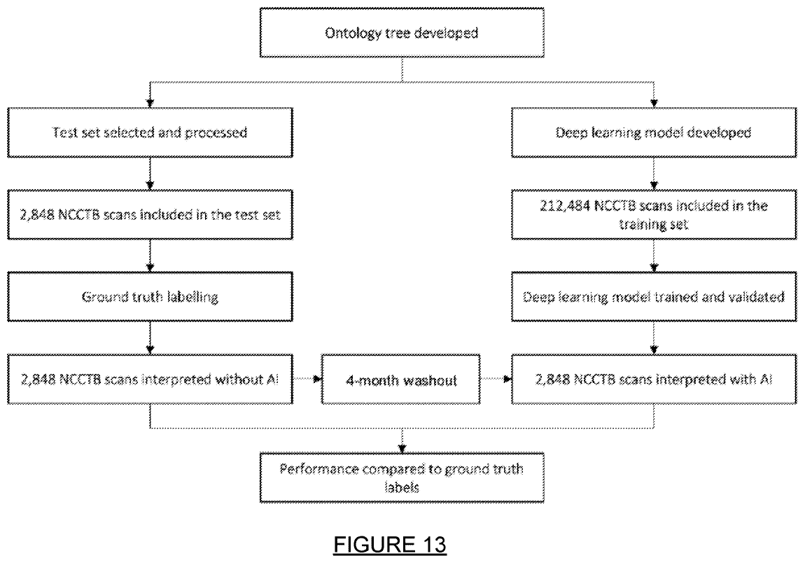

FIG. 13 illustrates a multi-reader multi-case (MRMC) study evaluated the detection accuracy of 32 radiologists with and without the aid of the CNN model 200.

Radiologists first interpreted cases without access to the deep learning tool, then re-interpreted the same set of cases with assistance from the CNN model 200 following a four-month (124 day) wash-out period.

The model performance of the CNN model 200 can be at least partially attributed to the large number of studies labelled by radiologists for model training using a prospectively defined ontology. Table 2 illustrates the study dataset details. Data are listed as n (%), mean (SD) or median (IQR).

Model development and evaluation involved three groups of radiologists performing distinct functions: (1) labelling of the training dataset (157 consultant radiologists from Vietnam), (2) ground truth labelling of the test dataset (three specialist neuroradiologists from Australia), and (3) interpretation of the test dataset in the MRMC study (32 consultant radiologists from Vietnam). Labelling of the training dataset identified the radiological findings present on each case, as defined by an ontology tree prospectively developed by consultant neuroradiologists that contained 214 clinical findings (192 child findings and 22 parents). Note that the CNN model 200 can consider, e.g. 351 classifications, as the CNN model 200 can deal with hidden stratifications among other things, with a subset of such classifications being clinically relevant. Ground truth labelling identified the radiological findings present in the test dataset used in this MRMC study. A total of 192 fully accredited radiologists from Australia and Vietnam took part in these processes.

An overview of the study design is presented in FIG. 13. As illustrated in FIG. 13, an ontology tree was developed, and clinical findings were labelled by radiologists. The test set contained past and future images and reports, which facilitated ground truth labelling by three subspecialist neuroradiologists. The CNN model 200 was trained with five-fold cross-validation. The test set was assessed by 32 radiologists with and without model assistance.

A NCCTB clinical finding ontology tree was developed, specifying clinically relevant findings and describing relationships between these findings. Each of the 214 findings was defined by a consensus of four Australian radiologists, including three subspecialist neuroradiologists. Radiologists engaged in labelling and evaluation were trained to identify NCCTB findings according to these definitions. Clinically similar findings were grouped together as ‘children’ under an umbrella ‘parent’ label.

The 212,484 NCCTBs in the training dataset were drawn from a large private radiology group in Australia and included scans from inpatient, outpatient, and emergency settings. Inclusion criteria for the MRMC test dataset were age >18 years, and series slice thickness less than 1.5 mm. All patient data were de-identified. Patient IDs and case IDs were anonymised while retaining the temporal and logical association between cases. Images were preserved at the original resolution and bit-depth.

All NCCTBs were independently labelled by three to eight radiologists selected from a pool of 157. Cases were randomised and placed in a queue. As radiologists completed each case, they were allocated the next incomplete case that they had not yet labelled according to queue order. Each case was labelled by at least three different radiologists. Each radiologist was given the same data for each case but was blinded to labels generated by the other radiologists. The radiology report, age and sex were provided, along with all series in the study (including contrast scans), and paired CT or magnetic resonance imaging (MRI) scans close in time to the NCCTB scan of interest (within 14 days).

Radiologists were trained prior to labelling. This involved familiarization with the annotation tool, reviewing the definitions of each finding within the ontology tree, and training on a separate curated dataset of 183 NCCTBs covering most clinical findings within the tree. The performance of each labeller was assessed with the F1 metric. Each training data labeller required an F1 score exceeding 0.50. Ongoing training and feedback was provided to ensure labelling was well-aligned to the definition of each label.

Labels included classification labels on a case level, indicating whether each finding was “present” versus “absent” in the case, as well as 3D segmentation for relevant findings. The consensus for each finding in each case was generated as a score between 0 and 1 using the Dawid-Skene consensus algorithm, which accounts for the relative accuracies of each labeller for each finding. Segmentation maps were labelled by a single radiologist to visually localize the pathology and were used to train the CNN model to produce overlay outputs.

In addition to training the CNN model 200 on the original labels, derived training labels were generated based on the ontology tree. Parent findings were automatically labelled based on child labels. As such, the CNN model 200 learnt from the original labels and from the structure of the ontology tree.

For example, the CNN model 200 is trained through a labelling tool for a plurality of sample CT scans that allows at least one expert to select labels presented in a hierarchical menu which displays at least some of the possible visual anomaly findings in the hierarchal relationship from the hierarchical ontology tree. For example, labelling of a first possible visual anomaly label at the first hierarchal level of the hierarchical ontology tree as being present automatically labels a second possible visual anomaly label at the second hierarchal level of the hierarchical ontology tree as being present.

The CNN model 200 was penalised less if the CNN model 200 classified the original label incorrectly but still correctly classified the parent. Any particular images that were improperly classified by the CNN model 200 but now corrected according to the ontology tree can be re-inserted into the CNN model 200 with applicable labels for retraining. In an example, the ontology tree module 382 (FIG. 3F) uses the ontology tree to update any of the classification predictions of parent labels that were classified as being absent that were inconsistent with the respective child labels, and updating those parent labels as being present.

A power analysis determined that a minimum MRMC test dataset of 2,848 cases was required to detect a mean difference in area under the receiver operating characteristic curve (AUC) of 0.02 in the detection accuracy of 30 radiologists labelling all findings (alpha=0.05, beta=0.8). Cases were drawn from labelled data to achieve a sufficient number of cases per finding, while keeping the total number of cases as low as possible. MRMC test dataset cases were excluded from model training at the patient level.

Case selection was controlled for the co-occurrence of any two findings as follows. If their co-occurrence in the wider dataset was less than 50%, their co-occurrence would not exceed 50% in the test dataset. If their co-occurrence in the wider dataset was 50% or greater, their co-occurrence in the test dataset did not exceed more than 75%.

Ground truth labels for the MRMC test dataset were determined by one of three fully-credentialled, fellowship-trained subspecialist neuroradiologists reviewing the Dawid-Skene consensus of the radiologist labels. Difficult cases were resolved by consensus discussion between the three neuroradiologists. These neuroradiologists had access to anonymised clinical information, past and future radiological investigations, including MRI, and radiology reports. They did not have access to the outputs of the CNN model.

The deep learning model (e.g. CNN model 200) includes an ensemble of five CNNs trained using five-fold cross-validation. The model had three heads: one for classification, one for left-right localization (laterality), and one for segmentation. Models were based on the ResNet, Y-Net and ViT architectures. Class imbalance was mitigated using class-balanced loss weighting and super-sampling of instances with segmentation labels. Study endpoints addressed the performance of the classification model (v1.0). Segmentation was not directly evaluated, although segmentation output was displayed to MRMC participants.

The 144 findings selected for inclusion in the viewer component 701 were determined based on clinical and statistical considerations. Included findings were required to (1) achieve an AUC of at least 0.80; (2) be able to achieve a precision of 0.20 at any threshold; (3) be trained on a total of at least 50 positive cases and at least 20 cases in the test set; and (4) demonstrate performance that was not lower than previously published AI performance for comparable clinical findings. F-beta values were chosen by the team of subspecialist neuroradiologists, based on the criticality of the finding. The higher the criticality, the less tolerance for missing a finding and thus a higher F-beta was chosen to improve the sensitivity of the model.

Thirty-two radiologists, each with 2-21 years of clinical experience after completing radiology specialist training (median=8 years), each interpreted all cases in the MRMC dataset. Patient age and sex were shown but no radiological report or other comparison images were provided. Radiologists were asked to rate their confidence in the presence of each of the 214 findings in the ontology tree (192 children and 22 parents) using a seven-point scale. Labelling, ground truth annotation, and interpretation were performed using the same custom-built, web-based DICOM viewer component 701 used for the development of the training dataset. When using the DICOM viewer component 701, radiologists were able to scroll through the study and select clinical findings in a list. The logical relationship between parent and child findings was enforced, such that a parent finding could not be selected without selecting a child, and vice versa. The viewer component 701 listed findings detected by the CNN model. When a finding was selected by the user, the viewer component 701 switched to the most appropriate viewing window and slice for that finding. For a subset of the findings, a segmentation overlay was displayed. Radiologist interaction was performed on diagnostic-quality monitors and hardware. Interpretation times were recorded by the DICOM viewer component 701.

Thirty-two radiologists were trained on ontology tree definitions and the labelling methodology using the DICOM viewer component 701. They then each independently evaluated all 2,848 studies without model assistance. After a wash-out period, training on use of the viewer component 701 was provided again, and the same radiologists independently evaluated the studies again with model assistance. Studies were newly randomised and presented in the same order for all radiologists. Radiologists did not have access to their first arm labelling results during the second arm of the project.

The primary objective of the study involved measuring the difference in radiologist detection performance with and without assistance from the CNN model 200. The secondary objective involved comparing the performance metrics of unassisted readers with the standalone CNN model 200.

For the primary objective, the differences in AUC, Matthews Correlation Coefficient (MCC), positive predictive value (PPV), sensitivity, and specificity for each finding were calculated to assess performance. The MCC represents the quality of a binary classifier, ranging from −1 (total disagreement) to +1 (total agreement). Receiver operating characteristic (ROC) curves were plotted. FDA iMRMC v4.0.3 software and the generalized Roe and Metz model were used to analyze radiologist performance (AUCs) with and without assistance from the CNN model 200. An AUC difference greater than 0.05 and an MCC difference greater than 0.1 were considered clinically significant. Confidence scores ≥1 on the seven-point rating scale were considered positive when calculating binarized performance metrics. Bootstrapping was used to determine if there was a statistically significant difference in average radiologist MCC for each finding between arms. Where bootstrapping was performed, 10,000 bootstraps of all cases were drawn, with resampling, to estimate the empirical distribution of the parameter concerned. For the secondary objective, the AUC of the CNN model was compared to the average unassisted radiologist AUC for each finding using a bootstrapping technique. The Benjamini-Hochberg procedure (Benjamini Y., Hochberg Y., ‘Controlling the false discovery rate: a practical and powerful approach to multiple testing’, J R Stat Soc Ser B 1995, 57: 289-300, incorporated herein by reference in its entirety) was used to control the false discovery rate, accounting for multiple comparisons. Analyses were conducted using Python 3.8.0. Pandas 1.2.4, SciPy 1.7.1, Matplotlib 3.4.3, Seaborn 0.11.2 and NumPy 1.20 were used for data processing, visualization, and significance testing. Scikit-learn 0.24.2 was used for the CNN model design, training and validation. MRIcroGL 1.2 was used for 3D visualisation of clinical findings. Three researchers independently conducted analyses to verify results. The statistical methodology and analysis were verified by an independent professor of biostatistics at Melbourne University.

An example of the CNN model 200 was used to comprehensively classify 144 clinical findings on NCCTB scans and tested its effects on radiologist interpretation in an experimental setting by conducting a multi-reader, multi-case (MRMC) study. A total of 212,484 scans were labelled by practicing radiologists and comprised the training dataset. The median number of training cases per clinical finding was 7 (IQR: 4-10). A total of 2,848 NCCTB scans were included in the MRMC test dataset (Table 2). They were interpreted by 32 radiologists with and without access to the model. A five-month washout period was imposed between study arms. One hundred and twenty findings passed performance evaluation and were selected for inclusion in the CNN model.

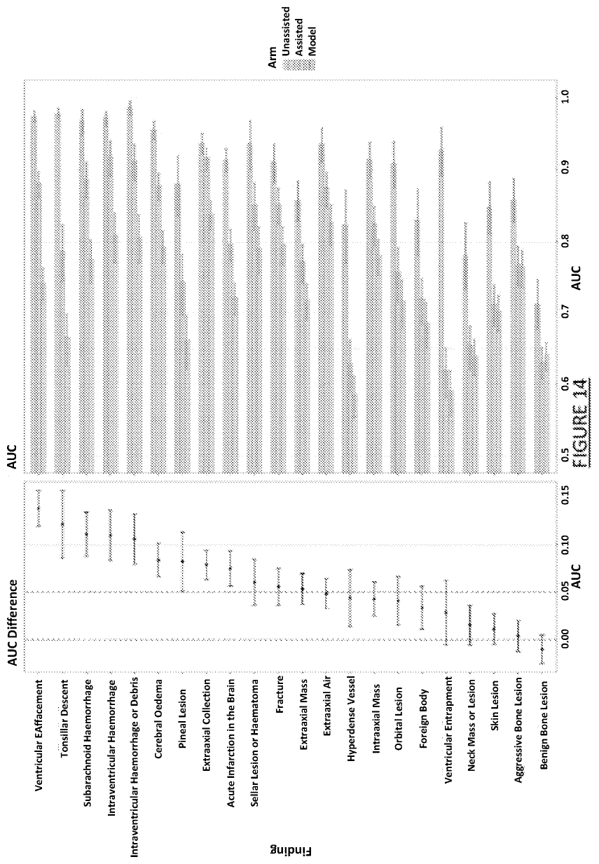

Model assistance improved radiologist interpretation performance. Unassisted and assisted radiologists demonstrated an average AUC of 0.73 and 0.79 across the 22 parent findings, respectively. Three child findings had too few cases for the iMRMC software to calculate reader performance (“enlarged vestibular aqueduct”: 0, “intracranial pressure monitor”: 0, and “longus colli calcification”: 1). Unassisted radiologists demonstrated an average AUC of 0.68 across the remaining 189 child findings. The lowest AUC was obtained for “intraventricular debris” (0.50, 95% CI 0.49-0.51). The highest AUCs were obtained for “deep brain stimulation (DBS) electrodes” (0.97, 95% CI 0.95-0.99), “ventriculoperitoneal (VP) shunt” (0.96, 95% CI 0.95-0.97) and “aneurysm coils” (0.95, 95% CI 0.93-0.98). Assisted radiologists demonstrated an average AUC of 0.72 across the 189 child findings. The lowest AUC was obtained for “intraventricular debris” (0.50, 95% CI 0.50-0.50). The highest AUCs were obtained for “DBS electrodes” (0.99, 95% CI 0.99-1.00), “aneurysm coils” (0.97, 95% CI 0.94-0.99) and “VP shunt” (0.97, 95% CI 0.95-0.96). Change in radiologist AUC when assisted by the model was positive and significant for 92 of the 189 child findings (49%). The three findings that demonstrated the largest AUC increase were “uncal herniation” (AUC increase 0.19, 95% CI 0.14-0.24), “sulcal effacement” (AUC increase 0.19, 95% CI 0.16-0.21), and “tonsillar herniation” (AUC increase 0.19, 95% CI 0.12-0.25). One hundred and fifty-eight findings (84%) were clinically non-inferior. Seventeen AUC decrements were identified when the model 200 was used as an assistant for the radiologists. One finding was clinically inferior (“cerebellar agenesis”) and sixteen were statistically inferior (“cavum septum pellucidum”, “aggressive mixed lesion calvarial”, “non aggressive bone lesion”, “extradural haematoma”, “diffuse axonal injury”, “skin abscess”, “atlanto-axial subluxation”, “brainstem atrophy”, “fracture C1-2”, “ventricular mass”, “skull vault haemangioma”, “lytic spine lesion”, “striatocapsular slit-like chronic haemorrhage”, “intraaxial lesion CSF cyst”, “orbital abscess”, and “ossicular chain disruption”). FIG. 14 illustrates assisted and unassisted radiologist AUCs for the 22 parent findings. Model use was associated with a significantly lower mean interpretation time (26 seconds faster with model assistance, 95% CI 13-41 seconds).

FIG. 14 illustrates the change in AUC of parent findings when radiologists were aided by the deep learning model. Mean AUCs of the model, unassisted radiologists and assisted radiologists and change in AUC, along with adjusted 95% CIs are shown for each parent finding.

Eighty-one child findings demonstrated a statistically significant improvement in MCC when radiologists used the CNN model 200 as an assistant for the radiologists. One hundred and sixty-nine child findings were clinically non-inferior. Nineteen findings were inconclusive as the lower bounds of the 95% confidence interval were less than −0.1 and the upper bounds were greater than zero.

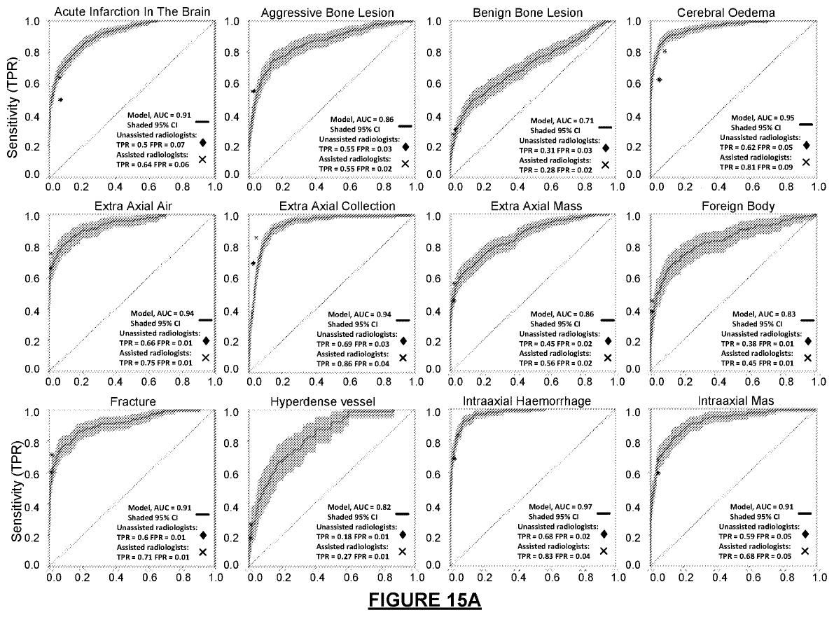

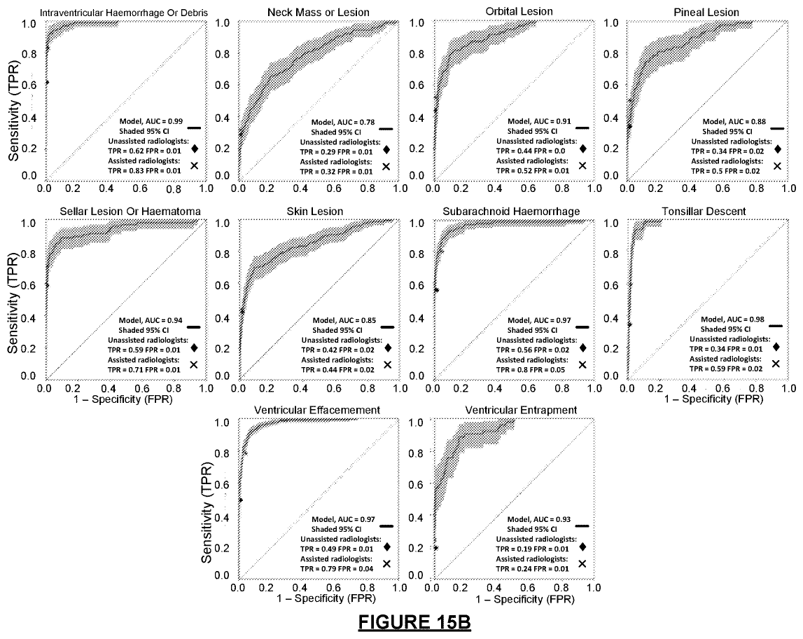

The model alone demonstrated an average AUC of 0.930 across all 144 model findings and 0.90 across the parent findings. The lowest AUCs were obtained for “non-aggressive extra-axial fat density” (0.747, 95% CI14 0.637-0.850) and “non-aggressive bone lesion” (0.755, 95% CI 0.698-0.809). The highest AUCs of 1.000 were obtained for “DBS electrodes (95% CI 1.000-1.000), and “cochlear implant” (95% CI 1.000-1.000). Model AUC was statistically superior to unassisted radiologist performance for 142 clinical findings. The two remaining findings were inconclusive, as the lower bounds of the AUC change lay below −0.05 but the upper bounds lay above zero. ROC curves comparing the performance of the model with the mean performance of radiologists are presented in FIG. 15 (parent findings).

FIG. 15 illustrates ROC curves for the parent findings demonstrating the performance of the model, and the mean performance of the assisted and unassisted radiologists.

FIG. 16 illustrates the effect of the CNN model 200 on the recall and precision of radiologists for all findings, averaged within four groups based on the F-beta values chosen for each finding. An F-beta was chosen for each finding by the neuroradiologists based on the clinical importance of the finding, the higher the clinical importance, the higher the value of F-beta was chosen. Increasing the F-beta value reduced the threshold of the model for that finding, increasing both the number of true positives and false positives. Improving sensitivity comes at the cost of reducing precision, triggering more false positives. FIG. 16 indicates that the precision dominant reporting of findings by the unaided radiologist can be swayed towards a recall dominance by increasing the F-beta of the model. Even with an F-beta of 1, radiologists became more sensitive without losing precision.

FIG. 16 illustrates the precision and recall for the unassisted and AI aided (with the CNN model 200) radiologists for every finding, averaged within 4 groups based on the chosen F beta levels for each finding. The arrows indicate the shift in recall and precision of the radiologists when aided by the CNN model 200.

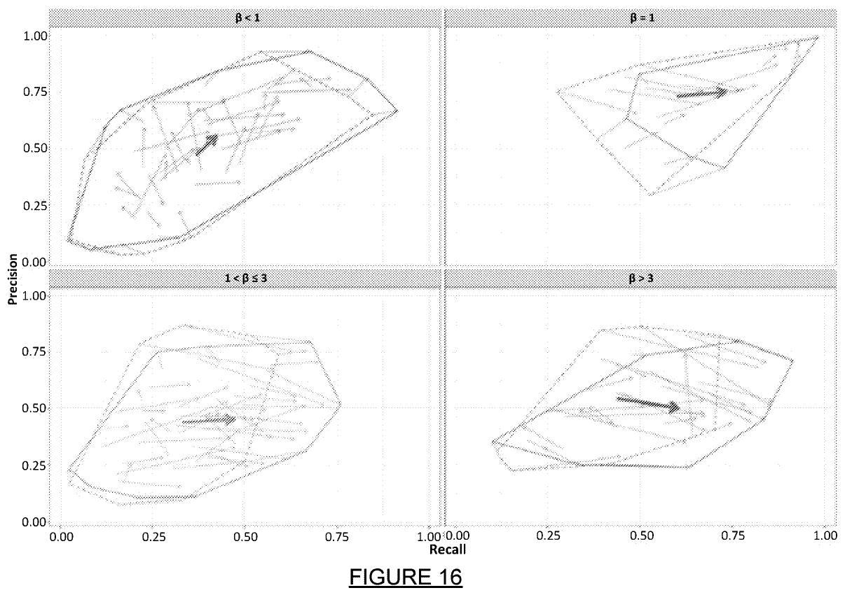

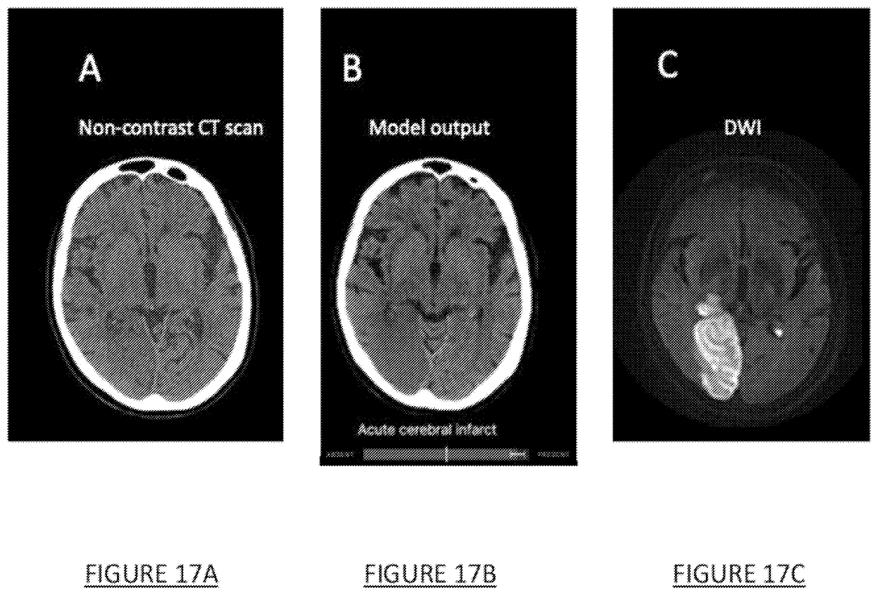

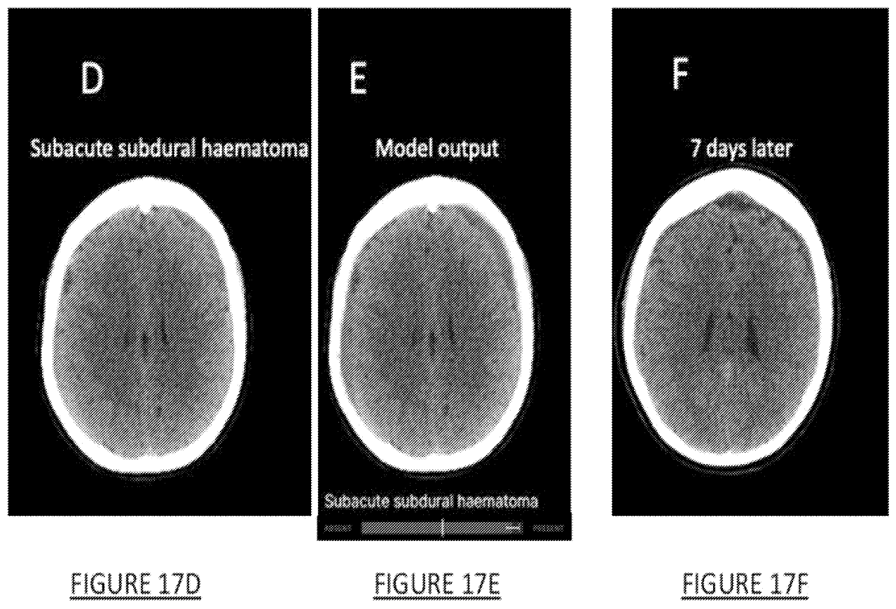

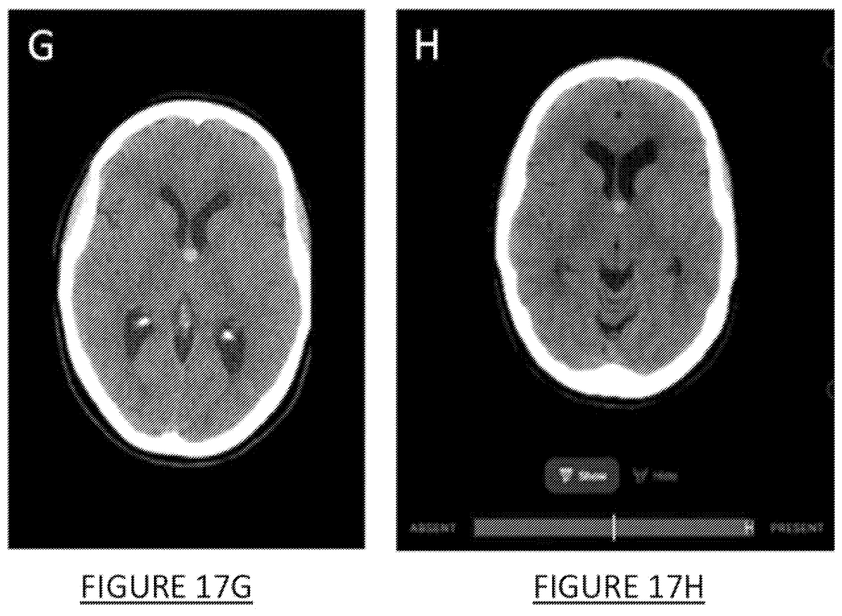

FIGS. 17A to 17H illustrate cases of acute cerebral infarction with subtle changes on the NCCTB study that were missed by most of the unassisted radiologists but were identified by most radiologists when using the CNN model. FIG. 17B and FIG. 17E show a case of colloid cyst causing obstructive hydrocephalus.

Specifically FIG. 17A illustrates non-contrast CT brain study of a 79-year-old female who presented with acute stroke symptoms. Subtle hypodensity in the right occipital lobe was missed by 30 of the 32 readers in the first arm of the study (unaugmented), but detected by 26 readers in the 2nd arm when using the model 200 as an assistant for the radiologists. FIG. 17B illustrates output of the CNN model. The model accurately localized the large area of infarction within the right occipital lobe (purple shading). Note the high level of confidence of the model, indicated by the bar at the bottom of the image. The bar to the right of the image indicates the brain slices that contain acute infarction. FIG. 17C illustrates a DWI image clearly showing the area of acute infarction in this patient. FIG. 17D illustrates an example of a small bilateral isodense subacute subdural haematomas. FIG. 17E illustrates that the haematomas were characterised by the CNN model as subacute subdural haematomas and localized with purple shading. FIG. 17F illustrates a CT scan performed 7 days later. The haematoma is more conspicuous on the later scan as it evolves to become hypodense. FIG. 17G illustrates a non-contrast CT brain study of a 56-year-old female who presented with severe headache. FIG. 17H illustrates a small hyperdense lesion in the roof of the third ventricle consistent with a colloid cyst as outlined by the CNN model 200. The CNN model 200 also picks the mild hydrocephalus associated with the mass.

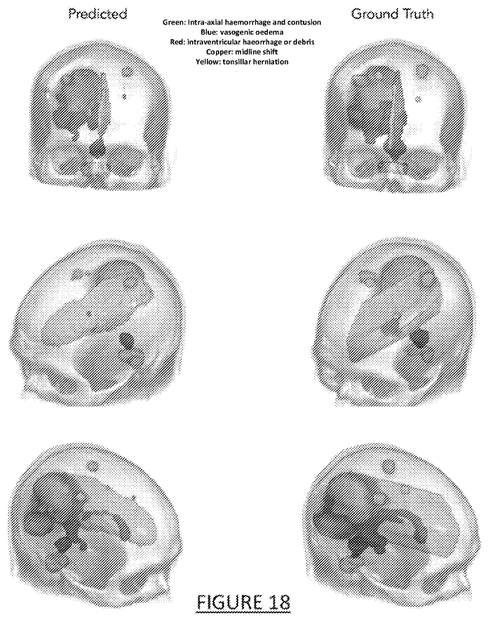

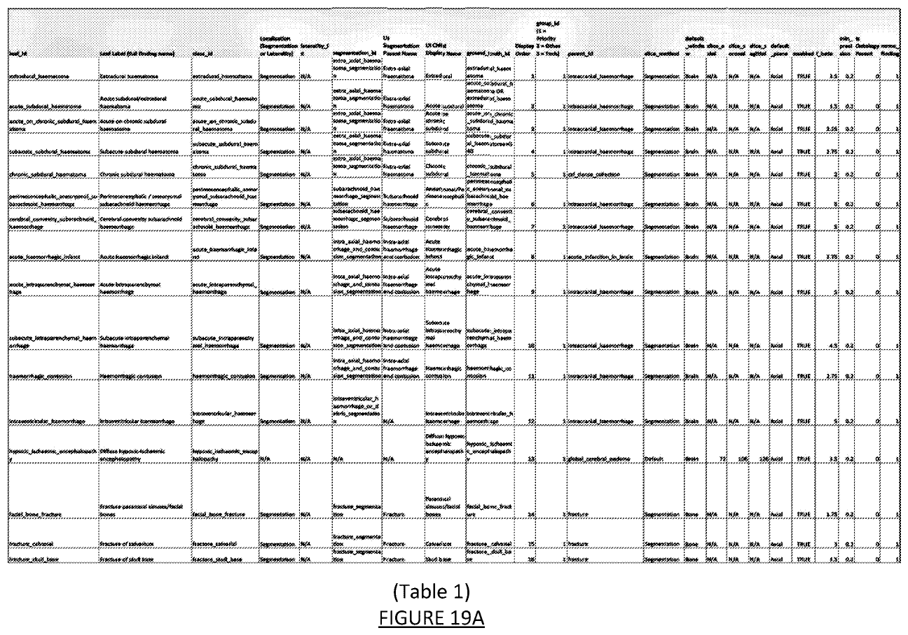

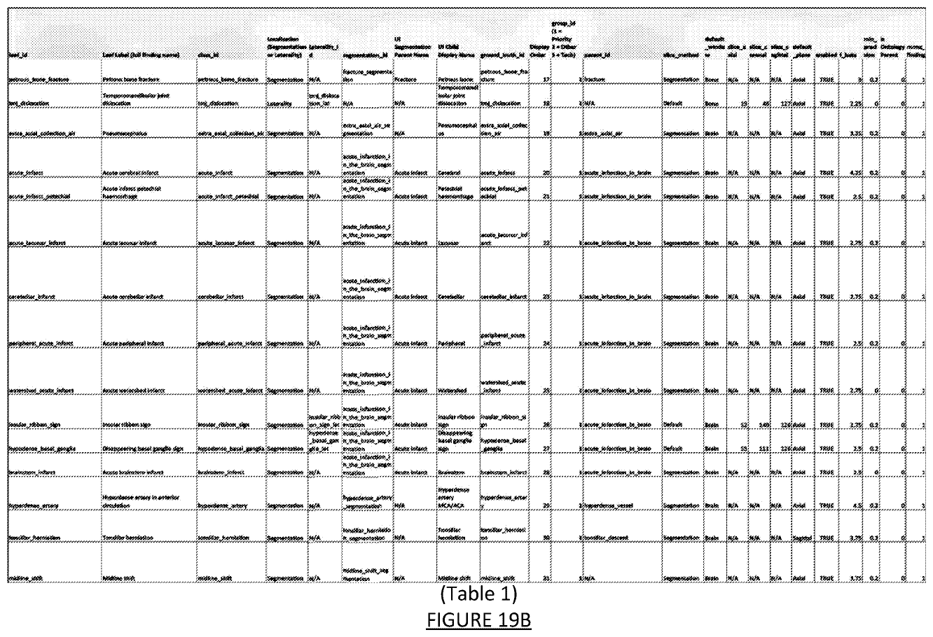

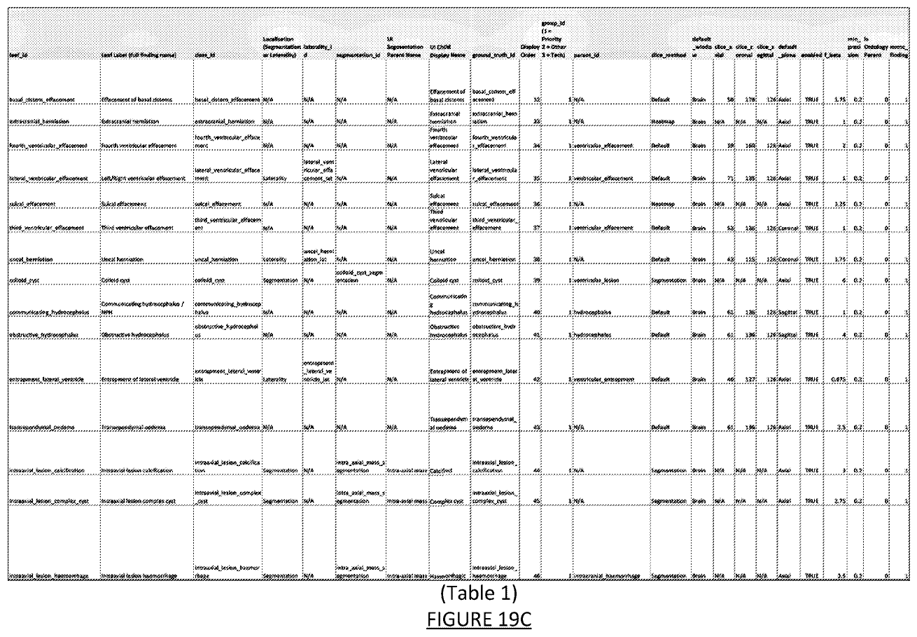

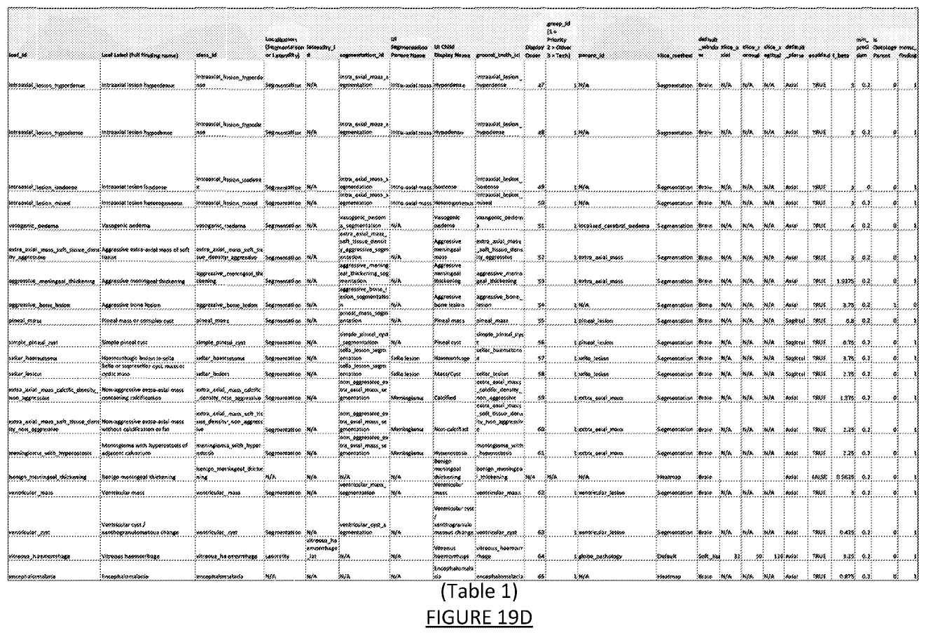

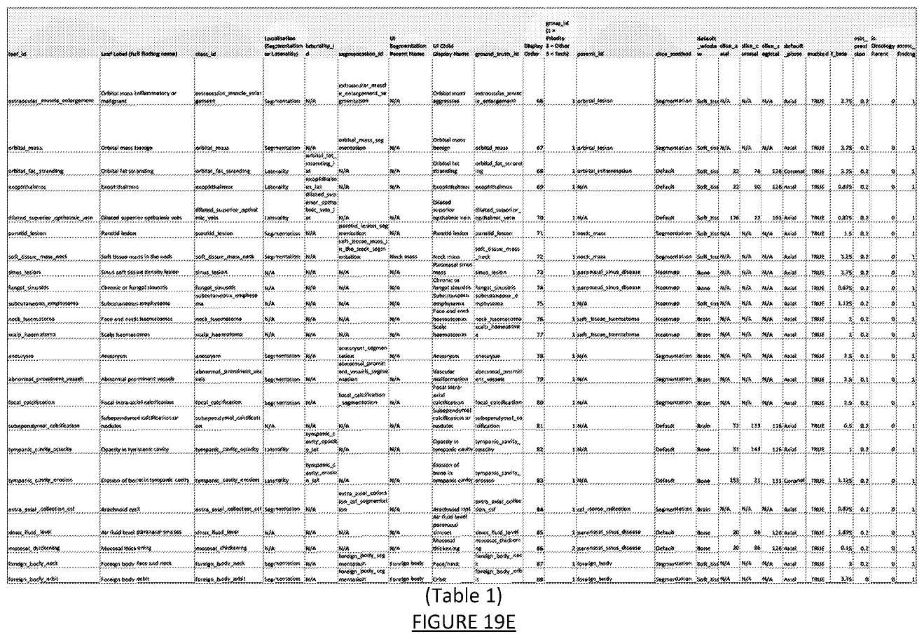

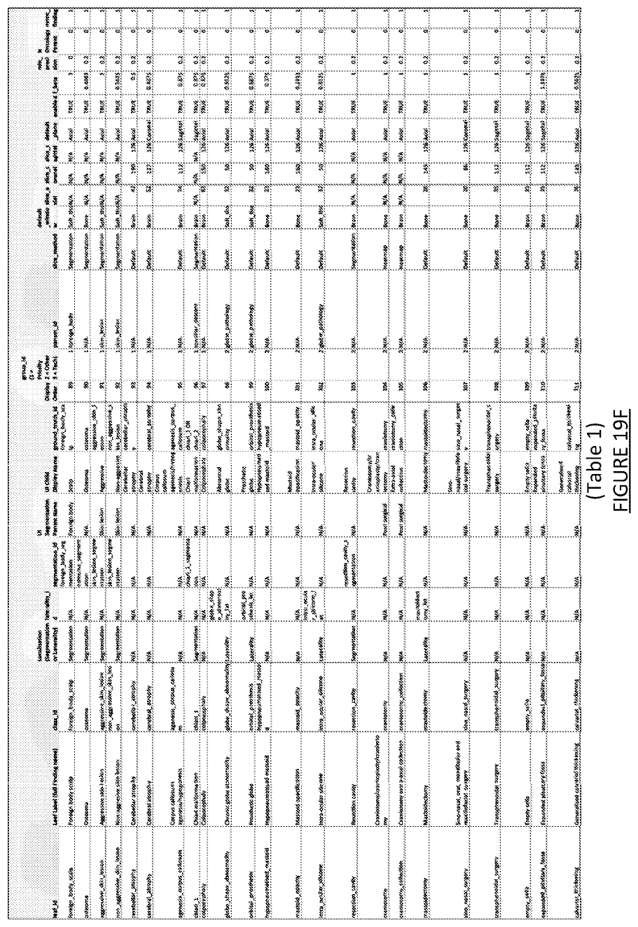

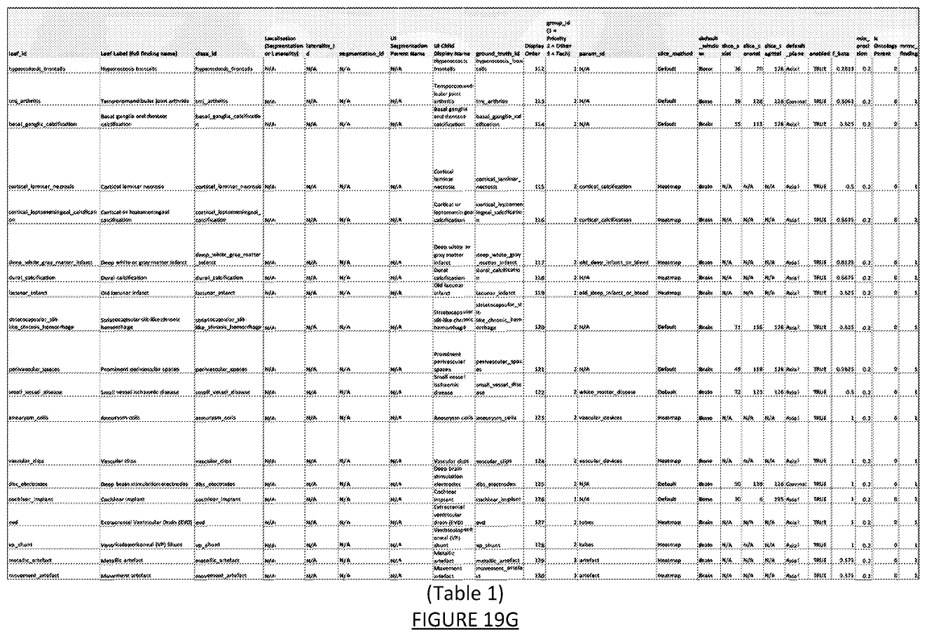

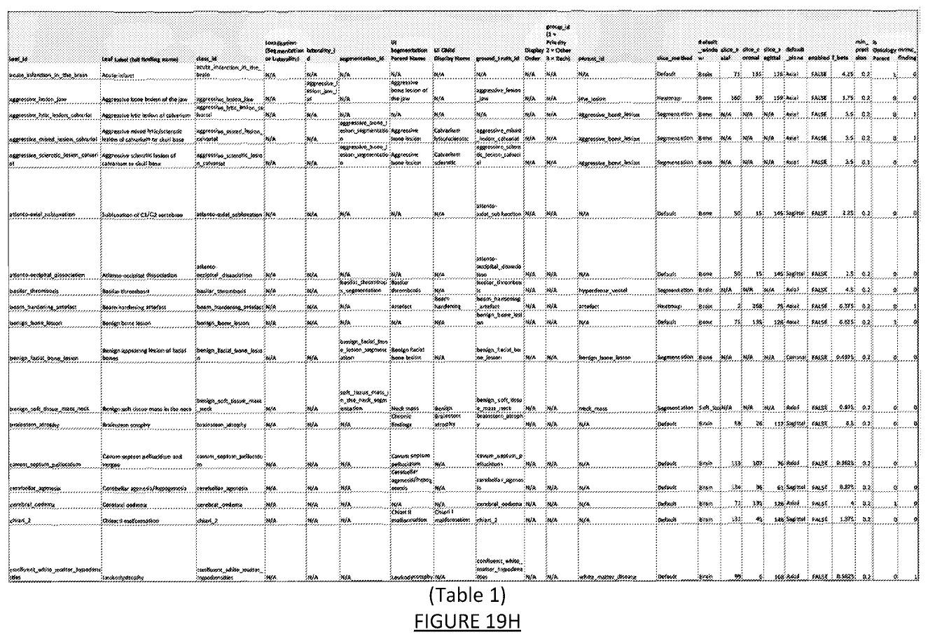

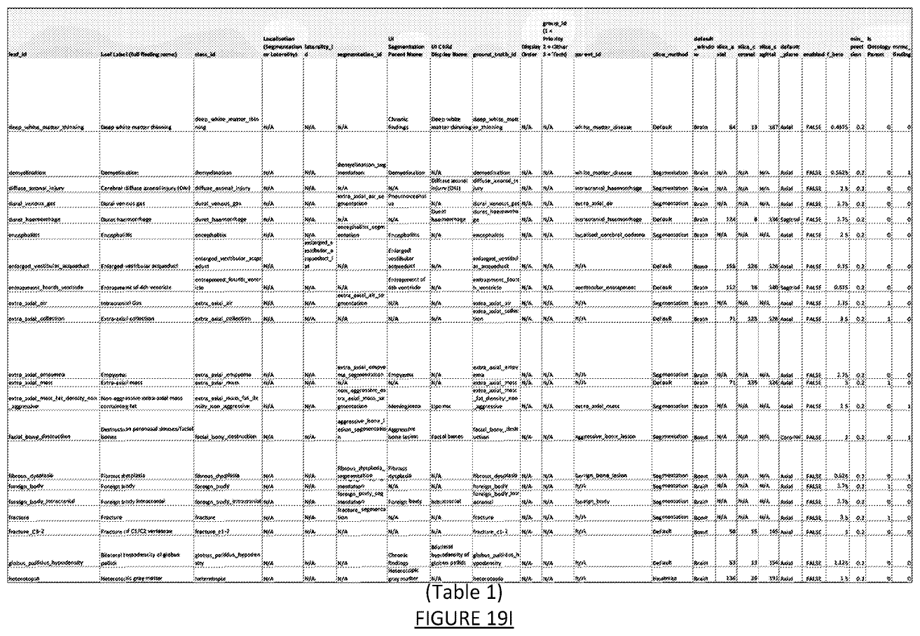

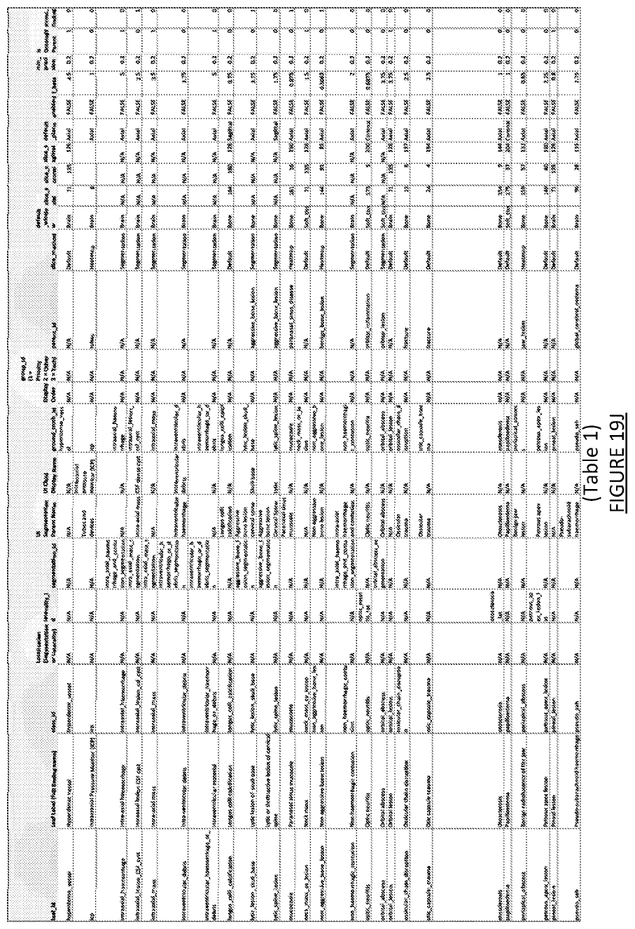

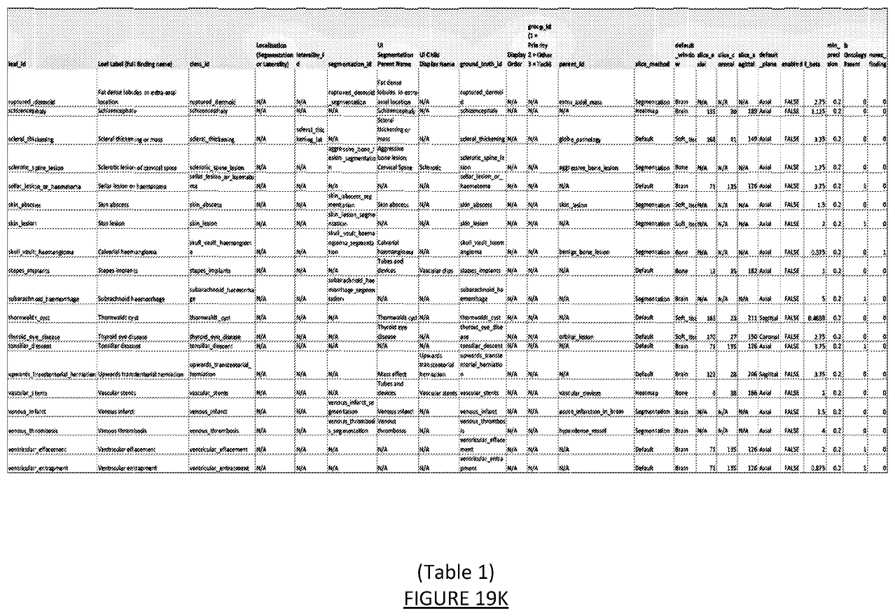

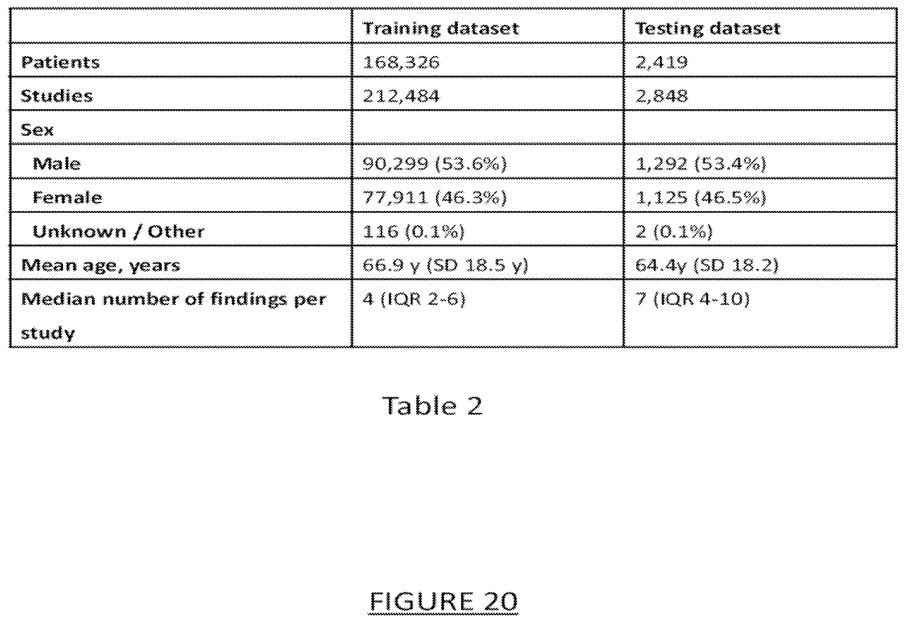

FIG. 18 illustrates the 3D functionality of the CNN model, visualising a single case with multiple intracranial findings by way of 3D segmentation masks. FIG. 18 illustrates a 3D visualisation of an infarcted area demonstrating the 3D functionality of the model.

The CNN model 200 can be configured for generating, using at least one of the 3D feature tensors by the CNN decoder 306: a decoder 3D tensor. The CNN model 200 can be configured for generating, using the decoder 3D tensor by a segmentation module: one or more 3D segmentation masks, each 3D segmentation mask representing a localization in 3D space of a respective one of the visual anomaly findings classified as being present by the segmentation module. As shown in FIG. 18, on the viewer component 701, the 3D segmentation mask can be overlaid onto the spatial 3D tensor (or a 3D spatial model) of the head of the subject, by way of 3D representation.

In the study, the prevalence of findings in the training set was the limiting factor in their inclusion in the model. Of 192 child findings that were originally labelled for training, only 144 were present in sufficient numbers to allow successful model training and verification. Further performance evaluation resulted in the inclusion of 120 findings in the final production model. Since an extensive real-world database was used to extract the cases for training, however, the lack of prevalence for a finding indicated that it would rarely be encountered by a reporting clinician in their daily practice. As is normally required by the clinician, a certain level of vigilance for these rare entities would still be required when using the CNN model 200 as an aid to detection of pathology and diagnosis.

The model was validated in a large MRMC study involving 32 radiologists labelling 2,848 NCCTB scans. Reader performance, when unaided by the model, varied enormously depending on the subtlety of the finding, ranging from an average AUC of 0.50 for “intraventricular debris” to an AUC of 0.97 for “DBS electrodes”. The average AUC for unaided readers across all findings was 0.68. The average AUC for the model was considerably better at 0.93. The average AUC across the parent findings was 0.73 and 0.90 for the unassisted radiologists and the model, respectively. When aided by the model, radiologists significantly improved their performance across 49% of findings. Model accuracy can be attributed to the large training set of 212,484 studies, each individually labelled for 192 findings by multiple radiologists. The radiologists were initially trained to conform to tight definitions of each label with regular updates throughout the labelling procedure to reinforce definitions.

Model benefits were most pronounced when aiding radiologists in the detection of subtle findings. The low unaided radiologist AUC of 0.57 for “watershed infarct” indicated a performance that was little better than random guessing for this finding, which may not be surprising as these infarcts were generally subtle. Ground truth labelling for acute infarcts was usually aided by diffusion weighted MRI scans or follow up CT scans. Diffusion weighting is the most accurate method for detecting acute infarcts as it detects signal related to microscopic changes in the tissue. CT does not have this ability and relies on macroscopic tissue changes to produce a change in density. However, as infarcts age, they become more visible, allowing for clearer detection on follow up CT studies. Model performance for “watershed infarct”, with an AUC of 0.91(0.87−0.94), indicated that although this finding proved difficult for radiologists to detect, subtle abnormalities were generally present on the CT scan that allowed detection by the model. The AUC for augmented readers was 0.68. The considerable improvement of the radiologists in detecting these infarcts when aided by the model suggests that the findings on these studies were visible to the human eye, even though often missed in the unaided arm of the study. A large increase in AUC from reader to model was also seen for the other labels signifying the presence of acute infarcts, including increases of AUC between 0.11 for “acute cerebral infarct” to 0.17 for “insular ribbon sign” which is one of the earliest signs of acute infarction on CT. Evidently, “insular ribbon sign” was difficult for radiologists to detect (AUC 0.55) due to the method of ground truthing, as any MRI diffusion abnormality in the insular cortex was labelled as positive for this finding, regardless of the appearance on CT. However, the model performed exceptionally well on this finding with an AUC of 0.95, again confirming that the model was capable of detecting abnormalities on the CTB studies that generally eluded radiologists. Further work is required to investigate the gap between augmented reader and model performance.

In radiology, there is often a trade-off between recall and precision. Radiologists can improve their recall rate by calling more subtle findings, but this results in false positives, reducing precision. Usually, the balance is struck with the level of precision being higher than the level of recall. This is due to the majority of errors in radiology being errors of visual perception rather than cognitive errors. Errors of visual perception are comprised of false negatives, reducing the recall rate of the radiologist. Visual search favours some parts of the image over others. Conversely, CNNs tend to treat all parts of an image with the same level of scrutiny and can alert the radiologist to findings they would otherwise miss, raising their level of recall without the disadvantage of significantly reducing precision, as radiologists are proficient at excluding false positives. Results suggested that increasing the F-beta of the model improved radiologist sensitivity without significantly compromising precision. NCCTB scans are usually part of the initial investigation of acute stroke. However, these studies yield a low sensitivity for detection of acute cerebral infarction, even for experienced eyes. Stroke management relies on knowledge of the site and size of the infarct, often relying on other modalities such as CT perfusion or MRI for these answers. The study demonstrated that subtle findings often present on the NCCTB study can be brought to the attention of the clinician using the CNN model 200.

Interestingly, many of the largest gains in performance by the radiologists when using the AI, were for clinically critical findings, including findings signifying mass effect (basal cistern effacement, tonsillar and uncal herniation, midline shift, sulcal and ventricular effacement), findings related to intracranial haemorrhage (aneurysmal and convexity subarachnoid haemorrhage), trauma (haemorrhagic contusions, acute subdural haematomas, petrous bone fractures and subcutaneous emphysema), obstructive hydrocephalus and transependymal oedema, intra-axial lesions (haemorrhagic, mixed, isodense and hypodense masses, colloid cyst and vasogenic oedema), aggressive extra-axial masses,), orbital cellulitis and acute infarction (insular ribbon sign, disappearing basal ganglia sign, hyperdense artery, acute watershed infarct and acute cerebral infarct).

The study demonstrated the successful deployment of an example of the CNN model 200 in a controlled setting to aid radiologists in the detection of a comprehensive list of abnormalities on non-contrast CT scans of the brain.

Chilamkurthy, S. et al., Deep learning algorithms for detection of critical findings in head CT scans: a retrospective study; Lancet 392, 2388-2396 (2018), herein incorporated by reference herein in its entirety, trained and validated a deep learning model that was capable of detecting four critical clinical findings, using a dataset consisting of 313,318 NCCTB scans.

While example embodiments have been described in conjunction with the exemplary embodiments described above, many equivalent modifications and variations will be apparent to those skilled in the art. Accordingly, the exemplary embodiments set forth above are considered to be illustrative and not limiting. Various changes to the described embodiments may be made without departing from the scope of the example embodiments.

For the avoidance of any doubt, any theoretical explanations provided herein are provided for the purposes of improving the understanding of a reader. The inventors do not wish to be bound by any of these theoretical explanations.

Any section headings used herein are for organisational purposes only and are not to be construed as limiting the subject matter described.

The units described as separate parts may or may not be physically separate, and parts displayed as units may or may not be physical units, may be located in one position, or may be distributed on a plurality of network units. Some or all of the units may be selected according to actual requirements to achieve the objectives of the solutions of the embodiments.

In addition, functional units in the example embodiments may be integrated into one processing unit, or each of the units may exist alone physically, or two or more units are integrated into one unit.

When the functions are implemented in the form of a software functional unit and sold or used as an independent product, the functions may be stored in a computer-readable storage medium. Based on such an understanding, the technical solutions of example embodiments may be implemented in a form of a software product. The software product is stored in a storage medium, and includes several instructions for instructing a computer device (which may be a personal computer, a server, or a network device) to perform all or some of the steps of the methods and access control methods described in the example embodiments. The foregoing storage medium includes any medium that can store program code, such as a Universal Serial Bus (USB) flash drive, a removable hard disk, a read-only memory (Read-Only Memory, ROM), a random access memory (Random Access Memory, RAM), a magnetic disk, or an optical disc.

In the described methods or block diagrams, the boxes may represent events, steps, functions, processes, modules, messages, and/or state-based operations, etc. While some of the example embodiments have been described as occurring in a particular order, some of the steps or processes may be performed in a different order provided that the result of the changed order of any given step will not prevent or impair the occurrence of subsequent steps. Furthermore, some of the messages or steps described may be removed or combined in other embodiments, and some of the messages or steps described herein may be separated into a number of sub-messages or sub-steps in other embodiments. Even further, some or all of the steps may be repeated, as necessary. Elements described as methods or steps similarly apply to systems or subcomponents, and vice-versa. Reference to such words as “sending” or “receiving” could be interchanged depending on the perspective of the particular device.

Any discussion of documents, acts, materials, devices, articles or the like which has been included in the present specification is not to be taken as an admission that any or all of these matters form part of the prior art base or were common general knowledge.

Throughout this specification the words “tensor” and “vector” may be used interchangeably unless otherwise indicated. Example illustrative tensor dimensions are shown in parentheses or square brackets in the Figures, and are not intended to be limiting. Reference to dimension such as a 2D or 3D can include more than those number of dimensions as applicable. A plurality of 3D tensors can be arranged within a 4D tensor (or higher dimension). A plurality of 2D tensors can be arranged within a 3D tensor (or higher dimension).

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The described embodiments are considered to be illustrative and not restrictive. Example embodiments described as methods would similarly apply to systems or devices, and vice-versa.

The various example embodiments are merely examples and are in no way meant to limit the scope of the example embodiments. Variations of the innovations described herein will be apparent to persons of ordinary skill in the art, such variations being within the intended scope. In particular, features from one or more of the example embodiments may be selected to create alternative embodiments comprises of a sub-combination of features which may not be explicitly described. In addition, features from one or more of the described example embodiments may be selected and combined to create alternative example embodiments comprised of a combination of features which may not be explicitly described. Features suitable for such combinations and sub-combinations would be readily apparent to persons skilled in the art upon. The subject matter described herein intends to cover all suitable changes in technology.

1. A method for visual detection, the method being performed by at least one processor and comprising:
receiving a series of anatomical images obtained from a computed tomography (CT) scan of a head of a subject;
generating, using the series of anatomical images by a preprocessing layer: a spatial 3D tensor which represents a 3D spatial model of the head of the subject;
generating, using the spatial 3D tensor by a convolutional neural network (CNN) model: at least one 3D feature tensor; and
classifying, using at least one of the 3D feature tensors by the CNN model: each of a plurality of possible visual anomaly findings as being present versus absent, the plurality of possible visual anomaly findings having a hierarchal relationship based on a hierarchical ontology tree.

2. The method of claim 1, further comprising modifying, using the hierarchical ontology tree, when a first possible visual anomaly finding at a first hierarchal level of the hierarchical ontology tree is classified by the CNN model to be present and a second possible visual anomaly finding at a second hierarchal level of the hierarchical ontology tree that is higher than the first hierarchal level is classified by the CNN model to be absent, the classifying of the second possible visual anomaly finding to being present.

3. The method of claim 2, further comprising updating a training of the CNN model using the series of anatomical images labelled with the first possible visual anomaly finding as being present and the second possible visual anomaly finding as being present.

4. The method of claim 2, wherein the CNN model is trained through a labelling tool for a plurality of sample CT images that allows at least one expert to select labels presented in a hierarchical menu which displays at least some of the possible visual anomaly findings in the hierarchal relationship from the hierarchical ontology tree, in which labelling of a first possible visual anomaly label at the first hierarchal level of the hierarchical ontology tree as being present automatically labels a second possible visual anomaly label at the second hierarchal level of the hierarchical ontology tree as being present.

5. The method of claim 2, wherein the first hierarchal level of the hierarchical ontology tree comprises terminal leaves and the second hierarchal level of the hierarchical ontology tree comprises internal nodes, wherein each internal node uniquely branches to one or more terminal leaves.

6. The method of claim 1, further comprising generating for display the plurality of possible visual anomaly findings classified as being present by the CNN model in the hierarchal relationship defined by the hierarchical ontology tree.

7. The method of claim 1, wherein the hierarchical ontology tree comprises Table 1.

8. The method of claim 1, wherein the hierarchical ontology tree lists the possible visual anomaly findings.

9. The method of claim 1, wherein the generating of the at least one of the 3D feature tensors is performed by a CNN encoder.

10. The method of claim 9, further comprising:
generating, using at least one of the 3D feature tensors by a CNN decoder of the CNN model: a decoder 3D tensor; and
generating, using the decoder 3D tensor by a segmentation module: one or more 3D segmentation masks, each 3D segmentation mask representing a localization in 3D space of a respective one of the visual anomaly findings classified as being present by the segmentation module.

11. The method of claim 10, further comprising:
generating, using the 3D segmentation mask: segmentation maps in at least one anatomical plane of each respective visual anomaly finding classified as being present.

12. The method of claim 11, wherein the segmentation map is generated for each of the respective visual anomaly finding with segmentation as indicated in Table 1 and classified as being present.

13. The method of claim 11, wherein each of the segmentation maps is a binary mask.

14. The method of claim 11, wherein the at least one anatomical plane is: sagittal, coronal, or transverse.

15. The method of claim 11, further comprising:
generating, using at least one of the 3D feature tensors and a vision transformer: an attention weight 3D tensor; and
generating, using the attention weight 3D tensor by a key slice generator: a default anatomical slice from the CT scan to be displayed as a default slice or key slice for at least one of the visual anomaly finding classified as being present;
generating for display at least one of the segmentation maps overlaid on the default anatomical slice from the CT scan in at least one of the anatomical planes.

16. The method of claim 10, further comprising:
generating, using at least one of the 3D feature tensors by a vision transformer: a vision tensor; and
wherein the generating the decoder tensor by the CNN decoder further uses the vision tensor.

17. The method of claim 9, further comprising:
generating, using at least one of the 3D feature tensors by a vision transformer: a flattened tensor; and
generating, using the flattened tensor by a laterality classification head, a left-right laterality of at least one of the possible visual anomaly findings classified as being present.

18. The method of claim 17, wherein the left-right laterality is generated for each of the possible visual anomaly findings with laterality as indicated in Table 1 and classified as being present.

19. The method of claim 9, further comprising:
generating, using at least one of the 3D feature tensors by a vision transformer: a flattened tensor; and
wherein the classifying is performed by a classification module further using the flattened tensor.

20. The method of claim 9, further comprising:
generating, using at least one of the 3D feature tensors and a vision transformer: attention weights; and
generating, using the attention weights by a key slice generator: an anatomical slice from the CT scan to be displayed as a default view for at least one of the visual anomaly finding classified as being present.

21.-48. (canceled)
